SEQUENCE LISTING (24 sequences)

SEQ ID NO: 1 - DNA, 12 bp, Artificial Sequence
  Synthetic sequence
aaggccttaagg

SEQ ID NO: 2 - DNA, 17 bp, Artificial Sequence
  Synthetic sequence
  misc_feature (1)..(1): 17-O-(4,4′-Dimethoxytrityl)-hexaethyleneglycol, 1-[(2-cyanoethyl)-(N,N-diisopropyl)]-phosphoramidite (HEG)
  misc_feature (11)..(12): 17-O-(4,4′-Dimethoxytrityl)-hexaethyleneglycol, 1-[(2-cyanoethyl)-(N,N-diisopropyl)]-phosphoramidite (HEG)
aaaaaaggcgcgcgccu

SEQ ID NO: 3 - DNA, 16 bp, Artificial Sequence
  Synthetic sequence
  misc_feature (8)..(9): 17-O-(4,4′-Dimethoxytrityl)-hexaethyleneglycol, 1-[(2-cyanoethyl)-(N,N-diisopropyl)]-phosphoramidite (HEG)
gaagagctagctcttc

SEQ ID NO: 4 - DNA, 14 bp, Artificial Sequence
  Synthetic sequence
  misc_feature (7)..(8): 17-O-(4,4′-Dimethoxytrityl)-hexaethyleneglycol, 1-[(2-cyanoethyl)-(N,N-diisopropyl)]-phosphoramidite (HEG)
tgtacatatgtaca

SEQ ID NO: 5 - DNA, 20 bp, Artificial Sequence
  Synthetic sequence
gtggtatcaacgcagagtac

SEQ ID NO: 6 - DNA, 20 bp, Artificial Sequence
  Synthetic sequence
gtccgagcgtaggttatccg

SEQ ID NO: 7 - DNA, 104 bp, Artificial Sequence
  Synthetic sequence
  misc_feature (2)..(3): 17-O-(4,4′-Dimethoxytrityl)-hexaethyleneglycol, 1-[(2-cyanoethyl)-(N,N-diisopropyl)]-phosphoramidite (HEG)
  misc_feature (2)..(3): 3-(4,4′-Dimethoxytrityl)-1-(2-nitrophenyl)- propane-1,3-diol-[2-cyanoethyl-(N,N-diisopropyl)]-phosphoramidite (PC)
  misc_feature (2)..(3): 17-O-(4,4′-Dimethoxytrityl)-hexaethyleneglycol, 1-[(2-cyanoethyl)-(N,N-diisopropyl)]-phosphoramidite (HEG)
  misc_feature (35)..(58): dimer nucleotide added via split and pool synthesis (J)
  misc_feature (59)..(74): degenerate dimer nucleotide (N)
tttttttttaagcagtggtatcaacgcagagtacnnnnnnnnnnnnnnnnnnnnnnnnnnnnnnnnnnnnnnnntttttttttttttttttttttttttttttt

SEQ ID NO: 8 - DNA, 23 bp, Artificial Sequence
  Synthetic sequence
aagcagtggtatcaacgcagagt

SEQ ID NO: 9 - DNA, 37 bp, Artificial Sequence
  Synthetic sequence
gcctgtccgcggaagcagtggtatcaacgcagagtac

SEQ ID NO: 10 - DNA, 40 bp, Artificial Sequence
  Synthetic sequence
  misc_feature (25)..(40): n is a, c, g, or t
ccccccccccccccccccccccccnnnnnnnnnnnnnnnn

SEQ ID NO: 11 - DNA, 30 bp, Artificial Sequence
  Synthetic sequence
  misc_feature (28)..(30): ribonucleic acid Guanine (rG)
aagcagtggtatcaacgcagagtgaatnnn

SEQ ID NO: 12 - DNA, 66 bp, Artificial Sequence
  Synthetic sequence
  misc_feature (65)..(66): phosphorothioated DNA nucleotides
aatgatacggcgaccaccgagatctacacgcctgtccgcggaagcagtggtatcaacgcagagtac

SEQ ID NO: 13 - DNA, 139 bp, Artificial Sequence
  Synthetic sequence
  misc_feature (36)..(59): dimer nucleotide added via split and pool synthesis (J)
  misc_feature (60)..(71): degenerate dimer nucleotide
  misc_feature (127)..(128): 17-O-(4,4′-Dimethoxytrityl)-hexaethyleneglycol, 1-[(2-cyanoethyl)-(N,N-diisopropyl)]-phosphoramidite (HEG)
  misc_feature (138)..(139): 17-O-(4,4′-Dimethoxytrityl)-hexaethyleneglycol, 1-[(2-cyanoethyl)-(N,N-diisopropyl)]-phosphoramidite (HEG)
  misc_feature (138)..(139): 3-(4,4′-Dimethoxytrityl)-1-(2-nitrophenyl)- propane-1,3-diol-[2-cyanoethyl-(N,N-diisopropyl)]-phosphoramidite (PCLinker)
tcggaccgttcgtcggtggtatcaacgcagagtacnnnnnnnnnnnnnnnnnnnnnnnnnnnnnnnnnnnngtccgagcgtaggttatccgttttttttttttttttttttttttttttttuccgcgcgcggaaaaaat

SEQ ID NO: 14 - DNA, 24 bp, Artificial Sequence
  Synthetic sequence
tcgtcggcagcgtcagatgtgtat

SEQ ID NO: 15 - DNA, 20 bp, Artificial Sequence
  Synthetic sequence
cggataacctacgctcggac

SEQ ID NO: 16 - DNA, 86 bp, Artificial Sequence
  Synthetic sequence
caagcagaagacggcatacgagattcgccttagtctcgtgggctcggagatgtgtataagagacagcggataacctacgctcggac

SEQ ID NO: 17 - DNA, 50 bp, Artificial Sequence
  Synthetic sequence
aatgatacggcgaccaccgagatctacactcgtcggcagcgtcagatgtg

SEQ ID NO: 18 - DNA, 14 bp, Artificial Sequence
  Synthetic sequence
  misc_feature (1)..(1): 5′-phosphate (P)
  misc_feature (14)..(14): Inverted A (InvA)
gacgctgccgacgn

SEQ ID NO: 19 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 20 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 21 - protein, 75 aa, Severe acute respiratory syndrome coronavirus 2
MYSFVSEETGTLIVNSVLLFLAFVVFLLVTLAILTALRLCAYCCNIVNVSLVKPSFYVYSRVKNLNSSRVPDLLV

SEQ ID NO: 22 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 23 - DNA, 40 bp, Artificial Sequence
  Synthetic sequence
aattaaggttccggttggccaaggccggttaaccggggcc

SEQ ID NO: 24 - DNA, 17 bp, Artificial Sequence
  Synthetic sequence
aaaaaaaaaaaaaaaaa

(19) United States
(12) Patent Application Publication
(10) Pub. No.: US 2024/0102091 A1
(43) Pub. Date: Mar. 28, 2024

(54) OLIGONUCLEOTIDES

(71) Applicant: OXFORD UNIVERSITY INNOVATION LIMITED, Oxford (GB)

(72) Inventors: Martin PHILPOTT, Oxford (GB); Adam CRIBBS, Oxford (GB); Udo OPPERMANN, Oxford (GB); Tom BROWN, Jr., Oxford (GB); Tom BROWN, Sr., Oxford (GB); Jonathan Francis WATSON, Oxford (GB)

(21) Appl. No.: 18/039,846

(22) PCT Filed: Dec. 2, 2021
(86) PCT No.: PCT/GB2021/053152

§ 371 Date: Jun. 1, 2023

(30) Foreign Application Priority Data
Dec. 2, 2020 (GB) 2019035.1

(51) Int. Cl. C12Q 1/6874; C12Q 1/6876

(52) U.S. Cl. CPC C12Q 1/6874; C12Q 1/6876; C12Q 1/6806

(57) ABSTRACT

The invention relates to methods of adding identifier sequences to polynucleotides of an array. The identifier sequences comprise a plurality of nucleotide blocks. Also provided are arrays of polynucleotides having identifier sequences, microparticles comprising said arrays, a plurality of 5 said microparticles, surfaces comprising said arrays, kits and methods for generating libraries using the array, methods for determining the accuracy of sequencing or amplification an array, and methods of analysing said libraries.

### FIELD OF THE INVENTION

The disclosure relates to polynucleotide identifier sequences, such as barcode sequences (BC) and unique molecular identifier sequences (UMIs), methods of generating polynucleotides comprising such identifier sequences, arrays of polynucleotides comprising identifier sequences, methods of generating libraries of polynucleotides or analytes including or using the identifier sequences, and uses thereof.

### BACKGROUND TO THE INVENTION

Single-cell RNA sequencing (scRNA-seq) is a widely adopted method for profiling the transcriptome in both health and disease. Current scRNA-seq methods can be broadly categorised as well-based or droplet-based. Well-based methods, such as SMART-seq, sort single cells into individual wells of a multi-well plate, which act as a discrete reaction vessel for subsequent library production, followed by short-read sequencing. SMART-seq has the advantage of coverage of full-length transcripts, although inferring individual transcripts from short-read sequencing remains challenging and the method is limited to processing hundreds of cells at a very high cost per cell. Droplet-based methods, such as Drop-Seq or 10× Genomics Chromium, co-capture cells and oligonucleotide-barcoded RNA-capture microbeads in droplets within an oil emulsion, where each droplet becomes a discrete reaction vessel for associating a different barcode with each cell's RNA, followed by pooled library production and short-read sequencing. Barcoded RNA-capture microbeads for Drop-seq are manufactured using a manual split and pool process to create a unique-to-each-bead barcode region within the capture-oligonucleotide sequence. These droplet-based methods are capable of reporting on many thousands of cells at a dramatically reduced cost per cell, but are only capable of reporting on the 5′ or 3′ ends of transcripts.

Droplet based single-cell sequencing techniques have provided unprecedented insight into cell-to-cell heterogeneity within tissues. However, these approaches only allow for the measurement of the extremity of a transcript following short-read sequencing. Therefore, splicing information and the ability to measure sequence diversity is lost for the majority of the transcript. The application of long-read nanopore sequencing to droplet-based methods is challenging because of the low basecalling accuracy. Several approaches that use short read sequencing to error correct the barcode and UMI sequences have been developed. However, these techniques are limited by the requirement to sequence a library using both short-read and long-read sequencing technologies.

Long-read sequencing platforms, such as Oxford Nanopore, have the potential to deliver the throughput and economy of droplet-based short-read sequencing and provide true end-to-end sequencing of transcripts, allowing examination of RNA splicing, single nucleotide polymorphisms, structural variation, imprinting and chimeric transcripts at the single-cell level. Furthermore, long-read sequencing also allows users the ability to more accurately perform mutational, translocation, copy number variation and allele frequency at the single-cell level. However, the drawback of Nanopore sequencing is its high error rate compared to Illumina short-read sequencing (5-15% vs <1%) [1]. For many applications, the advantages of long reads outweigh the low-read accuracy or can be overcome with consensus sequences from homogenous samples. However, in scRNA-seq, the fidelity of the barcode and UMI region is critical, which has hampered the adoption of Nanopore sequencing at the single-cell level.

Several groups have reported using parallel short-read Illumina sequencing to error correct long-read Nanopore single-cell sequencing [2; 3; 4]. While this approach was able to increase assignment rates from just ˜6% to ˜67%, the requirement to independently construct and sequence two libraries raises the time and cost of single-cell sequencing. Moreover, accurate UMI assignment is incredibly challenging with this approach because of the random nature of the UMI generation and low basecalling accuracy of nanopore. Volden et al [5] used a Rolling Circle Amplification to Concatemeric Consensus (R2C2) method to error correct Nanopore sequencing. Although this method achieved 96% sequencing accuracy, this still only translated to 72% of barcodes demultiplexing correctly, with 45% of UMIs not matching to parallel Illumina sequencing. Furthermore, the increased read length needed to support this error correction approach is prone to increased error rates for longer reads in the late stages of a sequencing run [6].

### SUMMARY OF THE INVENTION

To help fully realise the potential of long-read single-cell sequencing, the inventors have developed a method whereby the critical identifier sequences, such as the barcode and/or UMI regions, can be better identified and grouped despite errors introduced by amplification and/or sequencing. The method involves using an identifier sequence that is built up using one or more pools of nucleotide blocks having a mixture of pre-selected sequences, wherein each nucleotide block sequence in a pool differs from each other nucleotide block sequence in the pool by at least two nucleotide substitutions. This allows single errors in the sequence to be detected and accounted for, and the overall error rate to be determined. This allows a greater number of sequenced polynucleotides to be correctly assigned using the identifier sequence. The method also allows the correction of long-read single cell sequencing, without the need for parallel short-read sequencing.

Accordingly, in a first aspect, the invention provides
1. A method adding of at least one identifier sequence to each of an array of polynucleotides during synthesis, wherein the identifier sequence comprises a plurality of nucleotide blocks, wherein the method comprises adding each nucleotide block of the identifier sequence by elongating the polynucleotides using a pool of pre-synthesised nucleotide blocks for incorporation into the polynucleotides, wherein the pool of pre-synthesised nucleotide blocks used to add each nucleotide block of the identifier sequence comprises a mixture of different nucleotide block sequences, wherein each nucleotide block sequence in the pool used to add each nucleotide block of the identifier sequence differs from each other nucleotide block sequence in the pool by at least two nucleotide substitutions, and wherein the method adds a different identifier sequence to each of at least 100 different polynucleotides of the array.

Using pre-synthesised blocks, rather than additional rounds of polynucleotide synthesis to build the nucleotide blocks, reduces labour and costs and increases yield, particularly when using split-and-pool polynucleotide synthesis to generate the identifier sequence. Alternatively, if the sequence is randomly generated, such as a UMI sequence generated using degenerate polynucleotide synthesis, using pre-synthesised blocks makes it possible to identify and correct errors.

In further aspect and embodiments the invention provides for following
2. The method of item 1, wherein the pre-synthesised nucleotide blocks comprise nucleotide phosphoramidites.
3. The method according to item 1 or item 2, wherein the at least one identifier sequence is added to the polynucleotides using degenerate polynucleotide synthesis using the mixed pool of pre-synthesised nucleotide blocks.
4. The method according to any one of the preceding items, wherein the at least one identifier sequence is added using split-and-pool polynucleotide synthesis comprising
   (i) splitting the array of polynucleotides or the sub-arrays into groups;
   (ii) combining each group with a different sub-pool of the pre-synthesised nucleotide blocks, wherein the pre-synthesised nucleotide blocks of each sub-pool have different nucleotide block sequences;
   (iii) polynucleotide synthesis to add one pre-synthesised nucleotide block from the sub-pools to the polynucleotides of each respective group;
   (iv) isolating the polynucleotides of the array from the sub-pools of pre-synthesised nucleotide blocks;
   (v) optionally mixing the polynucleotides from the groups together; and
   (vi) repeating steps (i) to (v), allocating different combinations of the polynucleotides or sub-arrays to different groups compared to the previous round.
5. An array of polynucleotides, wherein each polynucleotide of the array comprises an identifier sequence, wherein the identifier sequence of each polynucleotide consists of a consecutive series of at least three nucleotide blocks, wherein each nucleotide block of the identifier sequences is selected from a pool of up to 36 nucleotide block sequences, wherein each nucleotide block sequence of the pool differs from each other nucleotide block sequence of the pool by at least two nucleotide substitutions, and wherein the array comprises at least 100 polynucleotides each having a different identifier sequence.
6. The method according to any one of items 1 to 4, or the array of polynucleotides according to item 5, wherein each nucleotide block consists of two or more of the same nucleotide.
7. The method or array of polynucleotides according to any one of items 1 to 6, wherein at least one identifier sequence of or added to each polynucleotide of the array is a unique molecular identifier sequence (UMI).
8. The method or array of polynucleotides according to item 7, wherein
   (a) the UMI sequence of or added to each polynucleotide is different to the UMI sequence of or added to essentially each other polynucleotide in the array; and/or
   (b) the polynucleotide array is divided into a plurality of sub-arrays, wherein the UMI sequence of or added each polynucleotide in each sub-array is different from the UMI sequence of or added to essentially each other polynucleotide of the same sub-array; optionally wherein each polynucleotide further comprises, or the method further comprises adding to each polynucleotide, a barcode sequence (BC), wherein the BC sequence of each polynucleotide is the same as the BC of essentially each other polynucleotide of the same sub-array, but different from the BC sequence of the polynucleotides of essentially every different sub-array, and wherein the BC sequence and the UMI sequence are in or added in either order; further optionally wherein both the UMI sequences and the BC sequences are or are added by a method of any one of items 1 to 4.
9. The method or array of polynucleotides according to any one of items 1 to 8, wherein the polynucleotide array is divided into a plurality of sub-arrays, wherein at least one identifier sequence of or added to each polynucleotide is a barcode sequence (BC), wherein the BC sequence of or added to the polynucleotides of each sub-array is the same as the BC sequence of or added to essentially each other polynucleotide of the same sub-array, but different from the BC sequence of or added to the polynucleotides of essentially every different sub-array; optionally wherein each polynucleotide further comprises, or the method further comprises adding to each polynucleotide, a UMI sequence, further optionally wherein the UMI sequence of or added to each polynucleotide in each sub-array is different from the UMI sequence of essentially each other polynucleotide of or added to the same sub-array, and wherein the BC sequence and the UMI sequence are or are added in either order.
10. The method or array of polynucleotides according to any one of items 1 to 9, wherein the array or each sub-array of polynucleotides is bound to a single micro-bead or associated with a single well or single pre-determined discrete position on a surface.
11. The method according to item 8 or item 9, wherein the method comprises adding the UMI sequence by degenerate polynucleotide synthesis and adding the BC sequence by split-and-pool polynucleotide synthesis.
12. The method or array of polynucleotides according to any one of items 1 to 11, wherein each polynucleotide is up to 1000 nucleotides in length.
13. The method or array of polynucleotides according to any one of items 1 to 12, wherein each polynucleotide is single-stranded.
14. The method or array of polynucleotides according to any one of items 1 to 13, wherein each polynucleotide further comprises:
   (a) an analyte capture region; and/or
   (b) a PCR handle sequence.
15. The method or array of item 14, wherein the polynucleotides comprise, in a 5′ to 3′ direction:
   (a) a PCR handle sequence;
   (b) a unique molecular identifier sequence (UMI), and/or a barcode sequence (BC), wherein the UMI is 5′ or 3′ to the BC; and
   (c) a 3′ end analyte capture region, optionally a polythymidine.
16. The method or array of item 14, wherein each polynucleotide comprises, in a 3′ to 5′ direction:
   (a) optionally a 3′ hydroxyl group, or a linker that is cleavable to provide a free 3′ hydroxyl group on the polynucleotide after cleavage;
   (b) a 3′ end analyte capture region, optionally wherein the 3′ end analyte capture region comprises:
      a. a polythymidine sequence;
      b. an aptamer;
      c. a sequence of at least 10 nucleotides for hybridising to a target polynucleotide analyte;
      d. a biotinylated nucleotide sequence; or
      e. an ATAC-med sequence.
   (c) optionally a first polymerase chain reaction (PCR) handle sequence;
   (d) a unique molecular identifier sequence (UMI), and/or a barcode sequence (BC), wherein the UMI is 5′ or 3′ to the BC;
   (e) optionally a (second) PCR handle sequence; and
   (f) optionally a 5′ end analyte capture region, optionally wherein the 5′ analyte capture region comprises:
      a. a polythymidine sequence;
      b. an aptamer;
      c. a sequence of at least 10 nucleotides for hybridising to a target polynucleotide analyte;
      d. a biotinylated nucleotide sequence; or
      e. an ATAC-med sequence.
17. The method or array of any one of items 1 to 16, wherein the identifier sequence is up to 14 nucleotide blocks in length.
18. A micro-particle comprising a micro-bead and an array of polynucleotides according to any one of items 5 to 10 and 12 to 17, wherein each polynucleotide is bound to the micro-particle.
19. The micro-particle of item 18, wherein each polynucleotide of the array has both a BC sequence and an UMI sequence, in either orientation, and wherein the BC sequence of essentially each polynucleotide of the array is the same, and optionally wherein the UMI sequence of essentially each polynucleotide of the array is different.
20. A plurality of micro-particles according to item 18 or item 19, wherein each polynucleotide has a BC sequence, wherein the BC sequence of each polynucleotide of each micro-particle is the same as the BC sequence of essentially each other polynucleotide of the same micro-bead, and different from the BC sequence of the polynucleotides of each other micro-particle.
21. A surface comprising a plurality of wells or discrete pre-determined positions, wherein each well or discrete pre-determined position is associated with an array of polynucleotides according to any one of items 5 to 10 and 12 to 17.
22. The surface according to item 21, wherein each polynucleotide has a BC sequence, wherein the BC sequence of each polynucleotide associated with each well or discrete pre-determined position is the same as the BC sequence of essentially each other polynucleotide of the same well or discrete pre-determined position, and different from the BC sequence of the polynucleotides associated with each other each well or discrete pre-determined position of the surface.
23. A kit for generating one or more libraries from one or more groups of analytes, the kit comprising an array of polynucleotides according to any one of items 5 to 10 and 11 to 17, a micro-particle according to item 18 or item 19, a plurality of micro-particles according to item 20, or a surface according item 21 or item 22.
24. A method of producing a library of polynucleotides, wherein the polynucleotides are amplified from the polynucleotides of a sample and/or tag non-polynucleotide analytes of a sample, and wherein the polynucleotides of the library include a barcode sequence (BC) and/or a unique molecular identifier sequence (UMI), the method comprising
   (a) capturing analytes in the sample on an array of polynucleotides synthesised according to any one of items 1 to 4 and 6 to 17, an array of polynucleotides according to any one of items 5 to 10 and 12 to 17, a micro-particle according to item 18 or item 19, a plurality of micro-particles according to item 20, or a surface according item 21 or item 22;
   (b) generating copies of the array of polynucleotides, including any sample polynucleotides captured by the array polynucleotides and the BC and/or UMI sequence(s);
   (c) amplifying the number of copies of each polynucleotide to produce a library of polynucleotides amplified from or tagging analytes in the sample and including the BC and/or UMI sequence.
25. The method of item 24, wherein the sample is a sample of cells, cell nuclei or cellular vesicles, a single cell, a single cell nucleus, a single vesicle, a tissue sample or tissue section, or a biological fluid sample, optionally a blood, blood fraction, serum, plasma, saliva or urine sample.
26. A library of polynucleotides produced by the method of item 24 or item 25.
27. A method of determining the accuracy of a method of amplifying and/or sequencing an array of polynucleotides of un-known sequence, the method comprising
   (a) including an identifier sequence in each polynucleotide of the array, wherein the identifier sequence comprises at least three nucleotide blocks at known block positions, wherein each nucleotide block of the identifier sequence comprises one of a pre-defined pool of nucleotide block sequences, wherein the pool of nucleotide block sequences at each block position differs from each other nucleotide block sequence in the pool by at least two nucleotide substitutions;
   (b) obtaining sequencing data for each polynucleotide or amplified polynucleotide, including the identifier sequence;
   (c) determining the percentage of the identifier sequences of the sequenced polynucleotides that are correctly sequenced as consisting only of one of the pre-defined nucleotide block sequences at each nucleotide bock position; and
   (d) using the percentage determined in step (c) to determine the accuracy of the method of amplification and/or sequencing and/or of the obtained polynucleotide sequences.
28. The method of item 27, further comprising using the percentage determined in step (c) to error correct polynucleotides sequences obtained in step (b) that are not correctly sequenced as consisting only of one of the pre-defined nucleotide block sequences at each nucleotide bock position.
29. The method of item 27 or item 28, wherein the array of polynucleotides comprises a plurality of sub-arrays, wherein the polynucleotides of each sub-array have a different identifier sequence.
30. The method of any one of items 27 to 29, further comprising allocating sequenced polynucleotides that meet the requirements of item 27(c) into groups, wherein the allocated sequenced polynucleotides within each group have the same sequenced identifier sequence, or the reverse complement thereof.
31. The method of 30, further comprising
   (a) using the percentage determined in item 27(c) to determine a cut-off for discarding polynucleotide sequences comprising more than the determined cut-off number of nucleotide blocks in the sequenced identifier sequence that have not been correctly sequenced as having one of the pre-selected sequences; and
   (b) collapsing the remaining sequenced polynucleotides into the groups of item 27 according to the best match of the sequenced identifier sequence of each sequenced polynucleotide with the identifier sequence of each group of item 27.
32. The method of any one of items 27 to 31, wherein each polynucleotide in the array has an identifier sequence that is different in essentially every other polynucleotide of the array.
33. The method of item 32, further comprising grouping sequenced polynucleotides that were amplified from the same polynucleotide of the array, wherein the method comprises
   (a) using the percentage determined in item 27(c) to determine a first cut-off for discarding polynucleotide sequences comprising more than the determined first cut-off number of nucleotide blocks in the sequenced identifier sequence that are not correctly sequenced as having one of the pre-selected nucleotide block sequences, and/or to determine a second cut-off for assigning sequenced polynucleotides comprising more than the determined second cut-off number of nucleotide blocks in the sequenced identifier sequence that are not correctly sequenced as having one of the pre-selected nucleotide blocks sequences into different groups instead of the same group; and
   (b) collapsing the sequenced polynucleotides, or the remaining sequenced polynucleotides, into the groups based on sequence identity across the identifier sequences and using the first and/or second cut-off determined in step (a).
34. The method of any one of items 27 to 33, wherein sequenced polynucleotides are a library of polynucleotides generated using the method of item 24 or item 25 or is a library of polynucleotides according to item 26.
35. The method of 34, wherein the grouping of item 33(b) further uses sequence identity across a part of the polynucleotide sequence copied from the analyte and/or the analyte capture region and/or the BC sequence.
36. A method of analysing a library of polynucleotides generated using the method of item 24 or item 25 or a library according to item 26, the method comprising
   (a) obtaining sequencing data for each polynucleotide of the library, including the BC and/or UMI;
   (b) determining the percentage of the identifier sequences of the sequenced polynucleotides that are correctly sequenced as consisting only of one of the pre-defined nucleotide block sequences at each nucleotide bock position;
   (c) using the percentage determined in item 27(c) to determine a first cut-off for discarding polynucleotide sequences comprising more than the determined first cut-off number of nucleotide blocks in the sequenced identifier sequence that are not correctly sequenced as having one of the pre-selected nucleotide block sequences, and/or to determine a second cut-off for assigning sequenced polynucleotides comprising more than the determined second cut-off number of nucleotide blocks in the sequenced identifier sequence that are not correctly sequenced as having one of the pre-selected nucleotide blocks sequences into different groups instead of the same group; and
   (d) collapsing the sequenced polynucleotides, or the remaining sequenced polynucleotides, of the library into groups based on sequence identity across the identifier sequences and using the first and/or second cut-offs determined in step (c).
   polynucleotides, of the library into groups based on sequence identity across the identifier sequences and using the first and/or second cut-offs determined in step (c).

The disclosure will now be described in more detail, by way of example and not limitation, and by reference to the accompanying drawings. Many equivalent modifications and variations will be apparent, to those skilled in the art when given this disclosure. Accordingly, the exemplary embodiments of the disclosure set forth are considered to be illustrative and not limiting. Various changes to the described embodiments may be made without departing from the scope of the disclosure. All documents cited herein, whether supra or infra, are expressly incorporated by reference in their entirety.

The present disclosure includes the combination of the aspects and preferred features described except where such a combination is clearly impermissible or is stated to be expressly avoided. As used in this specification and the appended items, the singular forms “a”, “an”, and “the” include plural referents unless the content clearly dictates otherwise. Thus, for example, reference to “a polynucleotide” includes two or more such polynucleotides.

Section headings are used herein for convenience only and are not to be construed as limiting in any way.

### DESCRIPTION OF THE FIGURES

FIG. 1—Developing a strategy to error correct barcode and UMI sequences from droplet-based sequencing. a The synthesis of our oligonucleotide using dimer blocks of nucleotides. b The cell barcode assignment strategy. c The UMI deduplication strategy. d Simulated data showing the number of barcodes recovered with increasing simulated sequencing error rates. e, f Simulated data showing the difference and coefficient of variation between the deduplicated UMIs and the ground truth. Deduplication was performed using a basic directional network-based approach and accounting for sequencing errors within the paired nucleotides.

FIG. 2—Error correction of both Illumina and Nanopore droplet based scRNA-seq data. Human HEK293T and mouse 3T3 were mixed at a 1:1 ratio and approximately 500 cells were taken for cDNA synthesis. Barcodes and UMIs identified as having at least one sequencing error were processed using before a and after barcode error correction b and the proportion of mouse and human UMIs shown in the barnyard plot. Insert bar plots show the number of cells identified for each species. c The length of the input cDNA nanopore library, as measured using a tapestation. d The read length of the sequenced nanopore library. e Identification of a polyA tail and barcode/UMI within the nanopore reads. f Identification of unambiguous and ambiguous reads based on the nucleotide pairing complementarity. Also shown is the percent recovered ambiguous reads. g A barnyard plot showing the expression of mouse and human UMIs. The insert bar plot shows the number of cells recovered for each species. h UMAP plot of the nanopore isoform expression showing human, mouse or mixed human and mouse cells.

FIG. 3—Nanopore droplet based scRNA-seq identifies isoform diversity. NCI-H929, DF15 and JJN3 myeloma cell lines were mixed at a 1:1:1 ratio and approximately 500 cells were taken for cDNA synthesis. UMAP plot of gene expression a and transcript isoform expression b. c Principal CD74 (HLA-DR) splice variants showing all protein coding transcripts. UMAP plot showing the isoform expression of detected CD74 (HLA-DR) transcripts ENST00000377775.7 d, ENST00000353334.10 e and ENST00000009530.12 f.

FIG. 4—Error correcting Illumina scRNA sequencing data. A dual oligonucleotide scRNA-seq library was generated and 500 human HEK293T and mouse 3T3 cells were sequenced using the Illumina platform. Barcodes that contained a sequencing error, as determined by dual nucleotide block complementarity were identified. Barcodes were then error corrected using increasing edit distances. a The relationship between the number of unique pseudoaligned reads compared to the number of pseudoaligned reads following barcode error correction with increasing Levenshtein distance. b The number of human cells identified using increasing Levenshtein distance for barcode error correction. c The corresponding numbers of mouse cells identified with increasing Levenshtein distance. d, e, f, g, h, i Barnyard plots showing mouse and human UMIs detected per cell.

FIG. 5—Error correcting Nanopore scRNA sequencing data. A dual oligonucleotide scRNA-seq library was generated and 500 human HEK293T and mouse 3T3 cells were sequenced using the Oxford Nanopore platform. Barcodes that contained a sequencing error, as determined by dual nucleotide block complementarity were identified. Barcodes were then error corrected using increasing edit distances. a The number of human cells identified using increasing Levenshtein distance for barcode error correction. b The corresponding numbers of mouse cells identified with increasing levenshtein distance. c, d, e, f, g, h Barnyard plots showing mouse and human UMIs detected per cell.

FIG. 6—The sequencing quality of nanopore reads. The quality of the fastq sequencing reads identified during the nanopore sequencing run were evaluated before a and after b polyA tail identification.

FIG. 7—Implementing a dual nucleotide UMI deduplication strategy improves the recovery of UMIs. A UMI correction method using the original UMI-tools directional network-based deduplication strategy a and one in which the error rate of the dual nucleotide was accounted b.

FIG. 8—Removal of low-quality cells from the NCI-H929, JJN3 and DF15 nanopore sequenced scRNA-seq dataset. Cells expressing greater than 600 UMIs and 100 features per cell was used as a threshold to filter poor quality cells from our 500-cell mixed myeloma cell line dataset. The number of cells before a and after b filtering. The relationship between the number of UMIs and the number of genes before c and after d filtering. A histogram of the number of UMIs before e and after f filtering. The number of counts, features and UMIs across all filtered cells g and across each myeloma cell type.

FIG. 9—The expression of Immunoglobulin Kappa and Lambda constant transcripts. UMAP plot showing the expression of IGKC a, IGKV3D-15 b, IGLV2 c and IGLL5 d.

FIG. 10—A typical droplet based sequencing bead design. The oligonucleotide synthesis takes place in the 5′ to 3′ direction and includes a barcode that is specific for each bead, a polyA capture site and a Unique Molecular Identifier (UMI) that is specific for each captured transcript.

FIG. 11—A dual RNA DNA capture bead, synthesized in the 3′ to 5′ direction. The oligonucleotide contains a barcode that is specific for each bead and a Unique Molecular Identifier (UMI), which are flanked by PCR handles. There is an oligonucleotide sequence on the 3′ end, which is typically a poly T sequence to capture polyA RNA, and an oligonucleotide sequence at the 5′ end, which is typically a DNA capture sequence (e.g. Transposed DNA). The oligonucleotide shown also contains a photocleavable linker at the 3′ end and can also include a hairpin sequence that contains a Uracil base that acts as a cleavage site for APE-1 and UDG enzymes.

FIG. 12—Overview of protocol for dual RNA DNA sequencing library preparation. 1. The oligonucleotide is released from the bead using either a photocleavable linker or a combination of both photocleavable linker and UDG/APE-1 enzymes. 2. RNA and/or DNA are hybridized to the oligonucleotide, for example via RNA polyA tail and known sequences added to transposed DNA. RNA provides template for reverse transcription and Template switch. Captured DNA is ligated to the capture oligo. 3. 1^{st} round of PCR amplification using sequences against the PCR handles, Template switch oligo and transposed MEDS DNA sequence. 4. The PCR amplified product is purified and a second round of PCR performed a. to amplify specifically the DNA from captured RNA, and b. to amplify the captured DNA.

FIG. 13—Tapestation traces show a final library produced for: A. Normal drop-seq using published EZ Macosko-2015 method for performing droplet based sequencing; B. PC drop-seq, as for normal drop-seq but a photocleavable linker is included at the 5′ end of the sequence; and C. PC+HP dual oligo, as described in Example 1.

FIG. 14—UMAP plots showing the number of cells captured by A. Normal drop-seq; B: PC drop-seq; and C: PC+HP dual beads. Each point represents one cell.

FIG. 15—Tapestation trace shows both a DNA library produced from the 5′ capture and an RNA library produced from the 3′ capture. A. Nucleosome phasing is seen following ATAC of HEK293T cells. This is a positive control and confirms that the ATAC protocol generated DNA fragments. B. Shows a final ATAC DNA amplified library following encapsulation and PCR amplification. C. Shows a final post PCR amplified captured RNA product following encapsulation, reverse Transcription and PCR amplification.

### DETAILED DESCRIPTION OF THE INVENTION

### Identifier Sequences

The invention relates to polynucleotides that comprise at least one identifier sequence, such as a barcode sequence or unique identifier sequence, as described further herein. An identifier sequence is a sequence tag that is added to a polynucleotide, typically to indicate the source of the polynucleotide or copies thereof. In some cases, an identifier sequence may be added to a polynucleotide used to capture analytes in a sample, for example when generating a library of sample analytes. An identifier sequence allows polynucleotides having a shared source to be identified. Typical examples of a shared source include the polynucleotides associated with a single micro-particle or micro-bead, or a single well or discrete position on a surface, a sample or part of a sample contacted with the same, or a single capture event between a polynucleotide and an analyte. An identifier sequence may also be added to polynucleotides according to the present invention to determine an amplification and/or sequencing error rate.

The identifier sequences of the invention find particular use when it is desirable to generate a diversity of sequence identifiers for tagging different samples, whilst increasing the ability to distinguish between different identifier sequences, particularly when errors may be introduced during amplification and/or sequencing. The identifier sequences of the invention are built up from nucleotide blocks. The identifier sequence of an individual polypeptide may comprise or consist of any combination of the same or different nucleotide blocks within the limitations described herein. However, the ability to differentiate between identifier sequences used in different polynucleotides according to the present invention is improved by using a pre-determined and limited number of different nucleotide block sequences, either across the full identifier sequence or at each same or equivalent position of the identifier sequence. The sequence of each block can be compared across the different polynucleotides, i.e. because they are at the same known or otherwise identifiable position in each polynucleotide. These pre-defined nucleotide block sequences may be referred to herein as a nucleotide block pool or nucleotide block sequence pool.

All of the nucleotide block sequences within the pre-defined nucleotide block pool differ from every other nucleotide block sequence within the pool by at least two nucleotide substitutions. This provides a number of advantages. One advantage is that a single nucleotide substitution in one or more nucleotide blocks as a result of an amplification or sequencing error will not directly result in mis-identification or allocation of the sequence. All single substitutions in a nucleotide block can be detected by comparing the sequenced blocks to the known nucleotide block sequence pool. Moreover, since single errors in each nucleotide block can always be detected, the error rate across the whole identifier sequence can be used to determine the overall accuracy of the polynucleotide sequence. This determined error rate can in turn be used to correctly identify a higher proportion of the identifier sequences and/or to identify the correct identifier sequences with higher confidence. Importantly, it is not necessary that the full sequence of individual identifier sequences are known ab initio in order to identify amplification/sequencing errors. Hence, the invention allows for diversity in the identifier sequences coupled with error detection and improved identifier sequence identification.

According to the present invention, the same pre-determined nucleotide block pool is used at the equivalent position in an identifier sequence across an array of polynucleotides. Any one of the nucleotide blocks from the pool can be used at a given position in the identifier sequence of any one polynucleotide. The same or different pre-determined nucleotide block pools may be used at other positions within the identifier sequence. This may in part depend on the method used to generate and synthesise the identifier sequence. If the identifier sequence is generated by repeated rounds of degenerate polynucleotide synthesis from a single mixed pool of nucleotide bocks, then generally the whole identifier sequence will be generated from the same single mixed pool of nucleotide block sequences. Hence, in some cases the same pre-determined nucleotide block pool may be used to generate all of the nucleotide blocks of the identifier sequence of the polynucleotides.

The sequence of each the nucleotide blocks is otherwise not particularly limited unless otherwise provided herein and provided that the different nucleotide block sequences can be distinguished when sequenced.

In some cases, the nucleotide block sequences of any one or more pools of nucleotide blocks used to generate an identifier sequence of the invention are selected such that each nucleotide block sequence differs from each other nucleotide block sequence in the pool by at least three nucleotide substitutions. In this case, any nucleotide block comprising any two single nucleotide substitutions can still be identified. This may be particularly useful when using a very low accuracy sequencing and/or amplification method.

The term “nucleotide substitution” as used herein typically means the replacement of a nucleotide at a single position in a longer nucleotide sequence with a different nucleotide at the same position. For example, the nucleotide block sequences ‘AC’ and ‘CT’ are considered to differ by two nucleotide substitutions because the nucleotide in the first position of the nucleotide block sequence has been replaced, and the nucleotide in the second position of the nucleotide block sequence has been replaced. Similarly, the nucleotide block sequences ‘AC’ and ‘CA’ are considered to differ by two nucleotide substitutions because the nucleotide in the first position of the nucleotide block sequence has been replaced, and the nucleotide in the second position of the nucleotide block sequence has been replaced A nucleotide block is typically two or three nucleotides in length. Longer sequences can also be used, for example up to 4, 5, 6, 7, or 8 nucleotides. Pre-synthesised nucleotide blocks used to generate the identifier sequence, as described elsewhere herein, may in some cases include additional nucleotides or spacers that are not part of the identifier sequence. Once inserted into the polynucleotide, these additional nucleotides provide additional sequence elements in between the nucleotide blocks of the identifier sequence, but are not part of the identifier sequence as described herein.

In some cases, all of the nucleotide blocks used in the identifier sequence have the same length. This may be particularly useful when the nucleotide blocks are added using degenerate polynucleotide synthesis, as described further elsewhere herein, where a number of the nucleotide blocks are added consecutively to each polynucleotide. Hence, using a pool of blocks each with the same number of nucleotides ensures that the start and end position of each consecutive nucleotide block in each identifier sequence added to different polynucleotides can be identified. In other cases, different lengths of nucleotide blocks may be used to build up the identifier sequence, particularly when the length used at each position in each identifier sequences added to different polynucleotides is the same, pre-determined or otherwise known. In some cases, for example, the nucleotide blocks may be added using split-and-pool synthesis. In this case, the same length nucleotide block will typically be added to all of the polynucleotides at the same block position, across different pools. However, different sized nucleotide blocks could be added in different rounds of the split and pool synthesis and/or at different block positions.

An identifier sequence may comprise at least 2, more typically at least 3, 4, 5, 6, 7 or 8 nucleotide blocks and up to 12, 13 or 14 nucleotide blocks or more, more typically 6 to 14, 7 to 13, 10 to 14, 13 to 14, 7 to 9, or 8 or 12 nucleotide blocks. The nucleotide blocks are added to or present in the relative polynucleotide at successive, consecutive or non-consecutive block positions. The identifier sequence is defined by the sequence of the nucleotide block at each position. In general, identifier sequences comprising more nucleotide blocks will provide a greater diversity of possible identifier sequences. Hence, the number of nucleotide blocks chosen will be influenced by the total diversity or number of different possible identifier sequences that are needed for a particular purpose. Specific examples are provided in relation to BC and UMI sequences elsewhere herein.

The total diversity of possible identifier sequences is also dependent on the number of nucleotide block sequences that are permitted at each block position in the identifier sequence, i.e. the size of the nucleotide block sequence pool for each position. A pool of di-nucleotide block sequences using only the four canonical nucleotides A, G, T and C (or U) could comprise 2, 3 or 4 different sequences (to ensure that each sequence differs from every other sequence by at least two nucleotide substitutions). A similar pool of tri-nucleotide block sequences using only the four canonical nucleotides may comprise up to 12 different sequences. A greater diversity of nucleotide block sequences could be generated by using longer nucleotide blocks (i.e. 4 or more nucleotides in length) and/or by including non-canonical nucleotides and/or nucleotide analogues, as long as the nucleotides and/or nucleotide analogues use in the sequence can be adequately distinguished when correctly sequenced.

The identifier sequences may in some cases be synthesised using pre-synthesised blocks, as described elsewhere herein. One advantage of this is that more diversity in the identifier sequence can be generated per round of synthesis and using just the same limited pool of nucleotides, for example just the canonical nucleotides A, G, T and C (or U), i.e. by including a larger pool of different nucleotide block sequences as described above.

Hence, the total diversity of possible identifier sequences according to the present invention is determined by (i) the number of nucleotide blocks included in the identifier sequence; and (ii) the number of different nucleotide block sequences included in the pool of sequences that can be used at each block position. If the same nucleotide block pool is used at every block position, then the total possible diversity of sequences is equal to [the number of pool nucleotide blocks]×[the number of nucleotide blocks in the identifier sequence]. For some applications, it is desirable to design the identifier sequence such that the total possible diversity of sequences is the same as, or in excess of the number of polynucleotides, or the number of sub-arrays of polynucleotides that the identifier sequence is intended to distinguish. In some cases, the total diversity of possible identifier sequences is at least 10, or at least 20, 50, 100, 200 or 500 times in excess of the number of polynucleotides in the array or the number of sub-arrays.

In some cases the nucleotide blocks are used consecutively to form a single longer nucleotide block corresponding to the full identifier sequence, i.e. a series of consecutive nucleotide blocks. The term “consecutive” is used to refer to sequential nucleotides blocks in a polynucleotide which immediately follow the previous nucleotide block without intervening nucleotides. For example, an identifier sequence comprising 6 nucleotide blocks, wherein the nucleotide blocks are selected from di-adenosine, di-guanosine, di-cytidine, di-thymidine and di-uridine may have the sequence “AAGGCCTTAAGG”.

In other cases, one or more spacers or other sequence elements may be included between the nucleotide blocks that make up the identifier sequence. The position or identity of the nucleotide blocks of the identifier sequence in the polynucleotide may be determined by any suitable means, for example by adding the nucleotide blocks at pre-determined positions in the polynucleotide or using nucleotide analogues in or otherwise marking or tagging the nucleotide blocks of the identifier sequence. The identifier sequence or the region of the polynucleotide containing all of the nucleotide blocks of the identifier sequence, optionally with other intervening sequence elements, may in some cases be up to 24, 26, 28, 30, 25, 40, 45, 50, 70, 100, 200 or 500 nucleotides or more in length.

In some cases, one or more or each of the nucleotide block pool consists of two or more of the same nucleotide. For example, the nucleotide block pool may in some cases comprise or consist of the blocks AA (di-adenosine), TT (di-thymidine), GG (di-guanosine) and CC (di-cytidine) (or UU (di-uridine)) or/or the blocks AAA (tri-adenosine, TTT (tri-thymidine), GGG (tri-guanosine) and CCC (tri-cytidine) (or UUU (tri-uridine)); or the blocks AAA, TTT, CCX and GGY, wherein X is A, or otherwise T or G, and Y is C, or otherwise A or T; or any combination thereof. In other cases one or more or each nucleotide block sequence may comprise a duplicate or triplicate or other multiple of a nucleotide analogue.

The term “nucleotide” as used herein, particularly in relation to the nucleotide blocks of an identifier sequence, may refer to natural or canonical nucleotides (i.e., “naturally occurring” or “natural” nucleotides), which include adenosine, guanosine, cytidine, thymidine and uridine, or to a non-canonical nucleotide or a nucleotide analogue. The polynucleotides or nucleotide blocks described herein may comprise any combination of natural nucleotides. Alternatively, any non-canonical nucleotides or nucleotide analogues may appear in one or more identifier sequence of a polynucleotide only.

Generally, a nucleotide analogue contains a nucleic acid analogue, a sugar and a phosphate group, or variants thereof, and integrates into a polynucleotide chain in place of a natural nucleotide. Using nucleotide analogues in the identifier sequence may be particularly useful where the nucleotide analogues produce a more distinct signal when sequencing. The person skilled in the art is able to select appropriate nucleotide analogues or combinations of nucleotides/analogues to use in the identifier sequence. Examples of nucleotide analogues that may be included in the polynucleotides or nucleotide blocks described herein are as follows.

Peptide nucleotides, in which the phosphate linkage found in DNA and RNA is replaced by a peptide-like N-(2-aminoethyl)glycine. Peptide nucleotides undergo normal Watson-Crick base pairing and hybridize to complementary DNA/RNA with higher affinity and specificity and lower salt-dependency than normal DNA/RNA oligonucleotides and may have increased stability. Locked nucleotides (LNA), which comprises a 2′-O-4′-C-methylene bridge and are conformationally restricted. LNA form stable hybrid duplexes with DNA and RNA with increased stability and higher hybrid duplex melting temperatures. Propynyl dU (also known as pdU-CE Phosphoramidite, or 5′-Dimethoxytrityl-5-(1-Propynyl)-2′-deoxyUridine,3′-[(2-cyanoethyl)-(N,N-diisopropyl)]-phosphoramidite). An unlocked nucleotide (UNA), which is an analogue of a ribonucleotide in which the C2′-C3′ bond has been cleaved. U-NA form hybrid duplexes with DNA and RNA, but with decreased stability and lower hybrid duplex melting temperatures. LNA and UNA may therefore be used to finely adjust the thermodynamic properties of the polynucleotides in which they are incorporated. Triazole-linked DNA oligonucleotides, in which one or more of the natural phosphate backbone linkages are replaced with triazole linkages, particularly when click chemistry is used for synthesising the polynucleotide. A 2′-O-methoxy-ethyl base (2′-MOE), such as 2-Methoxyethoxy A, 2-Methoxyethoxy MeC, 2-Methoxyethoxy G and/or 2-Methoxyethoxy T. A 2′-O-Methyl RNA base. A 2′-fluoro base, such as fluoro C, fluoro U, fluoro A, and/or fluoro G. Other specific examples of nucleotide analogues that may be used include 2-Aminopurine, 5-Bromo dU, deoxyUridine, 2,6-Diaminopurine (2-Amino-dA), Dideoxy-C, deoxyInosine, Hydroxymethyl dC, Inverted dT, Iso-dG, Iso-dC, 5-Methyl dC, 5-Nitroindole, 5-hydroxybutynl-2′-deoxyuridine (Super T) and 8-aza-7-deazaguanosine (Super G). Super T 2,6-Diaminopurine (2-Amino-dA) and/or 5-Methyl dC. A biotinylated nucleotide. In some cases a polynucleotide, nucleotide block or identifier sequence described herein may comprise at least two, or at least 3, 4, 5, 6, 7, 8, 9, 10, 12, 14, 16, 18, 20, 25, 30, 35, 40, or up to 2%, 5%, 10%, 15%, 20%, 25%, 30%, 35%, 40% or more nucleotide analogues and/or biotinylated nucleotides, or any one type of nucleotide analogue as described herein.

### Methods of Adding an Identifier Sequence to a Polynucleotide

In some cases, the invention relates to a method of adding one of more identifier sequences as described herein to a polynucleotide or to an array of polynucleotides. Polynucleotides used for capturing analytes in a sample to produce a library typically comprise two identifier sequences, a BC sequence and a UMI sequence, as described further herein. One or both of the BC and UMI sequences may be synthesized using the method of the invention.

The method comprises using pre-synthesised nucleotide blocks. The pre-synthesised nucleotide blocks are used to add the nucleotide blocks that make up an identifier sequence as described herein during polynucleotide synthesis/elongation. The pre-synthesised blocks have the same characteristics as the nucleotide blocks of the identifier sequence as described herein, but are (suitable) for use in elongating a polynucleotide during synthesis. The same pool of pre-synthesised nucleotide blocks may be used to add all of the nucleotide blocks that make up an identifier sequence, or the same or a different pool of pre-synthesised nucleotide blocks may be used at each block position of the identifier sequence. The pool of pre-synthesised blocks used to add each block have a pre-determined/selected and/or limited number of different sequences, wherein each nucleotide block sequence in the pool differs from each other nucleotide block sequence by at least two (or more) nucleotide substitutions.

Any suitable method of polynucleotide synthesis/elongation as known in the art may otherwise be used to add the pre-synthesized nucleotide blocks to generate the identifier sequence(s). In some cases, the method comprises degenerate polynucleotide synthesis using a mixed pool of pre-synthesised nucleotide blocks, for example as described elsewhere herein. In some cases, the method comprises “split-and-pool” polynucleotide synthesis for example as described elsewhere herein. In some cases, both methods may be used sequentially, in either order or orientation (5′ to 3′ direction or a 3′ to 5′ direction, depending on the direction of polynucleotide synthesis). For example, degenerate polynucleotide synthesis may be used to add a UMI sequence and split and pool synthesis to add a BC sequence. Where more than one identifier sequence is used, such as a BC sequence and a UMI sequence, they may be added consecutively/adjacently or with other sequence elements or spacers in between the identifier sequences.

The polynucleotide(s) or sub-array(s) of polynucleotides to which the identifier sequence(s) are added may have any other suitable sequence elements or other features, for example as described elsewhere herein. In some cases the identifier sequence is added to each of at least 10, or at least 12, 20, 24, 48, 100, 200, 500, 1000, 2000, 5000, 10⁴, 10⁵, 10⁷, 10⁸, 10⁹ or 10¹⁰ polynucleotides, for example between 200 and 10¹², or between 10², 10³, 10⁴ or 10⁵ and 10¹¹ polynucleotides. In some cases, the method adds a different identifier sequence to at least 10, or at least 20, 50, 100, 200, 500, 1000, 2000, 5000, 10⁴, 10⁵, 10⁶ or 10⁷ different polynucleotides. In some cases, a different identifier sequence is added to each polynucleotide or sub-array of polynucleotides nucleotides to which the identifier sequence is added. In other cases, the method may add the same identifier sequence to more than one polynucleotide. For example, if both a BC sequence and a UMI sequence is to be added to each polynucleotide, wherein the BC sequence identifies each of a number of sub-arrays of the polynucleotides, then the UMI sequence may be designed (i.e. to have sufficient total possible sequence diversity) such that essentially every polynucleotide of the same sub-array receives a different UMI sequence, but the same UMI sequence may be added to two or more polynucleotides that are in different sub-arrays. Such polynucleotides receiving the same UMI sequence can be distinguished based on their combined BC and UMI sequences. The skilled person is able to design suitable identifier sequences and strategies for adding the identifier sequences for their specific purpose.

The pre-synthesised nucleotide blocks may be nucleotide phosphoramidites, such as homodimers, heterodimers, homotrimer or heterotrimer (reverse) amidites, or other suitable pre-synthesised nucleotide blocks suitable for incorporation into a polynucleotide chain for the purpose of the present invention.

### Barcode Sequences (BC)

A barcode sequence is used to identify a group of polynucleotides, or analyte that was captured by a group of polynucleotides, that were initially isolated as an array or sub-array of polynucleotides. All of the polynucleotides of the same initially isolated array/sub-array have the same barcode sequence. Hence, the barcode sequence allows a mixed pool of polynucleotides from different (sub-)arrays to be identified. For example, different polynucleotides may be shared between different micro-beads/micro-particles, or different wells or discrete pre-defined positions on a surface or the like. Each of the polynucleotides associated with each micro-bead, micro-particle, well, or position is a sub-array of the polynucleotides. All of the polynucleotides of a sub-array have the same barcode as each other, and a different barcode to the polynucleotides associated with each other sub-array. Typically each sub-array is contacted with a different sample or part of a sample, as described further elsewhere herein. For example, each sub-array or micro-particle might be contacted with the analytes of different single cells, or each sub-array of each well or position of a surface be contacted with a different part of a tissue sample laid over the surface. The barcode of the capture-polynucleotides tag the analytes from the sample. After analyte capture, the polynucleotides from the different sub-arrays can be combined for amplification and/or sequencing on mass. The barcode sequence associated with each sequenced polynucleotide can subsequently identify the sample or source of the corresponding captured analytes.

In some cases, the barcode sequence of an array or sub-array of polynucleotides as described herein may comprise at least three different nucleotides, or in other cases at least four different nucleotides.

Barcode sequences may be added to polynucleotides using any suitable method known in the art. For example, barcode sequences may be synthesised using a split-and-pool method. In split and pool synthesis an array of polynucleotides, or sub-arrays thereof, are split into groups. Typically four groups are used, but any suitable number of groups may be used. Typically each group includes approximately the same number or proportion of the polynucleotides or sub-arrays. Each group is combined with a different sub-pool of the pre-synthesised nucleotide blocks according to the present invention, wherein the pre-synthesised nucleotide blocks of each sub-pool have different nucleotide block sequences. Typically the nucleotide blocks of each sub-pool have the same nucleotide length. Polynucleotide synthesis within each group adds a pre-defined number, typically one, pre-synthesised nucleotide block from the sub-pools to the polynucleotides of each respective group i.e. in separate reactions or reaction vessels. The polynucleotides are then isolated from the sub-pools of nucleotide blocks. In some cases, the polynucleotides from the different groups are mixed together or re-pooled. The mixed groups may then be essentially randomly re-divided into new groups for a new round of synthesis. In other cases, the groups are re-configured with different combination of polynucleotides or sub-arrays in each new group, without mixing, for example by re-grouping sub-arrays arranged on a surface in different combinations.

Repeat split-synthesis-pool or split-synthesis-regroup cycles creates diversity in the identifier sequences of different polynucleotides or sub-arrays. However, sub-arrays of polynucleotides that remain together as they move between the successive split and pooled groups (for example, all of the polynucleotides bound to a single micro-bead) each obtain the same barcode sequence that can later be used to identify polynucleotides of, or multiplied from, the same sub-array.

As an example, 12 rounds (cycles) of split-and-pool synthesis using 4 groups in each round, wherein each sub-pool comprises a single nucleotide block sequence, would results in 4¹² (16,777,216) possible different barcode sequences. Hence, in a typical experiment using ˜150,000 sub-arrays of polynucleotides (wherein each sub-array is bound to a separate micro-bead, or associated with a different well or discrete position on a surface, for example), the polynucleotides of every sub-array can be certain to have a barcode sequence that is different from essentially every other sub-array. In general, uses that require more uniquely identifiable sub-arrays of polynucleotides require a greater barcode sequence diversity and hence longer barcode sequences and/or larger nucleotide block sequence pools (e.g. more groups/sub-pools per found of synthesis when using a split-and-pool method). A typical barcode sequence may include at least 3, 4, 5, 6, 7, 8, 9, 10, 11 or 12 nucleotide blocks. For example, a barcode sequence may consist of about 6 to 14, 10 to 14, or 11 to 13 nucleotide blocks where each nucleotide block pool comprises four different sequences (for example, where each nucleotide block consists of two natural nucleotides), or may, for example, consist of about 4 to 9, 5 to 8, or 6 to 7 nucleotide blocks where each nucleotide block pool comprises twelve different sequences (for example, where each nucleotide block consists of three natural nucleotides). The barcode sequence is typically synthesised using a number of rounds of split-and-pool synthesis corresponding to the number of nucleotide blocks in the barcode sequence.

Identifier sequences having many of the same sequence characteristics and technical advantages as those described herein could alternatively be achieved using additional rounds of traditional split-and-pool synthesis to add single nucleotides instead of pre-synthesized blocks. However, this would substantially increase the time and cost of manufacture, while also reducing the yield.

### Unique Molecular Identifier Sequence (UMI)

A unique molecular identifier sequence (UMI) is a sequence that can be used to distinguish polynucleotides copied and amplified from an individual polynucleotide. When UMIs are included in an array of polynucleotides used to capture analytes and generate analyte libraries, the UMI typically identifies analyte that was captured by a single polynucleotide and distinguishes analyte that was captured by different polynucleotides in the same array or sub-array.

In some cases, a UMI sequence may be different across essentially every polynucleotide of an initial array (or sub-array) of polynucleotides. In other cases there may be some duplication of UMI sequences in the polynucleotides of an array or sub-array. This might typically be the case for capture-polynucleotides used to capture analyte in a sample, as described further elsewhere herein. As long as the number of unique UMI sequences, or the potential UMI sequences that may be added when a (sub-)array of capture-polynucleotides is being synthesised, is well in excess of the number of capture events and/or the number of different analytes that are present and may be captured in a sample, then the UMI will still uniquely identify essentially each polynucleotide that captures analyte in the sample.

In some cases at least 10%, or at least 20%, 30%, 40%, 50%, 60%, 70%, 80% of the UMI sequences in an array or sub-array of polynucleotides is a unique sequence that is not shared with any of the other UMI sequences of the (sub)-array. In some cases the maximum number of repeats of any one UMI sequence in an array or sub-array is less than 10%, or less than 7%, 5%, 2%, 1%, 0.5%, 0.2%. 0.1%, 0.05%, 0.02%. 0.01% of the total number of polynucleotides in the (sub-)array.

Barcode sequences (BC) and UMIs are often used together, with the BC sequence identifying and distinguishing a plurality of sub-arrays of polynucleotides (all polynucleotides of the same sub-array share the same BC) and the UMI sequence identifying and distinguishing separate polynucleotide in each sub-array. Hence, different polynucleotides in different sub-arrays may share the same UMI, but the BC sequence and UMI together may uniquely identify each polynucleotide of an initial array, or each polynucleotide of an initial array that captures analyte, and may be used to identify subsequent copies.

The UMI can be used to distinguish between copies of a polynucleotide arising from (i) a single capture of analyte by a single polynucleotide, and (ii) multiple captures of different copies of the same analyte on different capture polynucleotides. Hence, a UMI may be used to digitally count analytes in a sample and detect duplicate sequences derived from a single capture event.

UMIs may be added to polynucleotides using any suitable method known in the art. One efficient method for adding UMI sequences to an array of polynucleotides is using multiple rounds of degenerate synthesis in the presence of a mixed pool of different pre-synthesised nucleotide blocks.

The potential diversity of UMI sequences is dependent on the number of nucleotide blocks that make up the sequence and the number of different nucleotide block sequences in the nucleotide block sequence pool(s), as described above. The number of nucleotide block sequences and the number of blocks per UMI will typically be selected to ensure that the potential sequence diversity is in excess of the number of polynucleotides in the relevant (sub-) array or the number of expected capture events, or the number of different capture analytes. In some cases, the total diversity of possible UMI sequences is at least 10, or at least 20, 50, 100, 200 or 500 times in excess.

As an example, eight rounds of degenerate synthesis using a pool of four different nucleotide block sequences will generate a UMI diversity of 4⁸ (65,536). For a typical sub-array of around 10⁷ polynucleotides (for example, the polynucleotides bound to a single micro-bead), this diversity would be sufficient to ensure that a different UMI sequence is added to most of the polynucleotide associated with the same sub-array, and essentially every polynucleotide subsequently that captures analyte. However, shorter or longer UMI sequences could be used for (sub-)arrays comprising fewer or more polynucleotides. Alternatively, increased diversity could be achieved by using a larger pool of polynucleotide block sequences. For example, a similar total diversity to the example above could be achieved in four to five rounds of degenerate synthesis using a pool of twelve different nucleotide block sequences (total possible diversity=12⁴ (20,736) using 4 rounds of synthesis, or 12⁵ (248,832) using 5 rounds of synthesis). Hence, the UMI sequence may in some cases include at least 3, 4, 5, 6, 7, 8, 9, 10, 11 or 12 nucleotide blocks, for example, about 6 to 10, or 7 to 9 nucleotide blocks, or 8 nucleotide blocks.

### Polynucleotides

The invention relates to polynucleotides comprising an identifier sequence as described herein. The terms “polynucleotide”, “oligonucleotide” or “oligo” may in some cases be used herein interchangeably, and refer to a string of nucleotide monomers in a chain typically linked by phosphodiester bonds. As used herein, a polynucleotide is a chain of nucleotides of any length, whilst an oligonucleotide typically comprises up to 50 nucleotides. In some cases the polynucleotides of the invention may be at least 50, or at least 56, 60, 70, 80, 90, 100, 110, 120 or 125 and/or up to 130, 140, 160, 180, 200, 225, 250, 275, 300, 350, 400, 500, 1000 or 2000 nucleotides or more in length, for example between 50 or 56 and 1000, 500, 400, 300 or 200 nucleotides in length. The polynucleotides may be DNA (or single-stranded DNA) or RNA. Polynucleotides have a chemical orientation defined by the position of the linking carbon in the five-carbon sugar of each consecutive nucleotide in the chain. Polynucleotides may be manufactured by the addition of nucleotides at either the 5′ end (manufacture in a 5′ direction) or the 3′ end (manufacture in a 3′ direction) to elongate the chain. Likewise, sequence elements along the length of a polynucleotide have a sequential order defined by the directionality of the chain of nucleotides that is either 5′ to 3′ or 3′ to 5′.

In some cases, the invention relates to an array, or isolated set, of polynucleotides. Each polynucleotide of the array comprises at least one identifier sequence as described herein. In some cases, every polynucleotide of the array, or a sub-array thereof, has a different identifier sequence, such as a UMI sequence that uniquely identifies each polynucleotide in the (sub-)array, optionally in combination with a barcode sequence. In other cases, a (sub-)array of polynucleotides may all comprise the same identifier sequence, i.e. a barcode sequence, which identifies each polynucleotide as belonging to, or having been copied/amplified from, that (sub-) array. The polynucleotide array may comprise a plurality of sub-arrays, wherein the barcode sequence of each sub-array is different from the barcode sequence of each other sub-array.

In some cases the array may comprise at least 10, or at least 12, 20, 24, 48, 100, 200, 500, 1000, 2000, 5000, 10⁴, 10⁵, 10⁷, 10⁸, 10⁹ or 10¹⁰ polynucleotides, for example between 100 and 10¹², or between 200, 50, 10³, 10⁴ or 10⁵ and 10¹¹, or between 10⁶, 10⁷, 10⁸, or 10⁹ and 10¹⁰ polynucleotides. In some cases, the array of polynucleotides is divided into at least 10, or at least 12, 20, 24, 48, 100, 200, 500, 1000, 2000, 5000, 10⁴, 10⁵, 10⁶ or 10⁷ different sub-arrays, as described herein. A typical sub-array may comprise at least 10, or at least 12, 20, 24, 48, 100, 200, 300, 400, 500, 700, 1000, 2000, 5000, 10⁴, 10⁵, 10⁶, 10⁷, 10⁸, 10⁹ or more polynucleotides, for example between 100 and 10¹², or between 1000 and 10¹¹, or between 10⁴ and 10¹⁰ polynucleotides per sub-array.

The polynucleotides according to the invention are typically single-stranded, though portions of the polynucleotide may be double stranded, for example when a capture-polynucleotide is bound to or hybridized to a sample analyte.

In some cases, the polynucleotides described herein further comprise one or more analyte capture sequences, and/or a polymerase chain reaction (PCR) handle sequence and/or one or more cleavable linker, or any combination thereof. In some cases, the polynucleotides are analyte capture-polynucleotides that may be used to capture analytes in a sample, for example to generate one or more libraries of analytes of the sample, as further described elsewhere herein. Examples of analyte capture regions include a polythymidine sequence; an (polynucleotide) aptamer; a sequence of at least 10 nucleotides for hybridising to a target polynucleotide analyte; a biotinylated nucleotide sequence; or an ATAC-med sequence. In some cases it can be useful to include a further (known) sequence in between an identifier sequence and an analyte capture region, such as a polythymidine, to ensure that the identifier sequence and the analyte capture region can be clearly distinguished. For example, in some cases the additional sequence may comprise or consist of the sequence “ACGCACGC” The sequence elements of the capture-polynucleotides (analyte capture regions, PCR handles, linkers, identifier sequences and so on) are generally arranged such that copies of the polynucleotide sequence that tag any captured analytes (e.g. at the 3 and/or 5′ ends of the capture-polynucleotides) include the relevant identifier sequences (e.g. UMI and/or BC sequences). The identifier sequences allow the tagged analytes to be identified as having been captured by a particular capture-polynucleotide or originating from a particular sample or digitally counted, as described further elsewhere herein.

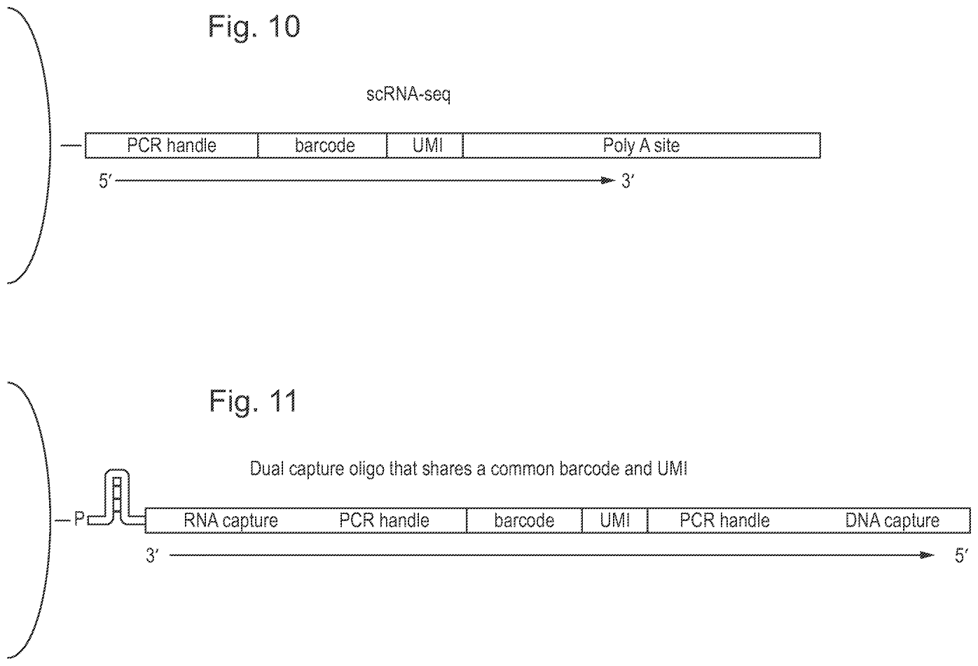

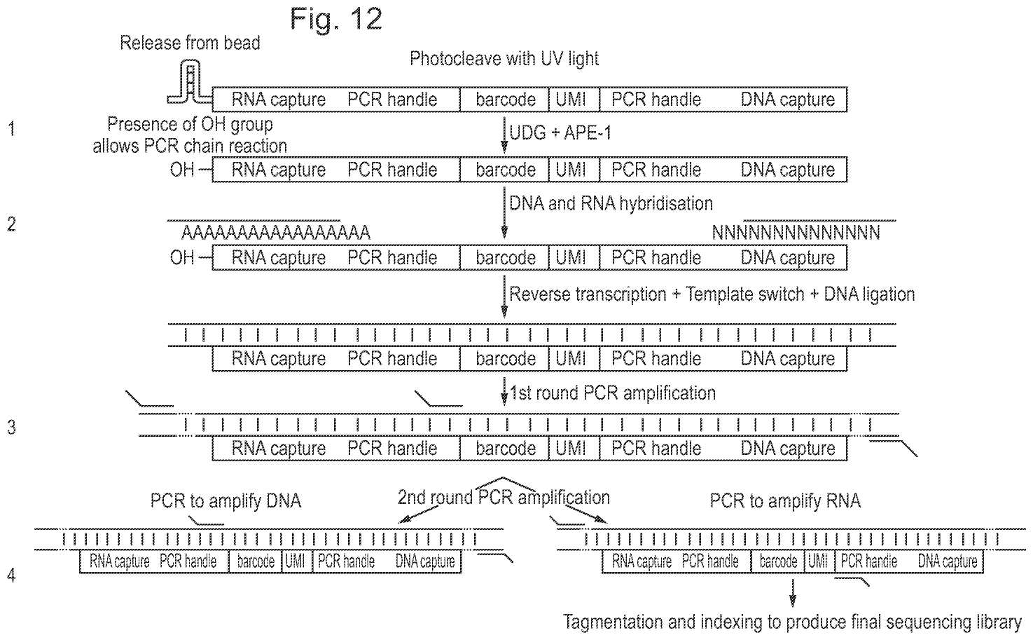

In some cases the polynucleotides may comprise the following sequence elements in a 5′ to 3′ direction: (a) a PCR handle sequence; (b) at least one identifier sequence, optionally a BC sequence and/or a UMI sequence, in either order or orientation; and (c) an analyte capture region. This design is typical for polynucleotides synthesized in a 5′ to 3′ direction on a solid support such as a micro-bead (FIG. 10).

In some cases, the polynucleotides comprise the following sequence elements in a 3′ to 5′ direction: (a) optionally a linker that is cleavable to provide a free 3′ hydroxyl group on the polynucleotide after cleavage; (b) a 3′ end analyte capture region; (c) at least one identifier sequence, optionally a BC sequence and/or a UMI sequence, in either order or orientation; and (d) a PCR handle sequence. In some cases, the polynucleotides comprise the following sequence elements defined in a 3′ to 5′ direction: (a) optionally a linker that is cleavable to provide a free 3′ hydroxyl group on the polynucleotide after cleavage; (b) a 3′ end analyte capture region; (c) a first PCR handle sequence, (d) at least one identifier sequence, optionally a BC sequence and/or a UMI sequence, in either order or orientation; (d) optionally a second PCR handle sequence; and (e) a 5′ end analyte capture region. In other cases, the polynucleotides comprise in a 3′ to 5′ direction: (a) 3′ hydroxyl group; (b) a 3′ end analyte capture region; (c) optionally a first polymerase chain reaction (PCR) handle sequence; (d) at least one identifier sequence, optionally a BC sequence and/or a UMI sequence, in either order or orientation; (e) optionally a (second) PCR handle sequence; and (f) optionally a 5′ end analyte capture region. This type of dual analyte capture bead, synthesized in the 3′ to 5′ direction, is described in GB Application No. 2007059.5. An example is shown in FIG. 11. One advantage of using a polynucleotide synthesized in a 3′ to 5′ direction, particularly in the context of the present invention, is that pre-synthesised 3′ to 5′ blocks, particularly trimer blocks or longer, are less complex and costly to produce than 5′ to 3′ pre-synthesised blocks.

In some special cases, some of the elements may overlap. In particular, in some cases, the 3′ end analyte capture region may partially or fully overlap with the first PCR handle sequence and/or the 5′ end analyte capture region may partially or fully overlap with the second PCR handle sequence.

In some cases, the polynucleotides may comprise non-nucleotide linking elements (i.e. spacers), for example phosphoramidite spacers, such as 17-O-(4,4′-Dimethoxytrityl)-hexaethyleneglycol, 1-[(2-cyanoethyl)-(N,N-diisopropyl)]-phosphoramidite (HEG). For example, a spacer may be included 3′ to the 3′ analyte capture region, or between the 3′ analyte capture region and the bead. The polynucleotides may also comprise further sequence elements in addition to those defined herein. Typically, however, the 3′ analyte capture region is immediately downstream of the 3′ end hydroxyl group produced on cleavage of the linker. Likewise, the 5′ analyte capture region is typically at the 5′ terminus of the polynucleotide.

The polynucleotides of the present invention may in some cases comprise any of the further features described in GB Patent Application No: 2007059.5.

### Linkers

In some cases, the polynucleotides comprise a sequence element that is cleavable to provide a free 3′ hydroxyl group on the polynucleotide after cleavage. Cleavage of the linker may provide a 3′ end analyte capture region, such as a polythymidine for capturing RNA. Cleavage can also be used to separate a polynucleotide that has been synthesised on a solid support, such as a micro-bead, from the support. This can simplify subsequent processing steps, e.g. in the production of a library of analytes captured by the polynucleotides.

In some cases, the sequence is a linker that is light (e.g. UV light)-sensitive (photocleavage), that is temperature-sensitive or thermolabile (thermocleavage), or that is cleaved on chemical exposure (chemical cleavage). In other cases, the linker is a sequence element that is cleavable by an enzyme or combination of enzymes to provide a free 3′ hydroxyl group. In one example, the enzymes may be a DNA glycosylase such as a uracil-DNA glycosylase (UDG), and a class I AP endonuclease, such as APE-1. The glycosylase excises a base from the polynucleotide sequence to create an apurinic/apyrimidinic (AP) site, and the endonuclease nicks the phosphodiester backbone leaving a 3′ hydroxyl group. The linker may comprise a uracil. The uracil may be immediately 3′ to the start of the 3′ end analyte capture region, such as a polythymidine region. The linker may comprise a double-stranded region, such as a hairpin structure immediately 3′ to and ending at or including the cleavage site, for example immediately 5′ to a uracil. In another example, the enzyme may be an endonuclease IV, such as the double-strand-specific Escherichia coli endonuclease IV (Nfo) described in Levin et al. (1988, J. Biol. Chem. 263: 8066-8071) and Pirpenburg et al. (2006, PLoS Biol. 4(7): 1115-1121).

In some cases, enzymatic cleavage of polynucleotide to provide a free 3′ end hydroxyl group may be more efficient after release of the polynucleotide from bead. Accordingly in some cases, the polynucleotide comprises, in a 3′ to 5′ direction, a first cleavable linker, such as a photocleavable, thermocleavable, chemically cleavable or enzymatically cleavable linker, and a second linker that is cleavable to provide a free 3′ hydroxyl group on the polynucleotide after cleavage. In one example, the polynucleotide may comprise, in a 3′ to 5′ direction, (a) a first cleavable linker, (b) a double stranded region, optionally comprising or followed by a uracil, and (d) the 3′ end analyte capture region, such as a polythymidine. In a specific example, the polynucleotide comprises the sequence HEG-AAAAAAGGCGC-HEG-GCGCCU.

Other examples of enzyme that may be used to provide a 3′ hydroxyl group on polynucleotide after cleavage include restriction enzymes or an esterase. Accordingly, in some cases the linker comprises a suitable restriction site or an ester linkage. If the corresponding restriction enzyme cuts double stranded DNA, then the linker may comprise a double-stranded (or hairpin) region comprising the restriction site. In other cases the linker does not comprise a restriction enzyme cleavage site and/or does not include an ester linkage.

Restriction enzymes having longer recognition sites cleave fewer off-target sample polynucleotides. One example restriction enzyme with a seven base pair recognition sequence is SapI (Type IIS restriction enzyme). In some cases the linker may comprise a double stranded region comprising the SapI recognition site (GAAGAGC . . . GCTCTTC) and optionally an adjacent polyT region as the 3′ analyte capture region. For example, the sequence GAAGAGCT-HEG-AGCTCTTC could replace the region between the polyT and polyA in the polynucleotide described herein in Reference Example 1, with or without including the PCLinker between the double-stranded region and the bead. The restriction site is outside of the recognition site and would cut within the polyT region in the example above, leaving a 3′ polyT region with a free 3′ hydroxyl group.

BspQI, an isoschizomer of SapI, may also be used. However, BspQI needs a higher incubation temperature than SapI and may be less buffer tolerant.

Example restriction enzymes having a six base pair recognition sequence include BspHI, BspEI, MmeI, NruI, XbaI, BclI, FspI, MscI, BsrGI, PsiI-v2, BstBI and DraI (well-known type II restriction enzymes) and BbsI, BeiVI, BmrI, BsaI, EarI & Esp3I (well-known type IIs restriction enzymes). The known recognition and restriction sites of these restriction enzymes may be included in the linker, in a double-stranded/hairpin region as needed, optionally adjacent to a polyT region as the 3′ analyte capture region. For example the BsrGI recognition sequence is T/GTACA . . . T/GTACA. In one example, the sequence T/GTACAT-HEG-AT/GTACA could replace the region between the polyT and polyA in the polynucleotide described herein in Example 1, with or without including the PCLinker between the double-stranded region and the bead.

### Analyte Capture Regions

The analyte capture region(s) may be any nucleotide sequence suitable for capturing analyte in a sample. In some cases the analyte(s) may be biological analytes or may be selected from polynucleotides DNA and/or RNA, or from oligonucleotides, DNA, cDNA, RNA, mRNA, proteins, polypeptides and/or peptides, cell surface receptors or cells. In some cases the analytes may additionally be selected from amino acids, metal ions, inorganic salts, polymers, nucleotides, oligonucleotides, polynucleotides, dyes, bleaches, pharmaceuticals, diagnostic agents, recreational drugs, explosives and/or environmental pollutants. Such analytes may be captured, for example, by an aptamer.

Typically the analyte capture region(s) sequence may be at least 10, or at least 15, 20, 25 or 30 nucleotides in length, such as from about 15 to about 50, from about 20 to about 40 or from about 25 to about 35 nucleotides. In some cases, the analyte capture region(s) may comprise one or more nucleotide analogues, such as analogues described herein, that form double-stranded hybrids with higher stability than natural nucleotides. In this case, the analyte capture region(s) could be shorter, such as at least 3, 4, 5, 6, 8 or 9, for example between 3 and 50, or 40 or 30 or 20 nucleotides in length, provided that the analyte capture region(s) was capable of hybridizing to target analyte such that analyte sequence can be amplified as described herein. One or both analyte capture regions may include nucleotide analogues as described herein.

In some cases, an analyte capture region may be a DNA capture region, an RNA or mRNA capture region, or a polypeptide capture region.

In one example, an analyte capture region, particularly the 3′ analyte capture region may be a polythymidine. Polythymidine may hybridise to and capture any polynucleotide in the sample that comprises a suitable polyadenosine, such as polyadenylated mRNA. Typically the polythymidine may be at least 10, or at least 15, 20, 25 or 30 thymidines in length, such as from about 15 to about 50, from about 20 to about 40 or from about 25 to about 35 thymidines. When the polynucleotide is attached to bead, the polythymidine may be immediately to the 3′ side of the cleavage site of the linker that provides the free 3′hydroxyl group after cleavage. When polynucleotide is not attached to bead, the polythymidine comprises the hydroxyl group at the free 3′ end of the polynucleotide.

In other cases, the analyte capture region(s) may comprise or consist of an aptamer. Aptamers can be produced using SELEX (Stoltenburg, R. et al., (2007), Biomolecular Engineering 24, p 381-403; Tuerk, C. et al., Science 249, p 505-510; Bock, L. C. et al., (1992), Nature 355, p 564-566) or NON-SELEX (Berezovski, M. et al. (2006), Journal of the American Chemical Society 128, p 1410-1411). Typically, an aptamer may be at least 15 nucleotides in length, such as from about 15 to about 50, from about 20 to about 40 or from about 25 to about 30 or nucleotides in length. An aptamer may bind to analyte such as small molecules, proteins, nucleic acids or cells. Aptamers may be designed or selected to bind to pre-determined target analyte(s). In one example, the aptamer may bind to a Coronaviridae protein or SARS-CoV-2 protein, such as any of the SARS-CoV-2 structural protein sequences provided herein.

In some cases, an analyte capture region(s) may comprise or consist of a biotinylated nucleotide sequence. Nucleotides or polynucleotides may be biotinylated using methods known in the art. Typically the biotinylated sequence may be at least 10, or at least 15, 20, 25 or 30 nucleotides in length, such as from about 15 to about 50, from about 20 to about 40 or from about 25 to about 35 nucleotides. A biotinylated capture region may be used to capture any suitable target analyte comprising streptavidin or avidin.

In some cases, the analyte capture region(s) may comprise or consist of a nucleotide sequence designed to hybridise to a complementary sequence in a target polynucleotide analyte. In some cases, the capture region is for capturing/hybridising to transposed DNA. In this case an analyte capture region may comprise or consist of a sequence that is complementary to transposed DNA in a sample, for example to a transposed MEDS DNA sequence. In some cases, the sequence may be gene or transcript-specific, such as a polynucleotide sequence that is complementary to, or at least 80%, 85%, 90%, 95%, 98% or 99% complementary to, a viral sequence, a bacterial sequence or a sequence associated with a disease or disorder, such as a sequence from a cancer-associated antigen or a neoantigen. In some cases, the analyte capture region(s) may hybridise to a nucleotide sequence that encodes a part of a Coronaviridae protein or SARS-CoV-2 protein, such as any of the SARS-CoV-2 structural protein sequences provided herein.

In other cases, the sequence may be designed to capture a polynucleotide tag added to analyte of interest.

In some cases, 5′ analyte capture region may be absent or may comprise any of the characteristics described herein for the 3′ analyte capture region. Typically, however, the 5′ analyte capture region does not consist of a polythymidine sequence because mRNA hybridised to polythymidine at the 5′ analyte capture region cannot be converted to cDNA by reverse transcription from the 5′ end.

In some cases, the 3′ and 5′ analyte capture regions may be for binding the same type of analyte. For example, both regions may be DNA analyte capture regions. In other cases, both ends may be biotinylated and bind to analyte comprising streptavidin or avidin, or both regions may be protein capture regions, and/or both ends may comprise an aptamer. In some cases, the 3′ and 5′ analyte capture regions may be for binding different types of analyte. For example, in some cases the 3′ analyte capture region be for binding RNA, for example the 3′ analyte capture region be a polythymidine, and the 5′ analyte capture region may be for binding DNA or protein, such as any types of DNA or protein described herein. In a different example, both analyte capture regions may comprise different sequences for hybridising to complementary sequence in different polynucleotide analytes.

In some cases, all of the polynucleotides of the array may have the same 3′ and/or 5′ analyte capture regions. In other cases, different 3′ and/or 5′ capture regions may be used. For example, the 3′ and/or 5′ capture regions of the array could comprise different aptamers or may have different sequences for hybridising to different sequences in DNA analytes. In some cases, the 3′ and/or 5′ analyte capture regions may consist of at least two, or at least 3, 4, 5, 10, 20, 50, 100 or 200 different polynucleotide sequences. In some cases, the 3′ or 5′ analyte capture region may be the same in each polynucleotide of the array, but the other analyte capture region may have different sequences amongst the polynucleotides of the array.

In some cases, some of the polynucleotides in the array may be bound to analyte at the 3′ and/or 5′ end as described herein.

In some cases, the bound analyte comprises a polynucleotide sequence that is complementary to, or at least 10%, 20%, 30%, 40%, 50%, 60%, 70%, 80%, 85%, 90% 95%, 98%, 99% complementary to, the polynucleotide sequence of the analyte capture region, and the complementary polynucleotide sequence of the analyte sequence is hybridised to the analyte capture region.

In some cases, at least about 0.0005%, or at least about 0.001%, or 0.005%, or 0.01% or 0.05% or 0.1% of polynucleotides in the array may be bound to or hybridised to analyte. Where the polynucleotides comprise both a 3′ analyte capture region and a 5′ analyte capture region, some of the polynucleotides may bind or hybridise to one analyte via the 5′ analyte capture region and to a second analyte via the 5′ analyte capture region. In some cases, at least about 0.00001%, or at least about 0.0001%, or 0.001%, or 0.01%, or 0.1% of polynucleotides in the array may be bound to or hybridised to analyte at both ends of the polynucleotide (polynucleotides having both a 5′ and a 3′ analyte capture region).

In some cases, the same type of analyte may be bound or hybridised at both ends of polynucleotide. For example, both the 3′ analyte capture region and the 5′ analyte capture region may be bound to DNA, or to protein. In some cases, analyte may bind to the 3′ and 5′ analyte capture regions sequentially. In some cases, for example, mRNA may bind to a 3′ polythymidine analyte capture region and reverse transcription reaction produces an RNA/cDNA hybrid at the 3′ end. The 5′ analyte capture region may subsequently capture a different analyte.

### PCR Handle Sequences

A PCR handle sequence hybridizes to PCR oligonucleotide primers during a PCR reaction. Typically a PCR handle sequence may be at least 15, 16, 17, 18, 19 or 20 nucleotides in length and/or up to and 21, 22, 23, 24, 25, 30 or 35 nucleotides in length, for example about 15 to 30, or 18 to 25 nucleotides. In some cases, the PCR handle sequence(s) may comprise one or more nucleotide analogues, such as analogues described herein, that form double-stranded hybrids with higher stability than natural nucleotides. In this case, the PCR handle sequence(s) could be shorter, such as at least 3, 4, 5, 6, 8, 9, 10, 11, 12, 13 or 14 nucleotides in length, provided that the PCR handle sequence(s) was capable of hybridizing to PCR oligo as described herein. One or both PCR handle sequence(s) may include nucleotide analogues as described herein.

In some cases the PCR handle sequences of each polynucleotide in an array, or associated with the same microbead or microparticle, may be the same. In other cases, difference sequences may be used. In some cases, the polynucleotides may have a first and a second PCR handle sequence as described herein. The first and second PCR handle sequences may, in some cases, have the same or complementary sequences. In other cases, different or non-complementary sequences are used. In some cases, the invention relates to a plurality of microbeads or microparticles. In some cases, the (first and/or second) PCR handle sequences of the polynucleotides of each of the microbeads or microparticles are the same as the (first and/or second) PCR handle sequences of essentially each other microbead or microparticle. In some cases one or more of the PCR handle sequences comprises or consists of one of the following sequences: 5′-GTGGTATCAACGCAGAGTAC-3′; 5′-GTCCGAGCGTAGGTTATCCG-3′ The PCR handles may be compatible with standard Illumina sequencing to eliminate the need for custom read 1 sequencing primers to read BC/UMI.

### Sub-Arrays

Sub-arrays of polynucleotides as described herein are typically used to probe different parts or sub-elements of a sample, such as individual cells of a cellular sample or different spatial positions on a surface of a tissue sample. Different capture-polynucleotides of the same sub-array may capture different analytes in the same part or sub-element of the sample. Typically the capture-polynucleotides of each sub-array are separated from other sub-arrays before being contacted with the analytes of the part/sub-element of the sample. For example, each sub-array may be isolated and/or contacted with the analytes in a separate fluidic compartment as described further herein.

### Micro-Particles

In some embodiments the invention relates to micro-particles. The micro-particles comprise a micro-bead and an (sub-)array of polynucleotides as described herein. Microbeads are typically less than 500 μm, or less than 400 μm, 300 μm or 200 μm in diameter, for example, between 10 and 500 μm, 20 and 400 μm, 40 and 300 μm, or 50 and 200 μm. Typically a micro-bead is approximately spherical or sphere-like. Micro-beads with surface-attached polynucleotides are well-known in the art and may be made from, for example, a biocompatible polymer such as polystyrene, polyacrylamide or hydroxylated methacrylic polymer, or from controlled pore glass. The micro-particles described herein may comprise a micro-bead as described herein with an array of polynucleotides as described herein, wherein each polynucleotide in the array is attached to the bead at the 3′ or the 5′ end of the polynucleotide. The opposite end (5′ or 3′) is typically free in solution.

Dissolvable beads and hydrogel beads have also been described and are encompassed in the present disclosure. Dissolvable beads may, for example, be made from crosslinked acrylamide with disulfide bridges that are cleaved with dithiothreitol. An array of polynucleotides may be embedded in the bead matrix and released when the bead is dissolved. A micro-particle comprising a micro-bead and an array of polynucleotides bound to the micro-bead, as described herein, may be a typical micro-bead with surface bound polynucleotide or a dissolvable bead with embedded polynucleotide.

Polynucleotides may be synthesised on the bead using methods known in the art or described herein. For example, the phosphoramidite method may be used, in which one nucleotide or pre-synthesised block is added per synthesis cycle. The identifier sequence(s) may be generated as described elsewhere herein. Other and/or longer sequence elements may in some cases be added using enzymatic ligation methods, such as using DNA ligase, chemical ligation methods, such as phosphoramidate ligation, and/or click chemistry ligation methods, such as the azide-alkyne cycloaddition reaction. Suitable methods are known in the art.

In some cases, the invention relates to a plurality of micro-particles as described herein. The number of micro-particles may be selected for a specific purpose or experiment, such as to capture sample analytes for the generation one or more libraries. Typically the plurality of micro-particles comprises at least 1000, or at least 10,000, or 50,000, or 100,000, or 150,000, for example between 1000, 10,000, or 50,000, or 100,000, or 150,000 and 10⁷, or between 10,000 and 10⁶ or between 50,000 and 600,000, or between 50,000 and 400,000, or between 10,000 or 100,000 and 300,000 micro-particles. Typically the set of polynucleotides associated with each micro-particle have a different shared barcode sequence from the polynucleotides associated with essentially each of the other micro-particle. Hence, the analytes that are captured by the set of polynucleotides associated with each micro-particle can be distinguished. The potential diversity of barcode sequences is dependent on the number of nucleotide blocks that make up the barcode sequence and the size of the nucleotide block pools, as described above. The barcode sequence will typically be long enough that the potential sequence diversity is well in excess of the plurality of micro-particles, typically at least 50× or 100× in excess, or least 10, 20, 50, 100, 200 or 500 times in excess.

### Solid Support-Based Arrays

In some embodiments the invention relates to a surface comprising a plurality of wells or discrete pre-determined positions, wherein each well or discrete pre-determined position is associated with a (sub-)array of polynucleotides as described herein. Typical examples include solid supports such as well plates (for example, one or more 6, 12, 24, 48, 96, 384, 1536 or 3456 well plates) microplates, or slides, as are known in the art.

The polynucleotides may be attached to the surface using chemistry known in the art. The polynucleotide may be attached to the solid support through a linker of non-specific bases. The surface may be made from any suitable materials, for example, a biocompatible polymer such as polystyrene, polyacrylamide or hydroxylated methacrylic polymer, or from controlled pore glass.

In some cases, the polynucleotides may be synthesised on the solid support in a similar way to synthesis on a micro-bead, as described above. In other cases, the polynucleotides may be pre-synthesised and then divided by aliquoting different sub-sets (sub-arrays) of the polynucleotides and/or micro-particles, as described herein, into the wells or onto the pre-defined discrete spatial positions.

Solid-supports or surfaces comprising an array or plurality of sub-arrays of polypeptides as described herein are particularly useful for capturing and/or analysing sample analytes in a spatial manner, for example the analytes of a tissue or other two or three dimensional sample laid over other otherwise contacted with the surface/support in a manner that captures the spatial arrangement of the analytes as captured.

### Kits

In some case the invention relations to a kit for generating one or more libraries from one or more groups of analytes, the kit comprising an array of polynucleotides, a micro-particle, a plurality of micro-particles, or a surface/solid support of the invention as described herein. The invention also relates to the use of a comprising an array of polynucleotides, a micro-particle, a plurality of micro-particles, or a surface/solid support of the invention, or a kit of the invention as described herein for generating one or more libraries from one or more groups of analytes. The kit may also comprise buffers, enzymes and other components used for generating the library as described herein and/or instructions for use in a method of generating the library.

### Analyte Capture and Library Generation

In some embodiments, the invention provides a method for capturing analyte in a sample. The method comprises contacting the sample with a surface/solid support, micro-particle or an array of polynucleotides as described herein and allowing analytes in the sample to bind to the 3′ and/or 5′ analyte capture regions of the array of polynucleotides.

In some cases, the analytes are from a single cell, such as a bacterial cell, or single cell nucleus, single cell vesicle, such as an exosome or mini-vesicle, or other compartment enclosed by a lipid membrane. In some cases, the method comprises isolating the single cell, nucleus, vesicle or other compartment, or a lysate thereof, and contacting the isolated cell, nucleus, vesicle, compartment or lysate with a single micro-particle or array of polynucleotides as described herein.

In some cases, the analytes are from a sample comprising a plurality of cells (such as bacterial, prokaryotic or eukaryotic cells), cell nuclei, vesicles or other compartment enclosed by a lipid membrane, or a two or three dimensional sample such as a tissue sample. Analytes from different cells, cell nuclei, vesicles or other compartments or from different spatial positions in the sample may be captured by separate arrays of polynucleotides as described herein.

The method may in some cases include preparing a single cell or single cell nuclei or vesicle suspension from the sample and/or isolating single cells, nuclei, vesicles or lysates thereof in separate compartments. A single cell, nuclei, vesicle or cell/nuclei/vesicle lysate of the sample and a single micro-particle may be isolated in each of a plurality of separate compartments. In some cases, there may be at least 500, or at least 5,000, or 25,000, or 50,000, or 100,000, for example between 500 and 500,000, or between 2,000 and 200,000, or between 20,000 and 100,000 separate compartments, each comprising a single cell, nuclei or cell/nuclei lysate and a single micro-particle or (sub-)array of polynucleotides as described herein. The method may in in other cases include preparing a two or three dimensional sample for contact with a surface/solid support as described herein.

Micro-particles or (sub-)arrays of polynucleotides may be contacted with sample or analyte in a compartment, typically a fluidic compartment. For example, the compartment may be a well, such as a well in a multi-well plate or microplate or slide, a discrete site/position on a microfluidic chip, or a (micro-)droplet, which may be formed in an oil emulsion. Such compartments may in some cases be made using a microfluidics device as known in the art. In some cases, the sample, a micro-particle, an array of polynucleotides, a cell, cell nuclei or cell/nuclei lysate may be encapsulated or co-encapsulated in a fluidic compartment.

The cell and/or cell nucleus membrane(s) may be lysed, before or after contact with the micro-particle or array of polynucleotides. In some cases, the cell and/or nucleus may be lysed and target polynucleotides may be amplified, for example by PCR, before being brought into contact with the microparticle or array of polynucleotides. The analytes that bind to the 3′ and/or 5′ analyte capture regions may then be include the PCR products.

In some cases, a linker of the polypeptides may be cleaved or a micro-particle hydrogel may be dissolved to separate the polypeptides from (the bead of) a micro-particle or from a surface/solid support before or after contact with the cell, nucleus or lysate, for example by exposing the micro-particle to UV light or heat, or contacting the micro-particle with appropriate chemicals or enzyme(s), such as the enzyme(s) described herein. In some cases, it may be convenient to include the micro-particle or array or polynucleotides in cell/membrane lysis buffer, and/or the cell, nucleus or lysate in a buffer comprising agents needed for cleavage of the linker, such that mixing the two buffers exposes cell/nucleus to lysis buffer and/or micro-particle to the chemical milieu for linker cleavage, as well as bringing the array of polynucleotides into contact with analyte from the cell or nuclei. For example, a microfluidics device may be used to join two aqueous flows into discrete microfluidic droplets. One flow may comprise a single cell or single cell nuclei suspension in cell buffer and optionally chemicals or enzymes needed to cleave the polynucleotide linker. The other flow may comprise a suspension of micro-particles as described herein, optionally in cell/membrane lysis buffer. Some of the droplets that are formed comprise both a cell or nuclei and a micro-particle, resulting in contact between the analytes and the array of polynucleotides.

Other components needed for downstream reactions and processes may be included in the cell/nucleus buffer and micro-particle/lysis buffer or be added to or used to wash the plate or other surface/solid support. In some cases, the cell/nucleus buffer may comprise template switch oligonucleotides. In some cases, the micro-particle/lysis buffer may comprise reverse transcriptase, in particular when the 3′ analyte capture region of the polynucleotides is an RNA capture region, such as a polythymidine.

RNA in the sample may bind to the 3′ RNA capture regions of the polynucleotides and reverse transcription using the bound RNA as template provides an RNA/cDNA hybrid at the 3′ end of the polynucleotide. Template switch oligonucleotides may be added at the end of the RNA/cDNA hybrid. The template switch oligonucleotides and the PCR handle sequence 5′ to the identifier sequence(s) (e.g. BC and/or UMI) provide a pair of PCR handle sequences for PCR amplification of the cDNA/RNA hybrid. PCR amplification may use a pair of oligonucleotide primers that hybridise to the template switch oligonucleotide sequence and the PCR handle sequence 5′ to the identifier sequence(s).

DNA in the sample may bind to a 5′ and/or 3′ DNA capture region of the polynucleotides. DNA captured at the 5′ end may be amplified using oligonucleotide primers that hybridise to a PCR handle sequence on the captured DNA and the complement of the PCR handle sequence 3′ to the identifier sequence(s) (e.g. BC and/or UMI). DNA captured at the 3′ end may be amplified using oligonucleotide primers that hybridise to a PCR handle sequence on the captured DNA and the PCR handle sequence 5′ to the identifier sequence(s).

When the array polynucleotides comprise both 3′ and 5′ analyte capture regions, a first round of PCR amplification using all of the relevant PCR oligonucleotide primers will generate PCR products comprising the identifier (e.g. the barcode and UMI) sequences and polynucleotide sequence from polynucleotide analyte bound to the 3′ or 5′ analyte capture regions. A third PCR product comprising the identifier (e.g. barcode and UMI) sequences, but not any sequence from bound analytes, will also be generated from the pair of primers that hybridise one to the PCR handle sequence 5′ to the barcode and UMI sequences and the other to the complement to the PCR handle sequence 3′ to the barcode and UMI sequences. One or more further rounds of PCR amplification using only one or the other pair of PCR oligonucleotide primers described above eliminates this additional PCR product from further amplification.

After amplification, the PCR products may be sequenced using methods known in the art. Barcoding means that different compartments containing different polynucleotide arrays bound to analyte, or reaction products thereof, may be merged after capture. For example, the different compartments, such as droplets, may be merged after reverse transcription and/or before PCR. Downstream processes may then be carried out in bulk.

PCR product sequences having different barcodes can be digitally assigned to different samples or sample parts or sub-elements (for example cells or cell nuclei), as described herein. Analytes can be digitally counted by using the UMI sequences to identify duplicates derived from the same capture event.

The invention further relates to a method of generating one of more libraries using the products and methods described herein, and to libraries produced by such methods. The methods described herein can be used to generate libraries corresponding to the analytes that bind to the 3′ and/or 5′ analyte capture regions of the polynucleotides described herein. In some cases, the method comprises amplifying polynucleotides, or portions of the polynucleotides, that are captured by a 3′ or 5′ end analyte capture region of a polynucleotide comprising an identifier sequence as described herein. The method may comprise amplifying a polynucleotide comprising an identifier sequence as described herein that captures or otherwise tags a non-polynucleotide analyte of a sample. The method comprises (a) capturing analytes in the sample on an array of polynucleotides synthesised according to a method described herein, an array of polynucleotides as described herein, a micro-particle described herein, a plurality of micro-particles described herein, or a surface described herein; (b) generating copies of the array of polynucleotides, including any sample polynucleotides captured by the array polynucleotides and the BC and/or UMI sequence(s); and (c) amplifying the number of copies of each polynucleotide to produce a library of polynucleotides amplified from or tagging analytes in the sample and including the BC and/or UMI sequence.

The method for generating one or more libraries from one or more groups of analytes may comprise (i) contacting the sample with an array of polynucleotides as described herein; (ii) allowing analytes to bind to the 3′ end and/or 5′ end analyte capture regions of the polynucleotides; and (iii) generating one or more libraries from the analytes bound to the 3′ end and/or 5′ end analyte capture regions, optionally wherein the method comprises generating a first library from the analytes bound to the 3′ end analyte capture regions, and generating a second library from the analytes bound to the 5′ end analyte capture regions.

In some cases, the analyte capture region may bind to RNA in the sample, and the method comprises reverse transcription using the bound RNA as template to provide an RNA/cDNA hybrid. Template switching may be used to extend the end of the RNA/cDNA hybrid to include a template switch PCR handle sequence. The polynucleotide may be amplified via PCR amplification using primers that hybridize to (A) the template switch PCR handle sequence and (B) the PCR handle sequence 5′ to the identifier sequence(s) (e.g. BC and/or UMI sequence).

The 5′ end analyte capture region may bind to DNA in the sample. PCR amplification using a pair of PCR primers that hybridize to (A) a PCR handle on the DNA bound to the 5′ end capture region, and (B) the complement of the PCR handle sequence 3′ to the UMI and BC may be used. The 3′ end capture region may bind to DNA in the sample. Amplification using a pair of PCR primers that hybridize to (A) a PCR handle on the DNA bound to the 3′ end capture region, and (B) the PCR handle sequence 5′ to the identifier sequence(s) (e.g. BC and/or UMI sequence) may be used.

The one or more groups of analytes are from a single cell, single cell nucleus or single vesicle. The single cell or single cell nucleus or single vesicle may be isolated with a single micro-particle in a fluidic compartment, such as a microfluidic compartment. The cell, cell nucleus or vesicle may be lysed. The linker may be cleaved to provide the array of polynucleotides.

The analytes may be from a sample comprising a plurality of cells or cell nuclei or vesicles. A single cell or single cell nuclei or single vesicle of the sample and a single micro-particle may be isolated in each of a plurality of separate fluidic compartments, wherein the polynucleotide array of essentially each micro-particle has a different barcode sequence. The isolated cells, cell nuclei or vesicles may be lysed. The linker of the polynucleotides may be cleaved to provide an array of polynucleotides with free 3′ hydroxyl groups in each fluidic compartment. The methods may further comprise one or more further rounds of PCR amplification. Each round may use a pair of primers that hybridize to the template switch handle sequence(s) and the PCR handle sequence 5′ to the identifier sequence(s) (e.g. BC and/or UMI sequence); or a PCR handle on a DNA analyte and the complement of the first PCT template handle. One or more of the libraries may be sequenced.

The library may in some cases correspond to the analytes from a single cell, cell nuclei or other sample that is contacted with a single micro-particle or array of polynucleotides as described herein, or may correspond to the analytes from a plurality of single cell, cell nuclei or other samples. In some cases, a first library may be generated from the analytes bound to the 3′ analyte capture regions, and a second library may be generated from the analytes bound to the 5′ analyte capture regions, particularly when the 3′ and 5′ analyte capture regions capture different types of analyte as described herein. In this case, barcoding the polynucleotides of each micro-particle or array of polynucleotides as described herein allows matching of the libraries or parts of each library that correspond to analytes from the same cell, nuclei or sample. For example, captured RNA and DNA analytes (or any other two or more analyte types described herein) that are captured by the same array of polynucleotides contacted with the same cell, nucleus or sample may be digitally matched and distinguished from RNA and DNA (or other analytes) captured by other arrays of polynucleotides contacted with other cells, nuclei or samples analysed in the same experiment.

The library or libraries of the invention and the libraries made by the methods of the invention may be made from the analytes of any suitable sample. Specific examples include a sample of cells, cell nuclei or cellular vesicles, a single cell, a single cell nucleus, a single vesicle, a tissue sample or tissue section, or a biological fluid sample, optionally blood, a blood fraction, serum, plasma, saliva or urine sample.

### Library and Identifier Sequence Handling and Analysis

An identifier sequence as described herein may be used according to the present invention to determine the accuracy of a method of amplifying and/or sequencing an array of polynucleotides, in particular a library of polynucleotides of unknown sequence and/or a library of polynucleotides as described herein. The identifier sequences described herein may also be used according to the present invention in an improved method of identifying or grouping copied, amplified and/or sequenced polynucleotides according to their actual polynucleotide sequence or the sequence of the polynucleotide from which they have been copied and/or amplified. Using an identifier sequence according to the present invention, instead of a BC and/or UMI sequence currently used in the art, allows a higher proportion of the polynucleotides comprising the identifier sequence that are copied, amplified and/or sequenced to be correctly identified according to their identifier sequence, despite errors that may have been introduced into the sequence.

The method comprises obtaining sequencing data for each polynucleotide or amplified polynucleotide, including the identifier sequence. Any suitable sequencing method known in the art may be used, including long- and short-read sequencing methods. The percentage of the identifier sequences of the polynucleotides that are correctly sequenced as consisting only of one of the pre-defined nucleotide block sequences at each nucleotide bock position then estimated or determined. This can be done by comparing the obtained sequence in each polynucleotide at each nucleotide block position with the pre-defined pool of nucleotide block sequences at each respective position and determining the percentage of polynucleotides that are a complete match. This percentage is indicative of and may be used to determine the accuracy of the method of amplification and/or sequencing, and/or of the obtained polynucleotide sequences.

The methods described herein may also be used to identify nucleotide blocks that have been sequenced incorrectly or have been amplified incorrectly. A nucleotide block that has been sequenced or amplified incorrectly will have a nucleotide block sequence at the pre-determined position which does not match a nucleotide block sequence from the pre-defined pool of nucleotide block sequences relating to the nucleotide block position.

In some cases, the array of polynucleotides comprises a plurality of sub-arrays as described herein, wherein the polynucleotides of each sub-array have a different identifier sequence (i.e. a barcode sequence).

Sequenced polynucleotides having a “complete match” cross the identifier sequence may be allocated into groups, wherein each group has the same identifier sequence, or the reverse complement of the identifier sequence. Sequenced polynucleotides allocated to the same group/having the same complete match identifier sequence are assumed to have all be sequenced from and/or amplified from polynucleotides of the array having the same identifier sequence.

Once the percentage of “complete match” sequenced identifier sequences has been determined, this value may further be used to improve allocation of the remaining sequenced polynucleotide (i.e. those known to comprise one or more amplification and/or sequencing error) to the groups described above. The percentage of “complete match” identifier sequences can be used to determine a cut-off for discarding from further analysis polynucleotide sequences comprising more than the determined cut-off number of nucleotide blocks in the sequenced identifier sequence that have not been correctly sequenced as having one of the pre-selected sequences. Knowing the percentage of “complete match” identifier sequences/the overall accuracy of the amplification/sequencing allows sequences to be allocated with greater confidence to the correct groups and allows more of the sequenced polynucleotides to be allocated to a group. This improved the efficiency of analyte library generation, validation and analysis, allowing more data to be extracted from the same method of analyte capture and library generation. The remaining polynucleotide sequence that have not been discarded according to the determined cut-off may be further collapsed into the groups described above according to the best match of their sequenced identifier sequence, using methods known in the art. Sequenced polynucleotides allocated to the same group by this method are assumed to have all be sequenced from and/or amplified from polynucleotides of the array having the same identifier sequence, despite errors in the sequence known to have been introduced by the method of amplification and/or sequencing. In other words, variation in the sequence of the polynucleotides that are grouped according to this method is assumed to be the result of replication, amplification and/or sequencing errors.

In some cases, the array polynucleotides are grouped as described above are grouped according to a barcode sequence corresponding to each sub-array of polynucleotides as described above. In some cases, the array polynucleotides may further comprise a separate identifier sequence which is a UMI sequence according to the invention, wherein the UMI sequence of each polynucleotide within each sub-array is different from each other UMI sequence of each other polynucleotide in the same sub-array. In other cases, each polynucleotides of the whole array may comprise an identifier sequence that is different in each polynucleotide (i.e. UMI sequence). The sequenced polynucleotides may be (further) grouped using the method described above according to their sequenced UMI sequences.

The percentage of “complete match” sequences (in relation to the BC sequences and/or the UMI sequences) may also be used in some cases to determine a further (second) cut-off for assigning/allocating any polynucleotide sequences not comprising a “complete match” (and optionally not discarded according to the first cut-off described above) and having more than the determined second cut-off number of nucleotide blocks in the sequenced identifier sequence that are not correctly sequenced as having one of the pre-selected nucleotide blocks sequences into different groups instead of the same group. This is particularly useful when the total number of groups and/or initial polynucleotides in the library of polynucleotides is unknown, which is often the case for UMI sequences resulting from unique analyte capture events, but may be the result of a number of different factors. The number of polynucleotides on a single micro-particle, single cell or well of a plate, or at a single pre-determined position on a surface is difficult to determine, although it may be estimated. Moreover, the number of polynucleotides that capture an analyte may only be a small proportion of the total number of polynucleotides in an array.

The sequenced polynucleotides, or the remaining sequenced polynucleotides, can then be put into groups based on sequence identity across the identifier sequences and using the first and/or second cut-off Sequenced polynucleotides allocated to the same group by this method are assumed to have all be sequenced from and/or amplified from polynucleotides of the array having the same identifier sequence, despite errors in the sequence known to have been introduced by the method of amplification and/or sequencing.

In some cases, the sequenced polynucleotides are a library of polynucleotides generated using a method of the invention or a library of the invention as described herein. Sequenced polynucleotides of the library that have been amplified from the same polynucleotide of the array that has captured an analyte in a sample are expected to share additional sequence across other parts of the sequenced polynucleotide, particularly the uniquely captured polynucleotide analyte. Hence, in some cases polynucleotide sequence identity within a portion of the sequenced polynucleotide that has been copied from the captured analyte may additionally be used together with the UMI sequence to group the sequenced polynucleotides by UMI group (i.e. polynucleotides assumed to have been amplified from the same polynucleotide of the array). In cases where the polynucleotides of the array comprise both a UMI and a BC sequence, polynucleotide sequence identity across the BC sequence can also be used, together with the UMI sequence to group the sequenced polynucleotides by UMI group.

In some cases, the invention relates to a method of analysing a library of polynucleotides generated using the method described herein or a library as described herein. The method may comprise (a) obtaining sequencing data for each polynucleotide of the library, including the BC and/or UMI; (b) determining the percentage of the identifier sequences of the sequenced polynucleotides that are correctly sequenced as consisting only of one of the pre-defined nucleotide block sequences at each nucleotide bock position; (c) using the determined percentage to determine a first cut-off for discarding polynucleotide sequences comprising more than the determined first cut-off number of nucleotide blocks in the sequenced identifier sequence that are not correctly sequenced as having one of the pre-selected nucleotide block sequences, and/or to determine a second cut-off for assigning sequenced polynucleotides comprising more than the determined second cut-off number of nucleotide blocks in the sequenced identifier sequence that are not correctly sequenced as having one of the pre-selected nucleotide blocks sequences into different groups instead of the same group; and (d) collapsing the sequenced polynucleotides, or the remaining sequenced polynucleotides, of the library into groups based on sequence identity across the identifier sequences and using the first and/or second cut-offs determined in step (c).

### EXAMPLES

### Example 1—Barcode and UMI Error Correction

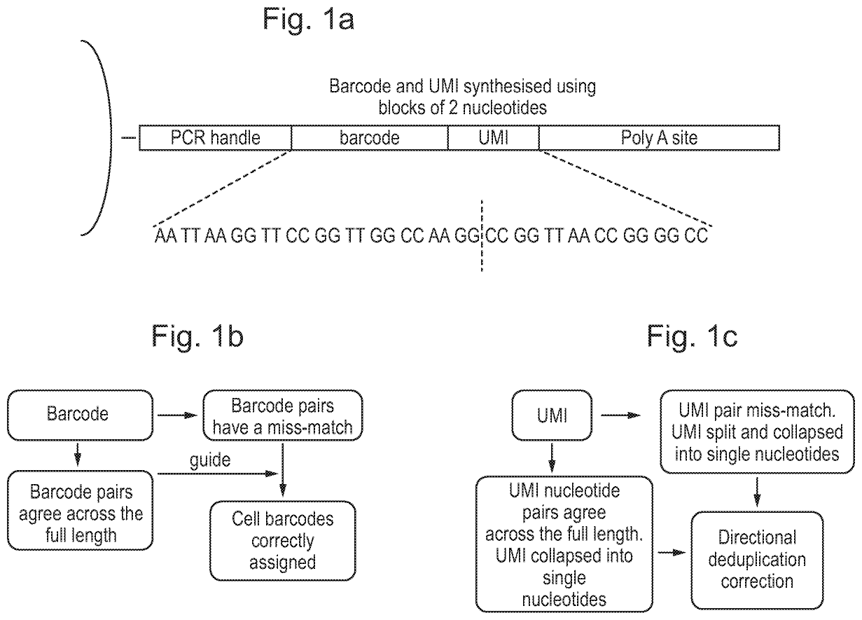

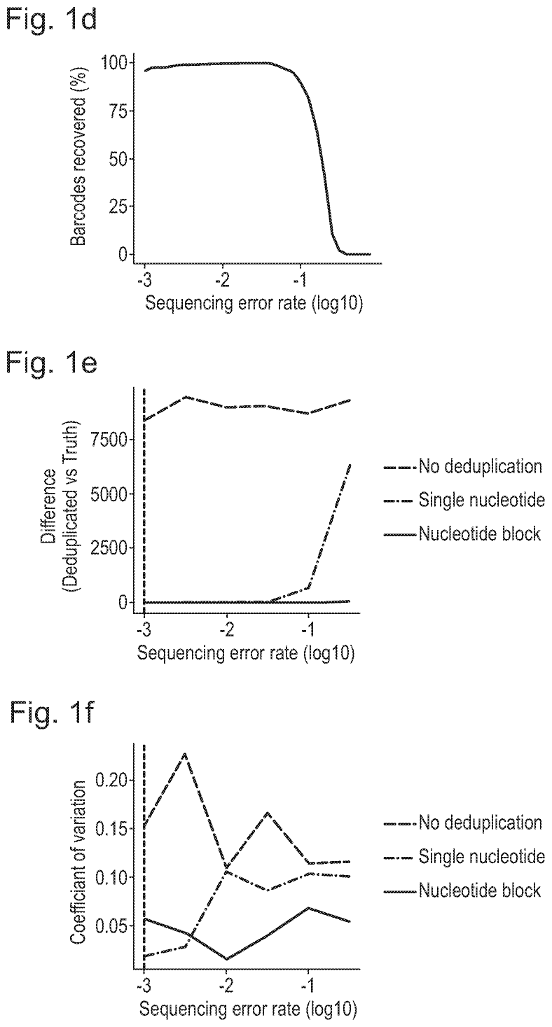

In order to ensure high accurate barcode and UMI assignment, a novel single-cell oligonucleotide synthesis strategy was developed that incorporates homodimer nucleotides into the synthesis reaction (FIG. 1a). This allows for accurate assignment of barcodes to cells that have not undergone either PCR or sequencing errors (FIG. 1b). Additionally, unlike other computational methods for nanopore correction, this approach also allows error correction of the UMI (FIG. 1c).

In order to correctly assign barcodes to cells, a computational strategy was developed in which true barcodes were identified following a two-pass assignment method. Firstly, true barcodes were identified based on nucleotide pair complementarity across the full length of the barcode. Next, these true barcodes were used as a guide to error correct the remaining barcodes. Using simulated data, the strategy is capable of correcting barcodes with a high sequencing error rate, with 96% of barcodes recovered with a sequencing error rate of up to 10% (FIG. 1d).

Next, the directional network based UMI correction method, first proposed by UMI-tools (Smith et al., Genome Res 27 (2017) 491-499), was modified to deduplicate the UMI sequences. In simulated data, single-nucleotide UMIs are ineffectually deduplicated with sequencing error rates >5% using the original UMI-tools implementation. However, using double nucleotide blocks and incorporating the sequencing error rate of the UMI, the UMI sequences were effectively deduplicated, even at sequencing error rate >10%, (FIG. 1e and FIG. 1f).

Accurate Assignment of Cell Barcodes and Unique Molecular Identifiers within Sequencing Data

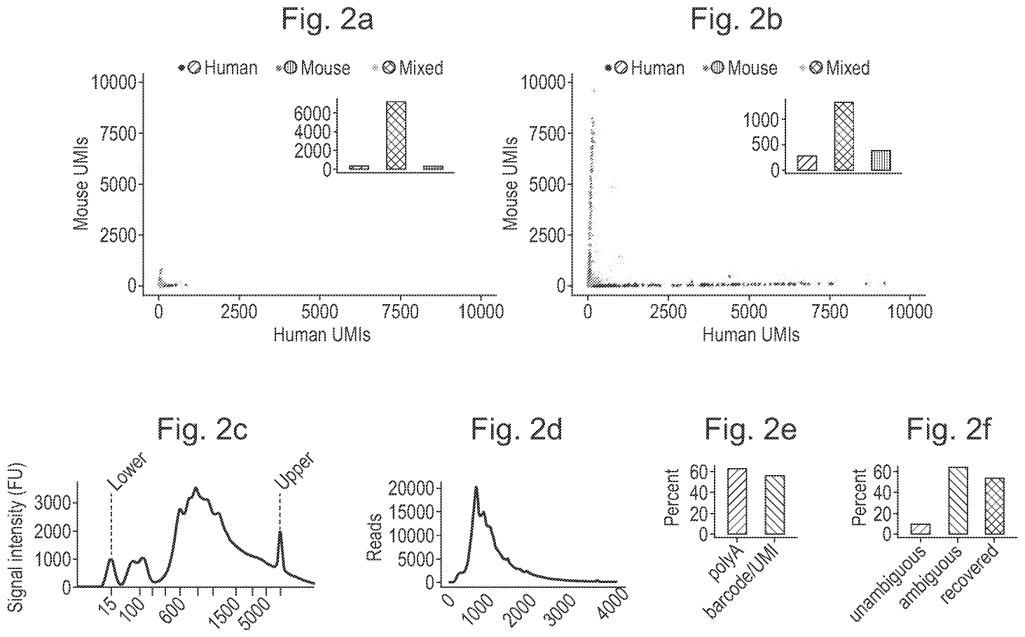

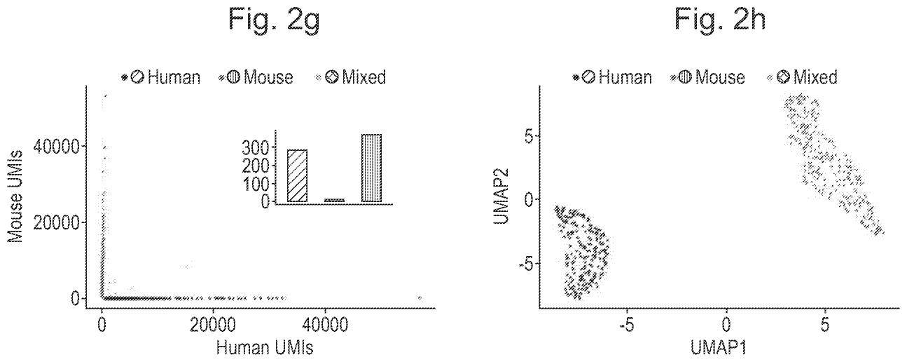

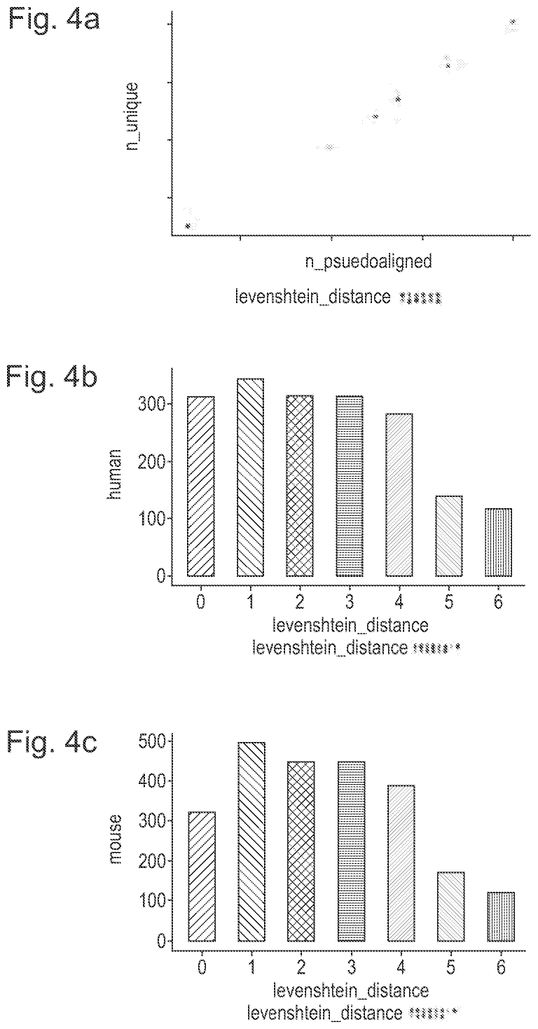

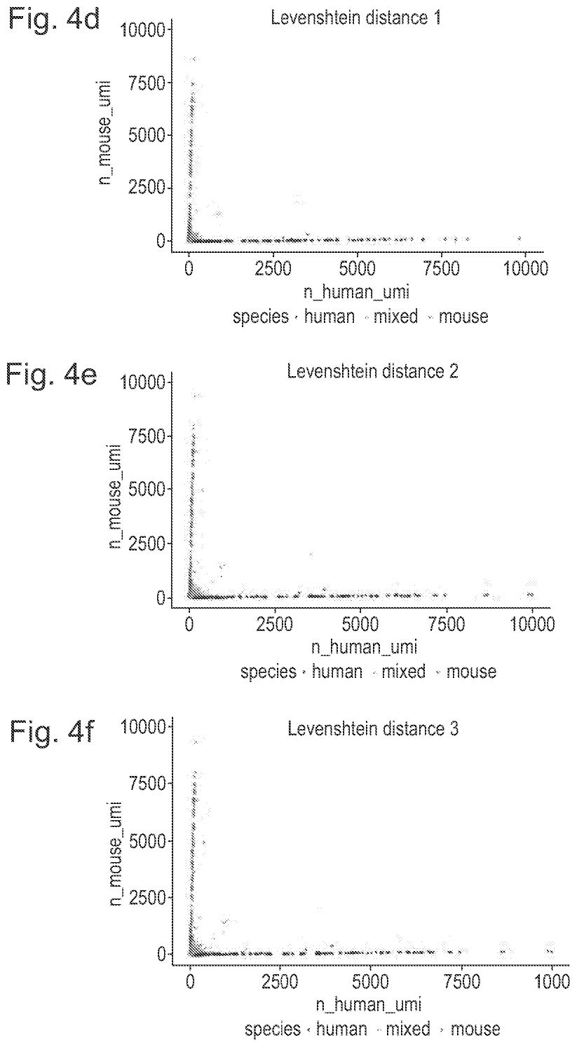

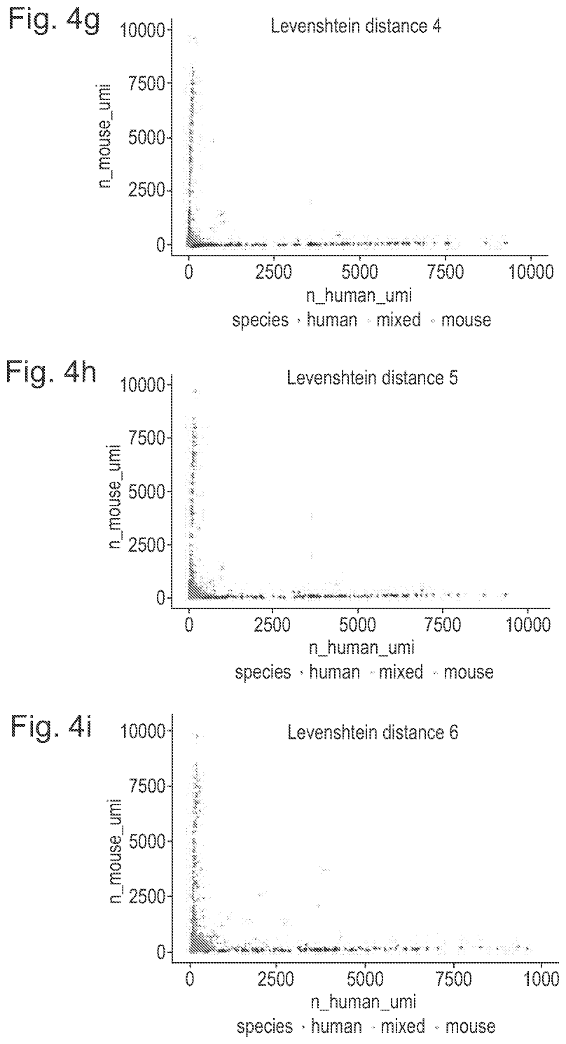

In order to validate the method, a 500 human HEK293T and 500 mouse 3T3 single-cell Dropseq library was prepared using the DolomiteBio Nadia system (Cribbs et al. Proc Natl Acad Sci USA 117 (2020) 6056-6066.). This library was then sequenced using Illumina short-read sequencing. The low sequencing error rate associated with this technology provides a good test bed in which to evaluate the performance of the barcode and UMI correction methodology described herein. Overall, the per base accuracy was 87.75%, with 68% of the total barcodes showing full dual nucleotide block complementarity across the full barcode sequence. Using those perfectly aligned reads, the ability to error correct error sequenced barcodes was evaluated. The performance of the method was evaluated using a series of increasing edit distances between the error sequenced barcodes and the accurately sequenced barcodes (FIG. 4). Analysis of uncorrected error prone barcodes revealed low number of total cells, with the majority of those cells containing a mixed contamination of mouse and human reads (FIG. 2a). Using an edit distance of 4 resulted in accurate assignment of both mouse and human reads (FIG. 2b).

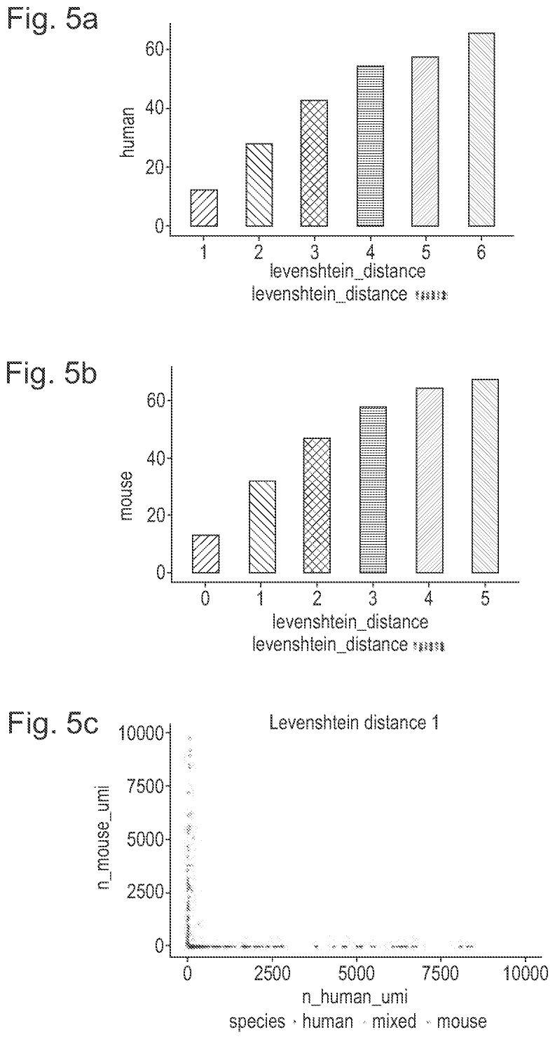

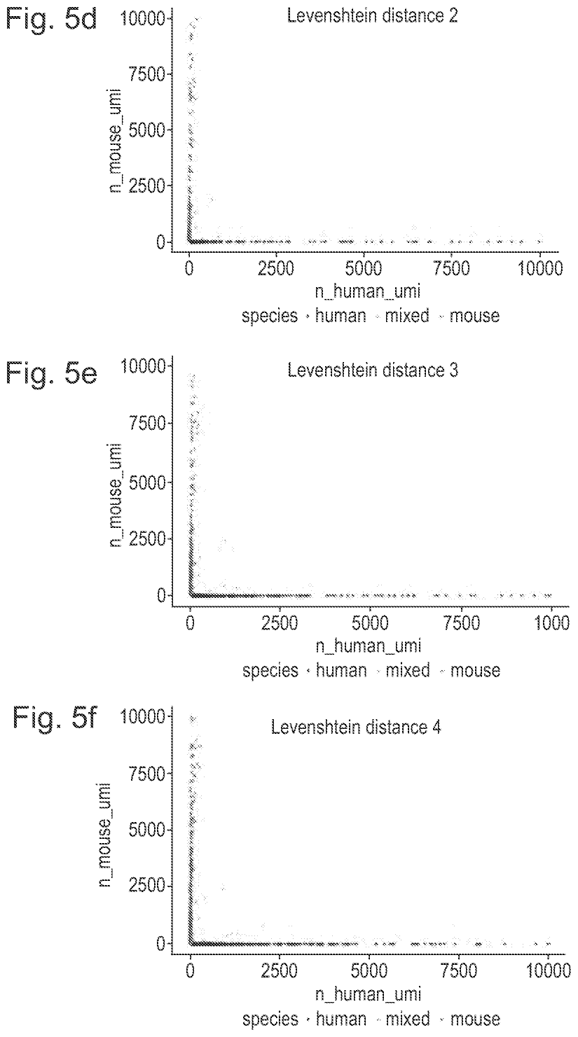

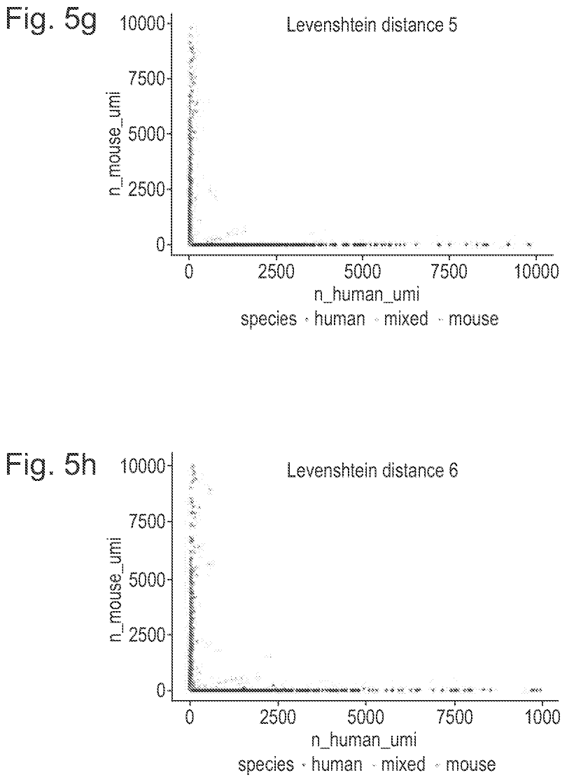

Having demonstrated that the single-cell oligonucleotide design can provide reliable basecalling and barcode error rate information, the technology was next applied to Oxford nanopore sequencing. Using the same cDNA from FIG. 1a and FIG. 1b (FIG. 2c), nanopore sequencing was found to produce a reads distribution of an approximate length to that of the input cDNA (FIG. 2d). The performance of the method was evaluated using a series of increasing edit distances between the error sequenced barcodes. Even up to an edit distance of 6, a substantial proportion of error corrected barcodes could be recovered. However, because of the increasing numbers of mixed human and mouse cells an edit distance of 4 was chosen (FIG. 5). The basecalling accuracy was estimated at 63.5%, with 28.5% of barcodes showing full dual nucleotide complementarity across the full barcode. The polyA and cell barcode error rate is substantially higher than that of Illumina sequencing, but in line with those reported by long-read sequencing with PacBio of 10× Genomics libraries (Gupta et al. Nat Biotechnol (2018)).

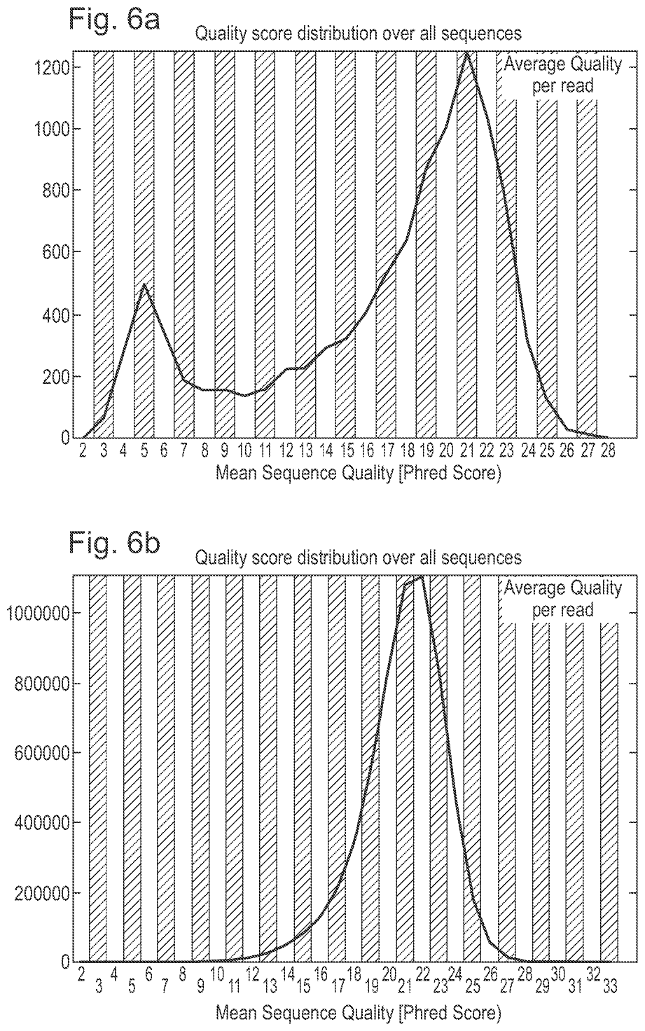

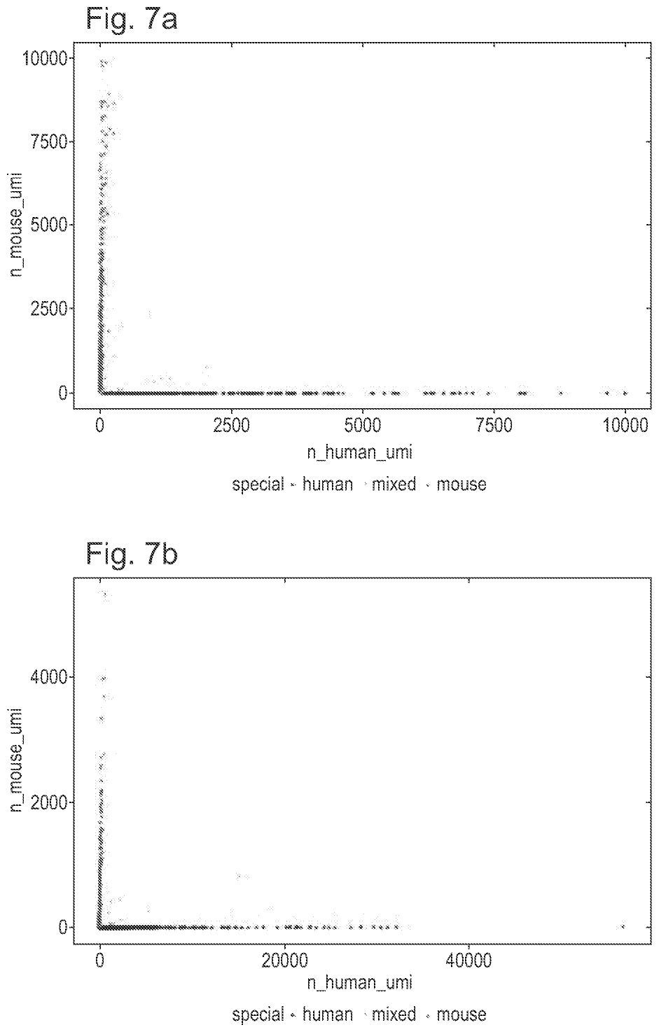

Given that the cell barcode and UMIs are located at the 3′ end of the oligonucleotide, a search was performed for the presence of the polyA or polyT site within 100 nucleotides from the end of the nanopore read. Both a polyA site and the adapter sequence were identified in 62% of reads (FIG. 2e). This initial approach removed a significant proportion of reads with low quality and short reads (FIG. 6). Within these polyA⁺ reads, 55% of the reads with a barcode and UMI sequence were correctly identified (FIG. 2e). 9% of barcodes and UMIs with a perfect nucleotide block complementarity and 60% of reads that had one or more errors within the barcode and UMI sequence were identified. Reads with nucleotide insertions or deletions were removed from the analysis. Next, the barcode correction strategy was applied to the ambiguous reads and 47% of reads were recovered. Application of the UMI deduplication method also led to increased quantification accuracy (FIG. 7).

### Identification of Alternative Transcript Isoform Usage in Myeloma Cell Lines

The majority of genes generate multiple transcripts that give rise to proteins that may carry out distinct and even opposing functions. Identifying the primary transcript associated with a phenotype can assist with the exploration of the underlying molecular mechanisms and with drug development. Short-read droplet based scRNA-seq approaches do not allow isoform usage to be resolved.

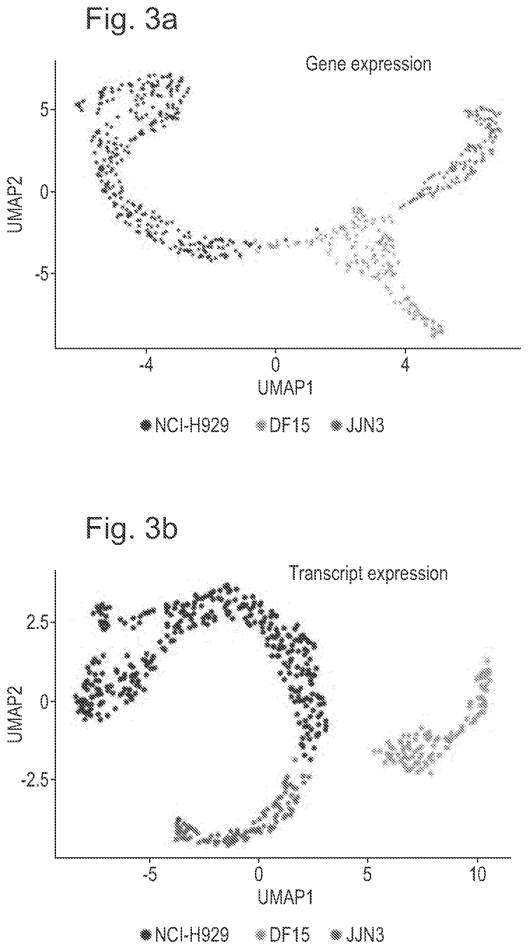

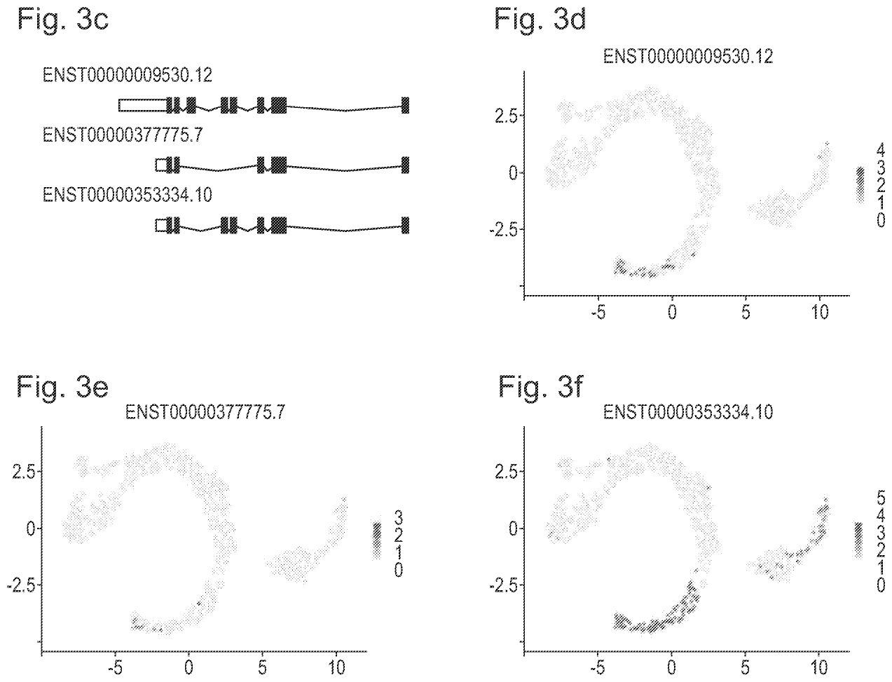

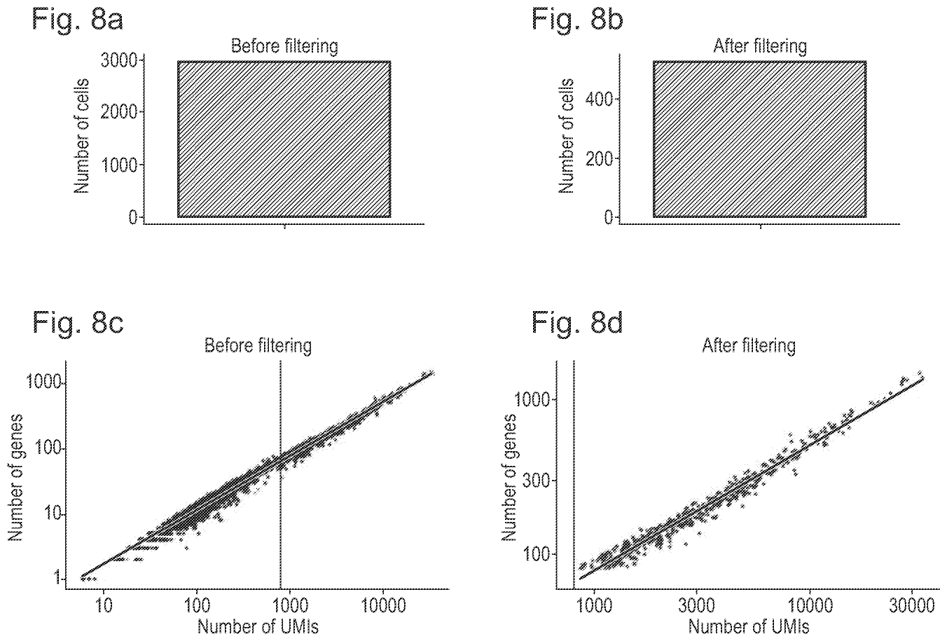

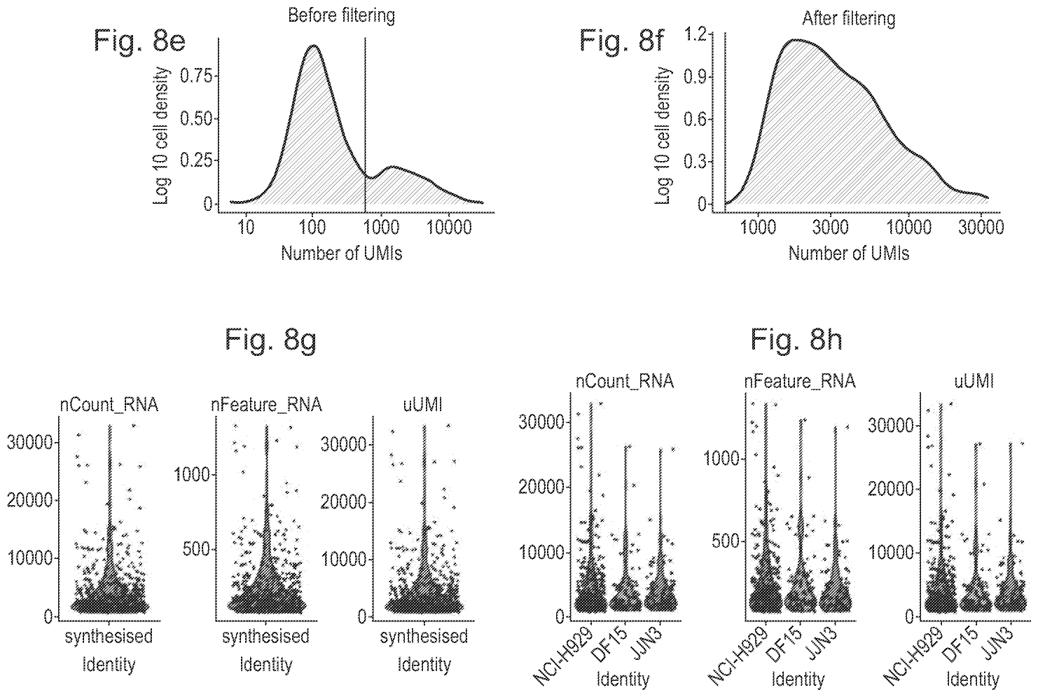

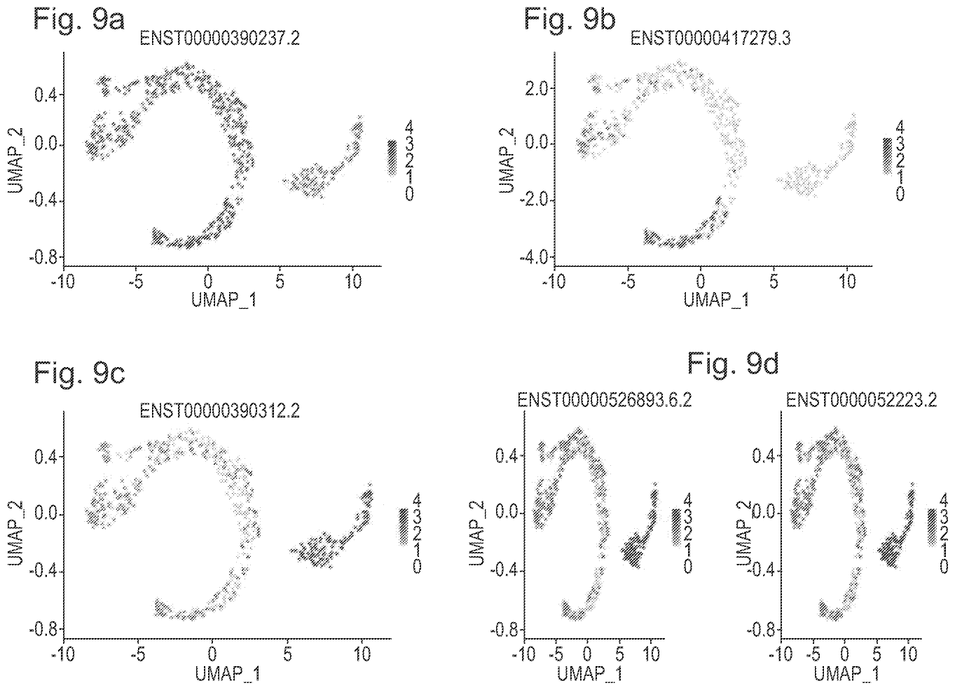

The approach described herein was applied to a mix of NCI-H929, JJN3 and DF15 myeloma cell lines. cDNA synthesis reaction was performed on approximately 500 cells. Following filtering (FIG. 8), nanopore sequencing was capable of sufficiently resolving the different myeloma cell types at both the gene level (FIG. 3a) and the transcript level (FIG. 3b). Interestingly, the clustering is more defined at the transcript level and more diffuse at the gene expression level, potentially reflecting the diversity of transcript usage within these cells. A search was performed for differentially regulated transcripts between cell types and clusters. Cell type specific usage was observed for 359 genes and 416 differentially expressed isoforms. In agreement with the literature and the biology of plasma cells, a significant differential expression for light chain isoform usage we observed (FIG. 9). Interestingly, there was also significant differential isoform usage of CD74 (FIG. 3c, d, e, f), a potential therapeutic target in multiple myeloma (Burton et al. Clin Cancer Res 10 (2004) 6606-11).

### DISCUSSION

Recent advancement in single-cell droplet-based sequencing technologies have enabled molecular profiling of the transcription rates of cells and tissues at the single-cell level. However, transcriptional activity is typically summarised at the gene-level due to the limitations of short-read sequencing technologies that typically only allow 5′ sequencing of a gene. The recent development of long read sequencing technologies such as PacBio or Nanopore sequencing promises to revolutionise the sequencing of full-length transcripts. However, its application to single-cell has been hindered by the high basecalling error rates associated with long read sequencing technologies. This makes it challenging to both accurately assign a read to the correct cell and correct for library preparation PCR duplications.

While others have error corrected single-cell long read sequencing using short read sequencing, the approach described herein provides a per barcode and UMI basecalling accuracy score. By modifying the barcode (and UMI) synthesis methodology, oligonucleotide sequences have been built as described herein using homodimer reverse amidites during the split and pool process. Having a homodimer provides the ability to detect basecalling errors within both the barcode and UMI sequences. Highly accurate barcodes, determined by full complementarity of the blocks of homodimer nucleotides across the full oligonucleotide length, can be used as a guide to error correct barcodes with sequencing errors. Furthermore, directional network approaches, first published by UMI-tools, have been adapted as described herein to account for errors within the UMI sequence. Therefore, single-cell sequencing barcodes and deduplicate UMIs can be error correct with a high level of accuracy.

This approach has multiple advantages over current methodologies to correct error prone sequencing. (i) The approach uses direct nanopore sequencing that negates the need for additional short-read alignment data. (ii) A basecalling accuracy rate can be provided for each barcode and UMI sequenced. (iii) Using the combined accuracy of all barcodes and UMIs, the overall accuracy of single-cell sequencing can approximated, which aids with assessing the quality of the experiment. The barcode and UMI sequencing accuracy information is then applied to recover single-cell barcodes and deduplicate UMI sequencing. We show that this approach can be used to error correct both short read (Illumina) and long read nanopore sequencing data, thereby recovering sequencing data that would otherwise be lost due to barcode miss-assignment.

The present Example shows that synthesising the barcode and UMI sequences using blocks of dimer reverse amidites allows measurement of the error rate within sequencing data. This information can be used to assign barcodes and UMIs that contain errors to the correct cells/samples. Whilst the exemplified method used dimer blocks, the method would work equally well with other pre-defined block sequences, including longer block sequences, as long as each block sequence differs from each other block sequence by at least two nucleotide substitutions.

### Material and Methods

### Cell Lines and Reagents

HEK293T, JJN3, H929 and 3T3 cells were purchased from ATCC. DF15 cells were kindly gifted by Celgene (Now Bristol Myers Squibb). Cell lines were cultured in DMEM low glucose medium supplemented with FBS for no longer than 20 passages. They were mycoplasma tested every 6 months and authenticated during the course of this project.

### Oligonucleotide Synthesis

Bead functionalization and phosphoramidite synthesis was performed by ATDBio. Toyopearl HW-65s resin, purchased from Tosoh Biosciences (product number: 0019815), was used as the solid support for reverse-direction phosphoramidite synthesis. Prior to oligonucleotide synthesis, the initial loading of hydroxyl groups on the resin was reduced via a capping reaction. Capping was performed by suspending the resin in a mixture of acetic anhydride and lutidine in THF, and N-methyl imadazole in acetonitrile for 24 hours. Following capping, the synthesis was performed using an ABI394 DNA synthesiser running a custom 1 micromole synthesis method. The sequence of the capture oligonucleotide is given below:

Bead-5′ TT-HEG-PC-HEG-
TTTTTTTAAGCAGTGGTATCAACGCAGAGTACJJJJJJJJJJJJNNNNNNNNTTTTTTTTTTT
TTTTTTTTTTTTTTTTTTT
N.b. ′J′ indicates a dimer nucleotide added via split and pool synthesis.
′N′ indicates a degenerate dimer nucleotide.

The synthesis method featured an extended coupling time: 5 minutes for the addition of reverse phosphoramidites and 10 minutes for the addition of modified phosphoramidites and dimer phosphoramidites. A total of 60 mg capped resin was used per synthesis; 15 mg per column position.

The barcode was generated via 12 split and pool synthesis cycles. Prior to the first split and-pool synthesis cycle, beads were removed from the synthesis column, suspended in acetonitrile, pooled and mixed, and divided into four aliquots equal in volume. The bead aliquots were then transferred to separate synthesis columns and reacted with either 3′-DMT-dG-dG-5′-CE, 3′-DMT-dC-dC-5′-CE, 3′-DMT-dA-dA-5′-CE, or 3′-DMT-dT-dT-5′-CE phosphoramidite. This process was repeated 11 times. Following the final split and pool cycle, the resin was pooled, mixed and divided between four columns prior to the synthesis of the UMI and poly-T tail. An equimolar mixture of the four dimer phosphoramidites was used in the synthesis of the degenerate UMI region.

Following the synthesis, the resin was washed with DEA (20% in acetonitrile, over a 2 minute period). Subsequently the resin was washed with acetonitrile and dried prior to deprotection in aqueous ammonia (55° C., 6 hours).

Reverse directionality dimer phosphoramidites required for the split and pool and UMI region, were purchased as a custom product from ChemGenes: 3′-DMT-dA(N-Bz)5′-Phosphate-3′-dA(N-Bz)-5′-CE, 3′-DMT-dG(N-iBu)5′-Phosphate-3′-dG(N-iBu)-5′-CE, 3′-DMT-dC(N-Ac)5′-Phosp hate-3′-dC(N-Ac)-5′-CE, 3′-DMT-dT 5′-Phosphate-3′-dT-5′-CE. Reverse directionality monomer phosphoramidites, used for the SMART primer binding site and poly-T tail, were purchased from Linktech: 3′-DMT-dA (N-Bz)-5′-CE, 3′-DMT-dG (N-iBu)-CE-5′, 3′-DMT-dC (N-Ac)-5′-CE, 3′-DMT-dT-5′-CE (Item numbers: 2022, 2021, 2023, 2020). The modified phosphoramidite reagents were purchased from Linktech: Spacer-CE Phosphoramidite (Item number: 2129) and Photocleavable linker-CE (Linktech—item number: 2066).

### Simulated Barcode Data

We simulated barcode sequences with a length of 24 (12 blocks of nucleotides pairs) and then simulated the process of randomly introducing PCR errors and sequencing errors into 95% of our barcodes. We then performed a two-pass barcode assignment strategy in which true barcodes were identified based off the nucleotide pair complementarity across the full length of the barcode. These true barcodes were then used as a guide to correct the remaining barcodes based on approximate string matching (Levenshtein distance). The following values were used as values within our simulations: Sequencing depth 400; number of UMIs 10-100; barcode-length 24; PCR error rate 1×10⁻⁵; sequencing error rate 1×10⁻¹-1×10⁻⁷ and number of PCR cycles 25.

### Simulated UMI Data

We generated simulated UMI data of length 16 (8 blocks of nucleotide pairs) to confirm the accuracy of our UMI correction method. We simulated the process of UMI PCR amplification and sequencing errors seen with nanopore sequencing. UMIs were generated following an approach that was initially proposed by UMI-tools (Smith et al., Genome Res 27 (2017) 491-499). Briefly, each UMI was generated at random, with a uniform probability of amplification (0.8-1.0). We simulated PCR cycles so that each UMI was selected in turn and duplicated according to the probability of amplification. PCR errors were added randomly and then any new UMI sequences were assigned new probabilities of amplification. A defined number of UMIs were randomly sampled to simulate sequencing depth and sequencing errors introduced with a specified probability. Finally, we checked for the presence of mismatched double nucleotides within the UMI and if errors were detected, the UMIs were split into two and then separately collapsed into 8 bp nucleotides. Unambiguous UMIs were collapsed into 8 bp nucleotides without splitting. The number of true UMIs was then estimated from the final pool of UMIs using UMI correction methods proposed in the original UMI-tools manuscript. The following values were used as values within our simulations. Sequencing depth 10-400; number of UMIs 10-100; UMI-length 6-16; PCR error rate 1×10⁻3-1×10⁻⁵; sequencing error rate 1×10⁻¹-1×10⁻⁷ and number of PCR cycles 4-12.

Droplet Based scRNA-Seq

Single-cell capture and reverse transcription (RT) were performed using the Drop-seq approach, as previously described (Macosko et al., Cell 161 (2015) 1202-1214). Briefly, cells were loaded into the DolomiteBio Nadia system microfluidic cartridge at a concentration of 310 cells per microliter. Oligonucleotide beads were synthesised by ATDBio (Oxford, UK). Beads were loaded into the microfluidic cartridge. Cell capture and lysis were performed according to the Nadia instrument manufacturer's instructions (DolomiteBio). The droplet emulsion was then disrupted using lml of 1H, 1H, 2H, 2H-Perfluoro-1-octanol (PFO; Sigma) and beads released into aqueous solution. Following several washes, the beads were then subjected to RT. Prior to PCR amplification, the beads were washed and then treated with ExoI for 45 mins. PCR was then performed using the SMART PCR primer (AAGCAGTGGTATCAACGCAGAGT) and then cDNA purified using AMPure beads (Beckman Coulter). In order to achieve a high concentration of cDNA the input was subjected to 25 cycles of PCR amplification, rather than the 13 stated in the original Drop-seq protocol. Finally, cDNA was quantified using a TapeStation (Agilent Technologies) DNA high sensitivity d5000 tape before being split for Illumina or Nanopore library generation.

### Single-Cell Illumina Library Preparation for Sequencing

Library prep for illumine was performed as previously described (Macosko et al., Cell 161 (2015) 1202-1214). Briefly, purified cDNA was used as an input for the Nextera XT DNA library preparation kit (Illumina). Library quality and size was determined using a TapeStation (Agilent Technologies) High Sensitivity d1000 tape. High quality samples were then sequenced to a minimum of 50,000 reads per cell on a NextSeq 500 sequencer (Illumina) using a 75-cycle High Output kit using a custom read1 primer

(GCCTGTCCGCGGAAGCAGTGGTATCAACGCAGAGTAC).

### Nanopore Library Preparation for Sequencing

Full length cDNA samples were prepared using Oxford Nanopore Technologies SQK-LSK-109 Ligation Sequencing Kit, with the following modifications. Incubation times for end-preparation and A-tailing were lengthened to 15 minutes and all washes were performed with 1.8× AMPure beads to improve recovery of smaller fragments. SFB was used for the final wash of libraries. 50 fmol of library were sequenced on MinION FLO-MIN106D R9.4.1 flow cells according to the manufacturers protocol.

Illumina-Based scRNA-Seq Analysis Workflow

The fastq data was processed using a custom written cgatcore pipeline (https://github.com/Acribbs/aattggcc). We identified ambiguous and unambiguous reads based on the occurrence of dual nucleotide complementarity within the barcode sequence. The unambiguous barcodes were then used to error correct the ambiguous reads by fuzzy searching using a Levenshtein distance of 4 (unless stated in the figure legend). The barcode and UMI sequence for the corrected read pairs were then collapsed into single nucleotide sequences. The resulting fastq files were used as an input for Kallisto (v0.46.1) bustools (v0.39.3) (Melsted et al., bioRxiv (2019) 673285), which was used to generate a counts matrix. This counts matrix was used as an input to the standard Seurat pipeline (v3.1.4) (Stuart et al., Cell 177 (2019) 1888-1902 e21).

Nanopore-Based scRNA-Seq Analysis Workflow

We performed basecalling on the raw fast5 data using Guppy (v) (guppy_basecaller-compress-fastq-c dna_r9.4.1_450bps_hac.cfg-x “cuda:1”) in GPU mode from Oxford Nanopore Technologies running on a GTX 1080 Ti graphics card. For each read we identify the barcode and UMI sequence by searching for the polyA region and flanking regions before and after the barcode/UMI. Accurately sequenced barcodes were identified based on their dual nucleotide complementarity. Unambiguous barcodes were then used as a guide to error correct the ambiguous barcodes in a second pass correction analysis approach. We performed fuzzy searching using a Levenshtein distance of 4 (unless otherwise stated in the figure legend) and replaced the original ambiguous barcode with the unambiguous sequence. A whitelist of barcodes was then generated using UMI-tools whitelist (umi_tools whitelist--bc-pattern=CCCCCCCCCCCCCCCCCCCCCCCCNNNNNNNNNNNNNNNN--set-cell-number=1000)(Smith et al., Genome Res 27 (2017) 491-499). This whitelist was used to assess the quality of our cells to read count ratio and used as an input for UMI-tools extract. Next the barcode and UMI sequence of each read was extracted and placed within the read2 header file using UMI-tools extract (umi_tools extract--bc-pattern=CCCCCCCCCCCCCCCCCCCCCCCCNNNNNNNNNNNNNNNN--whitelist=whitelist.txt). Reads were then aligned to the transcriptome using minimap2 (Li, Bioinformatics 34 (2018) 3094-3100) (-ax splice-uf--MD--sam-hit-only--junc-bed) using the reference transcriptome for human hg38 and mouse mm10. The resulting sam file was converted to a bam file and then sorted and indexed using samtools (Li et al., Bioinformatics 25 (2009) 2078-9). The transcript name was then added as a XT tag within the bam file using pysam. Finally, UMI-tools count (umi_tools count-per-gene-gene-tagXT-per-cell-double-barcode) was used to count features to cells before being converted to a market matrix format. We modified UMI-tools count to handle the double nucleotide UMIs as defined below. This counts matrix was then used as an input into the standard Seurat pipeline.

### UMI Error Correction

UMI-tools was forked on Github and the counts functionality was (https://github.com/Acribbs/UMI-tools) modified to handle our double oligonucleotide design. Briefly, ambiguous UMIs were split into two and then separately collapsed into 8 bp nucleotides. Unambiguous UMIs were collapsed into 8 bp nucleotides without splitting. The directional method implemented within the original UMI-tools was then performed to correct UMI sequencing errors.

### Dimensionality Reduction and Clustering

Raw transcript expression matrices generated by UMI-tools count (for nanopore data) or kallisto bustools (for Illumina data) were processed using R/Bioconductor (v4.0.3) and the Seurat package (v3.1.4). Gene matrices were cell level scaled and log transformed. The top 2000 highly variable genes were then selected based on variance stabilising transformation which was used for principal component analysis (PCA). Clustering was performed within Seurat using the Louvain algorithm. To visualise the single-cell data, we projected our data onto a Uniform Manifold Approximation and Projection (UMAP).

### Differential Gene Expression

Differential expression analysis was performed using nonparametric Wilcoxon test on log₂(TPM) expression values. Differentially expressed transcripts were selected based on the basis of absolute log 2 fold change of >1 and the adjusted P value of <.0.05.

### Data Availability

All relevant sequencing data has been deposited to GEO under the accession number: GSE162053.

### Code Availability

Source data is provided with this manuscript. All custom pipelines used within the analysis is available on Github (https://github.com/Acribbs/aattggcc). Modifications to the UMI-tools code is also available as a fork on Github (https://github.com/Acribbs/UMI-tools).

### Reference Example 1—Exemplary Protocol for 3′ End mRNA Capture and Library Generation from Single Cells

### Reagents and Buffers:

Reagent
Cell buffer
PBS
BSA
Template switch oligo (TSO)
Lysis buffer
RT buffer (5X)
Ficoll PM-400
Triton X-114
Maxima H-Rev transcriptase
ATP 100 mM
dNTPs
RNAse Inhibitor
QX200± ™ Droplet Generation Oil for EvaGreen
Perflurooctanol (PFO)
PCR mix
Kapa HiFi polymerase
PCR primer
AMPure beads
BioAnalyzer High Sensitivity D5000 tape
BioAnalyzer High Sensitivity D1000 tape
Nextera XT DNA Library Preperation kit
70 μm Flowmi filter
Neubauer Improved Haemocytometer
Fuchs-Rosenthal Haemocytometer (plastic)

### Reagents and Buffers:

Lysis buffer is preferably made fresh. Cell buffer is prepared fresh and filtered through a 0.2 μm syringe filter.

| Concentration/volume | Component |
|---|---|
| Lysis buffer | |

| | | |
|---|---|---|
| 100 | μL | 5X RT buffer |
| 80 | μL | Ficoll PM-400 |
| 1.25 | μL | Triton-X114 |
| 20 | μL | dNTP |
| 20 | μL | RT enzyme |
| 5 | μL | RNAse Inhibitor |
| 2 | μL | ATP |
| 21.75 | μL | H₂O |
| 250 | μL | |

| | |
|---|---|
| Cell buffer | |
| 1X | PBS |
| 0.01% | BSA |

| | | |
|---|---|---|
| 15 | μL | TSO |
| 10 | μL | UDG |
| 10 | μL | APE-1 |

### Primers

| Name | Sequence |
|---|---|
| Template switch oligo | 5′-AAGCAGTGGTATCAACGCAGAGTGAATrGrGrG |
| SMART PCR PRIMER | 5′-AAGCAGTGGTATCAACGCAGAGT |
| New-P5-SMART PCR hybrid oligo | 5′-AATGATACGGCGACCACCGAGATCTACACGCCT |
| | GTCCGCGGAAGCAGTGGTATCAACGCAGAGT* |
| | A*C |
| Read1 Custom primer | 5′- |
| | GCCTGTCCGCGGAAGCAGTGGTATCAACGCAG |
| | AGTAC |

Capture beads:

5′-TCGGACCGTTCGTCGGTGGTATCAACGCAGAGTACJJJJJJJJJJJJNNNNNNGTCCGAGCGTAGGTTATCCGTTTTT
TTTTTTTTTTTTTTTTTTTTTTTTTUCCGCG-HEG-CGCGGAAAAAA-HEG-PCLinker-T-Bead-3′

rG=ribonucleic acid Guanine
*=Phosphorothioated DNA nucleotides
HEG=Spacer-CE Phosphoramidite 18 (aka spacer 18)
N=unique molecular identifier (UMI) nucleotide
J=barcode (BC) sequence nucleotide
PCLinker=PC Linker-CE Phosphoramidite (3-(4,4′-Dimethoxytrityl)-1-(2-nitrophenyl)-propane-1,3-diol-[2-cyanoethyl-(N,N-diisopropyl)]-phosphoramidite)
   Beads in dry resin form require washing with 20 mL of 100% ethanol and then washing with 30 ml of TE-TW and resuspension in 20 mL of TE-TW. Bead numbers are determined using a Fuchs-Rosenthal Haemocytometer (plastic). Beads are stored at +4° C. for 6 months or longer.

### Encapsulation

Power up the Nadia instrument with the desired chip and place it on the instrument
Ensure the locating pins on the instrument slot into corresponding cut-outs in the cartridge
Remove the gasket from the chip.
Follow on-screen instructions and using a P1000 pipette or a powered aspirator/dispenser, load 3 ml of emulsion oil as required into the oil reservoir
Re-apply the gasket making sure the gasket fits over the 4 pins in the corners.
Press Next to begin pre-cooling
   Bead preparation:
Preferably use low-retention pipette tips to minimise bead loss.
Prepare 1 ml of lysis buffer and place on ice
Transfer 155,000 beads from the stock suspension into a new tube and spin down at 1,000 g for 1 min, discard the supernatant using a P200 pipette leaving only the remaining pelleted beads
Resuspend the beads in 250 μl of cold Lysis buffer and store on ice until needed

### Cell Preparation:

Prepare the cell buffer according to the buffer instructions. The TSO is added to the cell buffer as it is required following in droplet reverse transcription (RT).

Prepare the cells in a single cell suspension as desired. The protocol below uses HEK or 3T3 cells.
Trypsinise cells for 5 mins using TrypLE
Spin down cells at 300 g for 5 mins
Resuspend the cells in lml of 1×PBS
Spin down cells at 300 g for 3 mins
Remove supernatant and resuspend in Cell buffer (minus TSO)
Sieve the cell through a 70 μm (you can also use 40 μm if required) and count in a cell counter or haemocytometer.
Spin down cells and resuspend in cold Cell Buffer (310 cells/μl). If mixed species then take 38,250 cells from each for the total 77,500 cells and pool into a final volume of 250 μl. Alternatively 77,500 single cells of the same cell type can be used. Place cells on ice until needed.

### Run Cells on the Nadia Instrument:

Press ‘Next’ on the Nadia screen to open the lid
Follow on-screen instructions to load the beads into the blue flashing wells:
   Carefully mix the beads about 10× by using standard P200 pipette tips
   Set the pipette to 125 μl and load 250 μl of bead suspension using low retention tips
Next, cells are loaded into the orange flashing wells:
   Carefully resuspend the cells using a P200
   Set the pipette to 125 μl and load 250 μl of cells.
Replace the gasket and close the lid
Start the run. The Nadia instrument will take around 25 mins to finish for a cell run and 1.5 hours for a single-nuclei run.
When the run is complete an emulsion will be present in the output well of the chip. The emulsion should be creamy white in appearance and will be floating atop a layer of oil.
Using a P1000 pipette tip remove as much oil from the well as possible but be careful not to remove beads.
With some residual oil in the well remaining, pipette the oil and emulsion into a 1.5 ml low bind Eppendorf (from now on use exclusively low bind tubes).

### UV Irradiate the Beads:

Irradiate the beads using UV light so that the photocleavable linker breaks the connection between the bead and the oligonucleotide. The oligo is then free in solution and can bind RNA.
Open the cap of the 1.5 ml Eppendorf and place UVP dual tube handheld UV lamp above the tubes
Turn on the UV lamp and set it to long wave setting
UV irradiation is complete after 10 min.

### Create Free 3′ OH:

Incubate the 1.5 ml Eppendorf at 37° C. for 1 hr

### Reverse Transcription:

Incubate the tube at room temperature (22° C.) for 30 mins
Incubate the tube at 42° C. for 1.5 hours

### Breaking Emulsion and Clean-Up:

Add 100 μl of PFO to the sample then invert tubes three times
Spin down sample at 1000 g for 1 min
Collect the aqueous phase (about 250 μl) and add to a new 1.5 ml tube.
Spin down at 1000 g for 1 min again and remove sample to new 1.5 ml tube to remove residual beads
   Purification of cDNA:
Vortex bottle of AMPPure beads to mix
Add 1× (1:1 beads to sample ratio, usually 250 μl) of AmpPure XP beads to each tube of sample.
Perform the clean-up following manufacturers protocol
Elute in 25 μl of H₂O
   PCR with Kapa HiFi:

Following addition of the SMART adapters following RT (using TSO) in the previous step, perform PCR amplification of the cDNA using primers against the SMART sequence.
Defrost master mix and SMART PCR primer
Place 24.6 μl of cDNA into a PCR tube and then add the following components:

| Volume (μl) | Component |
|---|---|
| 24.6 | cDNA |
| 0.4 | 100 μM SMART PCR primer |
| 25 | 2X Kapa HiFi MM |

Then proceed to PCR:
If running a nanopore experiment then increase the total cycles of PCR to 25:

| Cycles | T [° C.] | Time | |
|---|---|---|---|
| | 95 | 3 | min |
| 4X | 98 | 20 | s |
| | 65 | 45 | s |
| | 72 | 3 | min |
| 9X | 98 | 20 | s |
| | 67 | 20 | s |
| | 72 | 3 | min |
| | 72 | 5 | mins |

| | |
|---|---|
| 4 | ∞ |

Purification of cDNA and Evaluate on Tapestation:
Vortex bottle of AMPPure beads to mix
Add 0.6× (0.6:1 beads to sample ratio, usually 250 μl) of AmpPure XP beads to each tube of sample.
Perform the clean-up following manufacturers protocol
Flute in 20 μl of H₂O
Run 2 μl of sample on a d5000 high sensitivity tape and run on Tapestation according to manufacturer's instructions.
The cDNA may produce a spiky or smooth but even trace with an average size of 1300-2000 bp.

### Next an Illumina or Nanopore Library can be Generated

### Nanopore Library

A minimum of 200 ng of cDNA is used as an input to the SQK-LSK109 or SQK-LSK110 library prep kit from Oxford nanopore. The library is prepared following manufacturer's instructions.

### Illumina Library

Tagmentation of cDNA and Library Prep with Nextera XT:

This step will tagment the DNA and add indexes to generate a sequencing library.
Preheat a thermocycler to 55° C.
For each sample, combine a minimum of 600 pg (ideally 1800 pg is) to a total volume of 5 μl.
Add 10 μl of Tagment DNA buffer (RD) and 5 μl of Amplicon Tagment Mix (ATM) bringing the total volume to 20μ.
Mix by pipetting up and down 5 times and spin to ensure liquid is at the bottom of the tube.
Incubate at 55° C. for 5 mins
Add 5 μl of Neutralization Tagment buffer (NT) and mix by pipetting up and down 5 times and spin down
Incubate at room temperature for 5 mins
Add into each PCR tube the following components in the order specified:

| Volume [μl]] | Component |
|---|---|
| 15 | Nextera PCR MM (NPM), Kapa HiFi also works as a |
| | replacement |
| 8 | H₂O |
| 1 | 10 μM New-P5-SMART PCR oligo |
| 1 | 10 μM Nextera N70X oligo |

Run PCR program:

| Cycles | T [° C.] | Time | |
|---|---|---|---|
| | 95 | 30 | s |
| 12 | 95 | 10 | s |
| | 55 | 30 | s |
| | 72 | 30 | s |
| | 72 | 5 | min |

| | |
|---|---|
| 4 | ∞ |

Purification of cDNA and Evaluate on Tapestation:
Vortex bottle of AMPPure beads to mix
Add 50 μl of H₂O to sample to make a final volume of 100 μl
Add 60 μl (0.6:1 beads to sample ratio, usually 250 μl) of AmpPure XP beads to each tube of sample.
Incubate 5 mins at room temperature
Place sample in magnet at high position
Transfer 150 μl of supernatant into a new tube
Add 20 μl of beads to the sample and mic up and down 5 times
Incubate for 5 mins at room temperature
Place in the magnet at high position
Remove supernatant and discard
Add 200 μl of 85% ethanol to wash the beads, wait 30 s
Remove the ethanol
Repeat ethanol wash twice
Centrifuge briefly and then return to magnet on low position
Remove residual ethanol and then wait for 2 mins
Elute in 20 μl of H₂O
Store at 4° C. for 72 hours or at −20° C. for long term storage

Run 2 μl of sample on a d1000 tapestation. Tagmented libraries should have a fairly smooth trace with an average size of 500-680 bp. You now have a library for sequencing which can be performed following illumine protocols.

### Reference Example 2

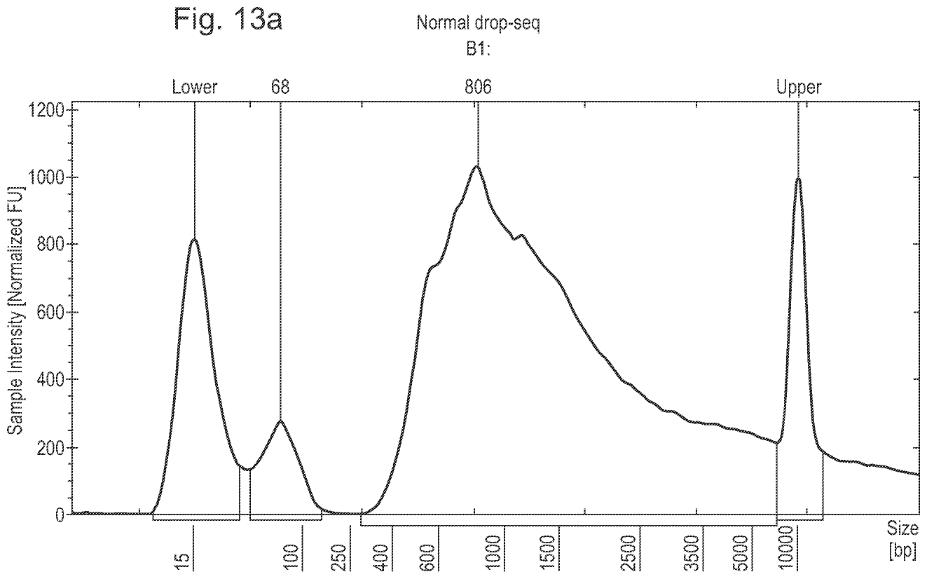

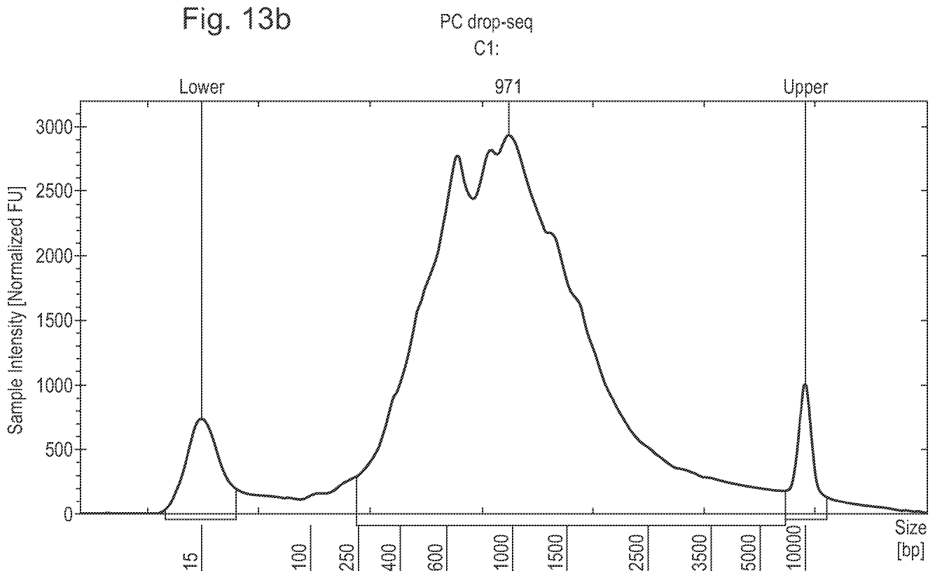

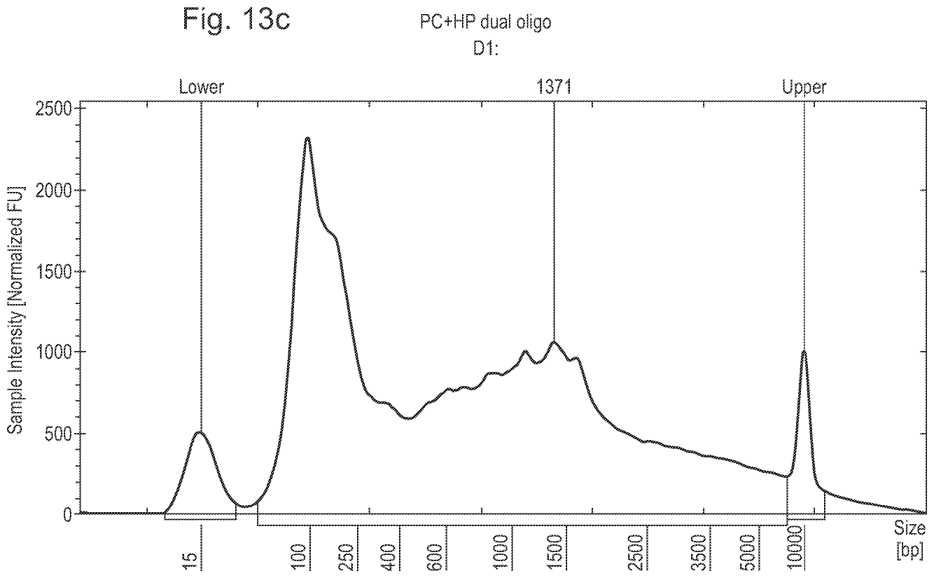

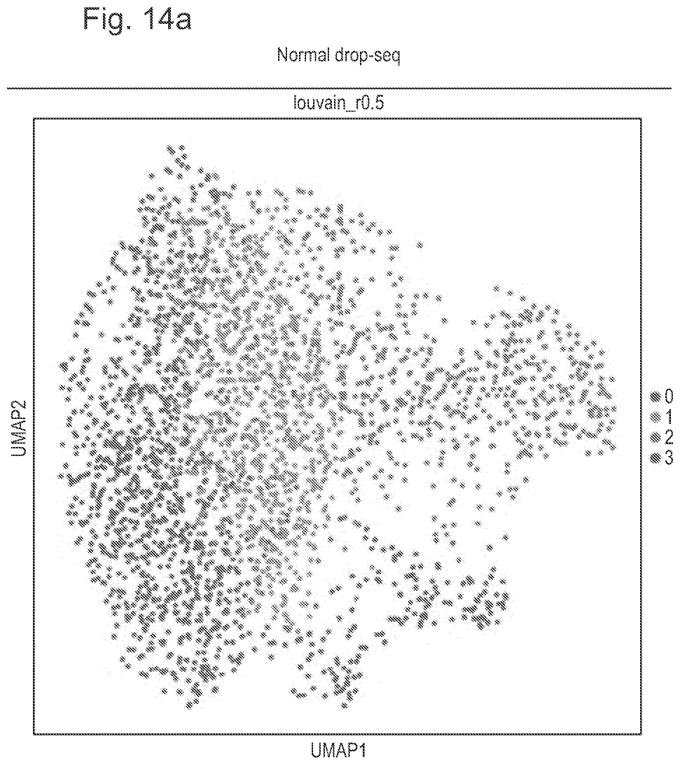

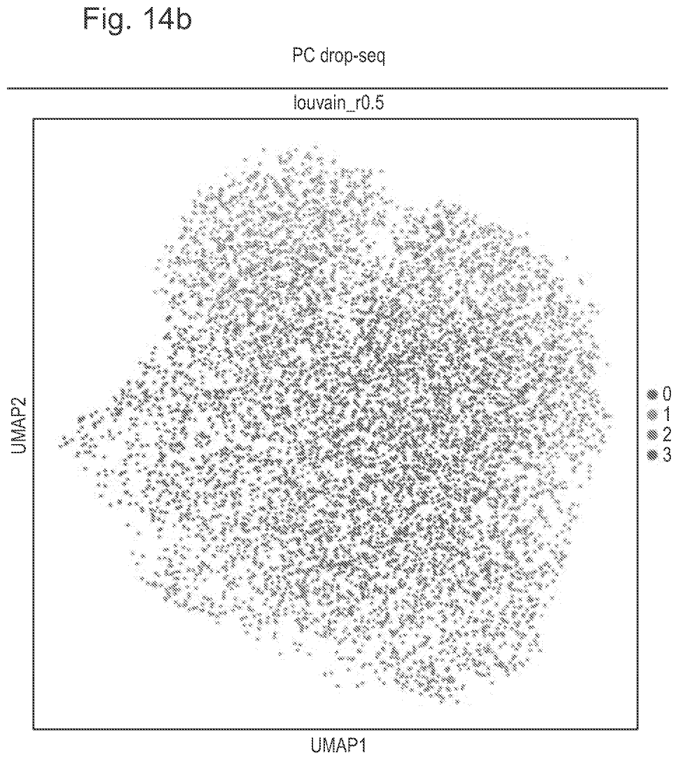

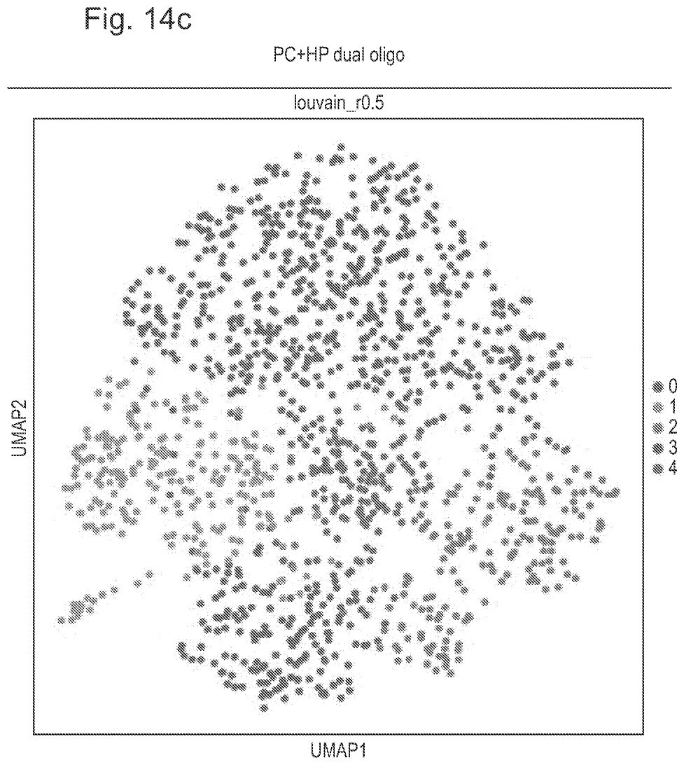

HEK293T cells were harvested and encapsulated as described in Reference Example 1. Tapestation traces show the final library produced using three different protocols: A. Normal drop-seq using the bead sequence from the published EZ Macosko-2015 method for performing droplet based sequencing (FIG. 13A); B. PC drop-seq—This same sequence from EZ Macosko 2015, but with a photocleavable linker added at the 5′ end of the sequence; and C. PC+HP dual oligo-tapestation trace shows RNA library generated using the oligonucleotide sequence described in Reference Example 1. The libraries were sequenced using Illumina Next seq 500 machine. UMAP plots show the number of cells measured using each of the three methods (FIG. 14, A-C). The results demonstrate that the bead oligonucleotides described in Reference Example 1 are able to capture RNA that can be amplified to produce sequencing libraries of cellular RNA.

### Reference Example 3—Exemplary Protocol for 3′ End mRNA Capture and 5′ End DNA Capture

The protocol is the same as set out in Example 1, except as follows:

Lysis buffer additionally includes 10 μL of KlenTaq and 10 μL T7 ligase. Cell buffer additionally includes 10 uL of blocking oligo.

### Primers:

| Name | Sequence |
|---|---|
| Template switch oligo | 5′-AAGCAGTGGTATCAACGCAGAGTGAATrGrGrG |
| SMART PCR PRIMER | 5′-AAGCAGTGGTATCAACGCAGAGT |
| New-P5-SMART PCR hybrid oligo | 5′-AATGATACGGCGACCACCGAGATCTACACGCCT |
| | GTCCGCGGAAGCAGTGGTATCAACGCAGAGT* |
| | A*C |
| Read1 Custom primer | 5′-GCCTGTCCGCGGAAGCAGTGGTATCAACGCAG |
| | AGTAC |
| Captured DNA forward | 5′-TCGTCGGCAGCGTCAGATGTGTAT |
| Captured DNA reverse | 5′-CGGATAACCTACGCTCGGAC |
| DNA final i7 | 5′-CAAGCAGAAGACGGCATACGAGATTCGCCT |
| | TAGTCTCGTGGGCTCGGAGATGTGTATAAGAG |
| | ACAGCGGATAACCTACGCTCGGAC |
| ATAC i5 | 5′-AATGATACGGCGACCAC |
| | CGAGATCTACACTCGTCGGCAGCGTCAGATGTG |
| MEDSB Blocking oligo | 5′-P-GACGCTGCCGACG-InvA |

Inv=Inverted base

### Capture Beads:

5′-TCGGACCGTTCGTCGGTGGTATCAACGCAGAGTACJJJJJJJJJJJJNNNNNNGTCCGAGCGTAGGTTATCCGTTTTT
TTTTTTTTTTTTTTTTTTTTTTTTTUCCGCG-HEG-CGCGGAAAAAA-HEG-PCLinker-T-Bead-3′

Heat to Remove Tn5 from the DNA:

This step is carried out in between incubation at 37 C to create the 3′ hydroxyl groups and the reverse transcription step, and removes the bound Tn5 from the DNA so the oligo can capture ATAC'd DNA.
Incubate the 1.5 ml tube at 72° C. for 5 mins
   PCR of cDNA Using Kapa HiFi:

Following addition of the SMART adapters following RT (using TSO) PCR amplification of the cDNA is performed using primers against the SMART sequence, as in the RNA-only protocol of Example 1. However, an additional initial PCR of 6 cycles is performed so that both ends of the oligo are amplified. Then the reactions are cleaned up and split between two PCR reactions, one to amplify the DNA captured end and the other to further amplify the RNA captured end.
Defrost master mix and SMART PCR primer
Place 24.6 μl of cDNA into a PCR tube and then add the following components:

| Volume (μl) | Component |
|---|---|
| 24.6 | CDNA |
| 0.4 | 100 μM Captured DNA F |
| 0.4 | 100 μM Captured DNA R |
| 0.4 | 100 μM SMART PCR primer |
| 25 | 2X Kapa HiFi MM |

Then proceed to PCR:

| Cycles | T [° C.] | Time | |
|---|---|---|---|
| | 95 | 3 | min |
| 4X | 98 | 20 | s |
| | 65 | 45 | s |
| | 72 | 3 | min |
| 1X | 98 | 20 | s |
| | 67 | 20 | s |
| | 72 | 3 | min |
| | 72 | 5 | mins |

| | |
|---|---|
| 4 | ∞ |

Purification of cDNA and Evaluate on Tapestation:
Vortex bottle of AMPPure beads to mix
Add 0.6× (0.6:1 beads to sample ratio, usually 250 μl) of AmpPure NIP beads to each tube of sample.
Perform the clean-up following manufacturers protocol
Elute in 2 of H₂O

### PCR Amplify Captured DNA:

This PCR will amplify the captured DNA end of the oligo and add the i7 and i5 adapters, which can then be directly sequenced on an illumina machine.
In a PCR add the following components:

| Volume (μl) | Component |
|---|---|
| 2.5 | 25 μM DNA final PCR primer |
| 2.5 | 25 μM ATAC i5 |
| 5 | DNA product from “PCR of cDNA using |
| | kapa HiFi” |
| 25 | 2X Kapa HiFi MM |

Then proceed to PCR:

| Cycles | T [° C.] | Time | |
|---|---|---|---|
| | 95 | 3 | min |
| 4X | 98 | 20 | s |
| | 65 | 45 | s |
| | 72 | 3 | min |
| 5X | 98 | 20 | s |
| | 67 | 20 | s |
| | 72 | 3 | min |
| | 72 | 5 | mins |

| | |
|---|---|
| 4 | ∞ |

### PCR Amplify Captured RNA End of the Oligo:

Add the following components to a PCR tube:

| Volume (μl) | Component |
|---|---|
| 15 | DNA product from “PCR of cDNA using |
| | kapa HiFi” |
| 4.6 | H₂O |
| 0.4 | 100 μM SMART PCR primer |
| 25 | 2X Kapa HiFi MM |

Then perform PCA as follows:

| Cycles | T [° C.] | Time | |
|---|---|---|---|
| | 95 | 3 | min |
| 4X | 98 | 20 | s |
| | 65 | 45 | s |
| | 72 | 3 | min |
| 9X | 98 | 20 | s |
| | 67 | 20 | s |
| | 72 | 3 | min |
| | 72 | 5 | mins |

| | |
|---|---|
| 4 | ∞ |

### Complete Protocol as in Example 1.

### Reference Example 4

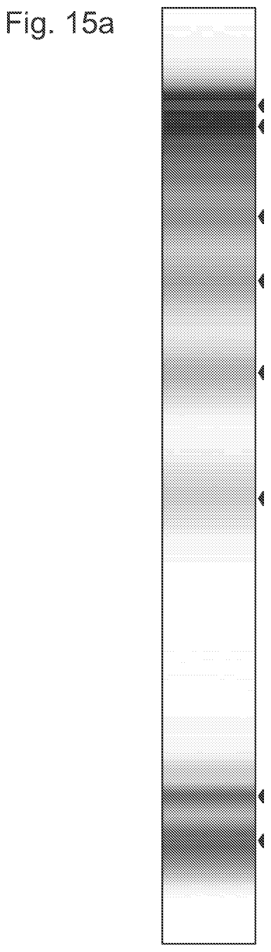

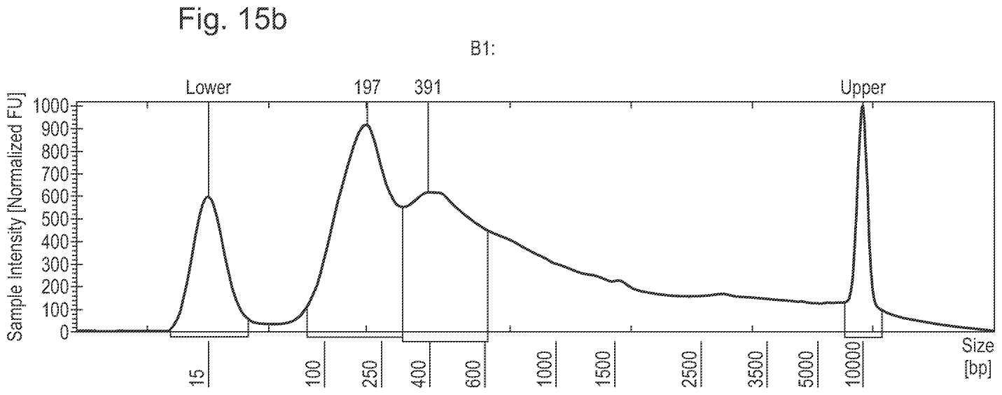

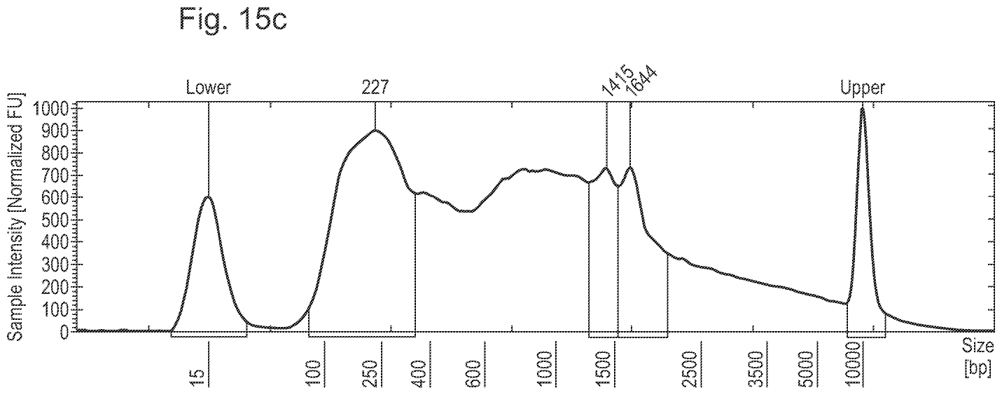

HEK293T cells were harvested and ATAC was performed then encapsulated as described in Example 3. Tapestation trace (FIG. 15) shows both a DNA library produced from the 5′ capture and an RNA library produced from the 3′ capture, the final library generated by amplifying the DNA hybridized at the 5′ end of the dual oligonucleotide. The results demonstrate that the bead oligonucleotides described in Example 3 are able to capture both RNA and DNA that can be amplified to produce sequencing libraries.

Full length SARS-COV-2 structural protein sequences
Surface glycoprotein alias Spike 1273 aa NCBI Reference Sequence: YP_009724390.1
MFVFLVLLPLVSSQCVNLTTRTQLPPAYTNSFTRGVYYPDKVFRSSVLHSTQDLFLPFFSNVTWFHAIHV
SGTNGTKRFDNPVLPFNDGVYFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLREFVFKNIDGYFKIYSKHTPI
NLVRDLPQGFSALEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPCNGVEGENCYF
PLQSYGFQPTNGVGYQPYRVVVLSFELLHAPATVCGPKKSTNLVKNKCVNFNFNGLTGTGVLTESNKKEL
PFQQFGRDIADTTDAVRDPQTLEILDITPCSFGGVSVITPGTNTSNQVAVLYQDVNCTEVPVAIHADQLT
PTWRVYSTGSNVFQTRAGCLIGAEHVNNSYECDIPIGAGICASYQTQTNSPRRARSVASQSIIAYTMSLG
AENSVAYSNNSIAIPTNFTISVTTEILPVSMTKTSVDCTMYICGDSTECSNLLLQYGSFCTQLNRALTGI
AVEQDKNTQEVFAQVKQIYKTPPIKDFGGFNFSQILPDPSKPSKRSFIEDLLENKVTLADAGFIKQYGDC
LGDIAARDLICAQKFNGLTVLPPLLTDEMIAQYTSALLAGTITSGWTFGAGAALQIPFAMQMAYRENGIG
VTQNVLYENQKLIANQFNSAIGKIQDSLSSTASALGKLQDVVNQNAQALNTLVKQLSSNFGAISSVLNDI
LSRLDKVEAEVQIDRLITGRLQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGTHWFVTQRNFYEPQIITTDNT
FVSGNCDVVIGIVNNTVYDPLQPELDSFKEELDKYFKNHTSPDVDLGDISGINASVVNIQKEIDRLNEVA
KNLNESLIDLQELGKYEQYIKWPWYIWLGFIAGLIAIVMVTIMLCCMTSCCSCLKGCCSCGSCCKFDEDD
SEPVLKGVKLHYT
Nucleocapsid phosphoprotein 419 aa NCBI Reference Sequence: YP_009724397.2
MSDNGPQNQRNAPRITFGGPSDSTGSNQNGERSGARSKQRRPQGLPNNTASWFTALTQHGKEDLKFPRGQ
GVPINTNSSPDDQIGYYRRATRRIRGGDGKMKDLSPRWYFYYLGTGPEAGLPYGANKDGIIWVATEGALN
TPKDHIGTRNPANNAAIVLQLPQGTTLPKGFYAEGSRGGSQASSRSSSRSRNSSRNSTPGSSRGTSPARM
AGNGGDAALALLLLDRLNQLESKMSGKGQQQQGQTVTKKSAAEASKKPRQKRTATKAYNVTQAFGRRGPE
QTQGNFGDQELIRQGTDYKHWPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADLDDFSKQLQQSMSSADSTQA
Envelope protein 75 aa NCBI Reference Sequence: YP_009724392.1
MYSFVSEETGTLIVNSVLLFLAFVVFLLVTLAILTALRLCAYCCNIVNVSLVKPSFYVYSRVKNLNSSRV
PDLLV
Membrane glycoprotein 222 aa NCBI Reference Sequence: YP_009724393.1
MADSNGTITVEELKKLLEQWNLVIGFLFLTWICLLQFAYANRNRFLYIIKLIFLWLLWPVTLACFVLAAV
YRINWITGGIAIAMACLVGLMWLSYFIASFRLFARTRSMWSFNPETNILLNVPLHGTILTRPLLESELVI
GAVILRGHLRIAGHHLGRCDIKDLPKEITVATSRTLSYYKLGASQRVAGDSGFAAYSRYRIGNYKLNTDH
SSSSDNIALLVQ

1. A method of adding at least one identifier sequence to each of an array of polynucleotides during synthesis, wherein the identifier sequence comprises a plurality of nucleotide blocks, wherein the method comprises adding each nucleotide block of the identifier sequence by elongating the polynucleotides using a pool of pre-synthesised nucleotide blocks for incorporation into the polynucleotides, wherein the pool of pre-synthesised nucleotide blocks used to add each nucleotide block of the identifier sequence comprises a mixture of different nucleotide block sequences, wherein each nucleotide block sequence in the pool used to add each nucleotide block of the identifier sequence differs from each other nucleotide block sequence in the pool by at least two nucleotide substitutions, and wherein the method adds a different identifier sequence to each of at least 100 different polynucleotides of the array.

2. The method of claim 1, wherein the pre-synthesised nucleotide blocks comprise nucleotide phosphoramidites.

3. The method according to claim 1 or claim 2, wherein the at least one identifier sequence is added to the polynucleotides using degenerate polynucleotide synthesis using the mixed pool of pre-synthesised nucleotide blocks.

4. The method according to any one of the preceding claims, wherein the at least one identifier sequence is added using split-and-pool polynucleotide synthesis comprising
(vii) splitting the array of polynucleotides or the sub-arrays into groups;
(viii) combining each group with a different sub-pool of the pre-synthesised nucleotide blocks, wherein the pre-synthesised nucleotide blocks of each sub-pool have different nucleotide block sequences;
(ix) polynucleotide synthesis to add one pre-synthesised nucleotide block from the sub-pools to the polynucleotides of each respective group;
(x) isolating the polynucleotides of the array from the sub-pools of pre-synthesised nucleotide blocks;
(xi) optionally mixing the polynucleotides from the groups together; and
(xii) repeating steps (i) to (v), allocating different combinations of the polynucleotides or sub-arrays to different groups compared to the previous round.

5. An array of polynucleotides, wherein each polynucleotide of the array comprises an identifier sequence, wherein the identifier sequence of each polynucleotide consists of a consecutive series of at least three nucleotide blocks, wherein each nucleotide block of the identifier sequences is selected from a pool of up to 36 nucleotide block sequences, wherein each nucleotide block sequence of the pool differs from each other nucleotide block sequence of the pool by at least two nucleotide substitutions, and wherein the array comprises at least 100 polynucleotides each having a different identifier sequence.

6. The method according to any one of claims 1 to 4, or the array of polynucleotides according to claim 5, wherein each nucleotide block consists of two or more of the same nucleotide.

7. The method or array of polynucleotides according to any one of claims 1 to 6, wherein at least one identifier sequence of or added to each polynucleotide of the array is a unique molecular identifier sequence (UMI).

8. The method or array of polynucleotides according to claim 7, wherein
(c) the UMI sequence of or added to each polynucleotide is different to the UMI sequence of or added to essentially each other polynucleotide in the array; and/or
(d) the polynucleotide array is divided into a plurality of sub-arrays, wherein the UMI sequence of or added each polynucleotide in each sub-array is different from the UMI sequence of or added to essentially each other polynucleotide of the same sub-array; optionally wherein each polynucleotide further comprises, or the method further comprises adding to each polynucleotide, a barcode sequence (BC), wherein the BC sequence of each polynucleotide is the same as the BC of essentially each other polynucleotide of the same sub-array, but different from the BC sequence of the polynucleotides of essentially every different sub-array, and wherein the BC sequence and the UMI sequence are in or added in either order; further optionally wherein both the UMI sequences and the BC sequences are or are added by a method of any one of claims 1 to 4.

9. The method or array of polynucleotides according to any one of claims 1 to 8, wherein the polynucleotide array is divided into a plurality of sub-arrays, wherein at least one identifier sequence of or added to each polynucleotide is a barcode sequence (BC), wherein the BC sequence of or added to the polynucleotides of each sub-array is the same as the BC sequence of or added to essentially each other polynucleotide of the same sub-array, but different from the BC sequence of or added to the polynucleotides of essentially every different sub-array; optionally wherein each polynucleotide further comprises, or the method further comprises adding to each polynucleotide, a UMI sequence, further optionally wherein the UMI sequence of or added to each polynucleotide in each sub-array is different from the UMI sequence of essentially each other polynucleotide of or added to the same sub-array, and wherein the BC sequence and the UMI sequence are or are added in either order.

10. The method or array of polynucleotides according to any one of claims 1 to 11, wherein each polynucleotide further comprises:
(a) an analyte capture region; and/or
(b) a PCR handle sequence.

11. The method or array of claim 10, wherein the polynucleotides comprise, in a 5′ to 3′ direction:
(a) a PCR handle sequence;
(b) a unique molecular identifier sequence (UMI), and/or a barcode sequence (BC), wherein the UMI is 5′ or 3′ to the BC; and
(c) a 3′ end analyte capture region, optionally a polythymidine.

12. The method or array of claim 10, wherein each polynucleotide comprises, in a 3′ to 5′ direction:
(g) optionally a 3′ hydroxyl group, or a linker that is cleavable to provide a free 3′ hydroxyl group on the polynucleotide after cleavage;
(h) a 3′ end analyte capture region, optionally wherein the 3′ end analyte capture region comprises:
a. a polythymidine sequence;
b. an aptamer;
c. a sequence of at least 10 nucleotides for hybridising to a target polynucleotide analyte;
d. a biotinylated nucleotide sequence; or
e. an ATAC-med sequence.
(i) optionally a first polymerase chain reaction (PCR) handle sequence;
(j) a unique molecular identifier sequence (UMI), and/or a barcode sequence (BC), wherein the UMI is 5′ or 3′ to the BC;
(k) optionally a (second) PCR handle sequence; and
(l) optionally a 5′ end analyte capture region, optionally wherein the 5′ analyte capture region comprises:
a. a polythymidine sequence;
b. an aptamer;
c. a sequence of at least 10 nucleotides for hybridising to a target polynucleotide analyte;
d. a biotinylated nucleotide sequence; or
e. an ATAC-med sequence.

13. The method or array of any one of claims 1 to 10, wherein the identifier sequence is up to 14 nucleotide blocks in length.

14. A micro-particle comprising a micro-bead and an array of polynucleotides according to any one of claims 5 to 13, wherein each polynucleotide is bound to the micro-particle.

15. The micro-particle of claim 14, wherein each polynucleotide of the array has both a BC sequence and an UMI sequence, in either orientation, and wherein the BC sequence of essentially each polynucleotide of the array is the same, and optionally wherein the UMI sequence of essentially each polynucleotide of the array is different.

16. A plurality of micro-particles according to claim 14 or claim 15, wherein each polynucleotide has a BC sequence, wherein the BC sequence of each polynucleotide of each micro-particle is the same as the BC sequence of essentially each other polynucleotide of the same micro-bead, and different from the BC sequence of the polynucleotides of each other micro-particle.

17. A surface comprising a plurality of wells or discrete pre-determined positions, wherein each well or discrete pre-determined position is associated with an array of polynucleotides according to any one of claims 5 to 16.

18. The surface according to claim 17, wherein each polynucleotide has a BC sequence, wherein the BC sequence of each polynucleotide associated with each well or discrete pre-determined position is the same as the BC sequence of essentially each other polynucleotide of the same well or discrete pre-determined position, and different from the BC sequence of the polynucleotides associated with each other each well or discrete pre-determined position of the surface.

19. A kit for generating one or more libraries from one or more groups of analytes, the kit comprising an array of polynucleotides according to any one of claims 5 to 13, a micro-particle according to claim 14 or claim 15, a plurality of micro-particles according to claim 16, or a surface according claim 17 or claim 18.

20. A method of producing a library of polynucleotides, wherein the polynucleotides are amplified from the polynucleotides of a sample and/or tag non-polynucleotide analytes of a sample, and wherein the polynucleotides of the library include a barcode sequence (BC) and/or a unique molecular identifier sequence (UMI), the method comprising
(a) capturing analytes in the sample on an array of polynucleotides synthesised according to any one of claims 1 to 13, an array of polynucleotides according to any one of claims 5 to 13, a micro-particle according to claim 14 or claim 15, a plurality of micro-particles according to claim 16, or a surface according claim 17 or claim 18;
(b) generating copies of the array of polynucleotides, including any sample polynucleotides captured by the array polynucleotides and the BC and/or UMI sequence(s);
(c) amplifying the number of copies of each polynucleotide to produce a library of polynucleotides amplified from or tagging analytes in the sample and including the BC and/or UMI sequence.
(d) The method of claim 20, wherein the sample is a sample of cells, cell nuclei or cellular vesicles, a single cell, a single cell nucleus, a single vesicle, a tissue sample or tissue section, or a biological fluid sample, optionally a blood, blood fraction, serum, plasma, saliva or urine sample.

21. A library of polynucleotides produced by the method of claim 20 or claim 21.

22. A method of determining the accuracy of a method of amplifying and/or sequencing an array of polynucleotides of un-known sequence, the method comprising
(a) including an identifier sequence in each polynucleotide of the array, wherein the identifier sequence comprises at least three nucleotide blocks at known block positions, wherein each nucleotide block of the identifier sequence comprises one of a pre-defined pool of nucleotide block sequences, wherein the pool of nucleotide block sequences at each block position differs from each other nucleotide block sequence in the pool by at least two nucleotide substitutions;
(b) obtaining sequencing data for each polynucleotide or amplified polynucleotide, including the identifier sequence;
(c) determining the percentage of the identifier sequences of the sequenced polynucleotides that are correctly sequenced as consisting only of one of the pre-defined nucleotide block sequences at each nucleotide bock position; and
(d) using the percentage determined in step (c) to determine the accuracy of the method of amplification and/or sequencing and/or of the obtained polynucleotide sequences.

23. The method of claim 22, further comprising using the percentage determined in step (c) to error correct polynucleotides sequences obtained in step (b) that are not correctly sequenced as consisting only of one of the pre-defined nucleotide block sequences at each nucleotide bock position.

24. A method of analysing a library of polynucleotides generated using the method of claim 19 or claim 20 or a library according to claim 21, the method comprising
(c) obtaining sequencing data for each polynucleotide of the library, including the BC and/or UMI;
(d) determining the percentage of the identifier sequences of the sequenced polynucleotides that are correctly sequenced as consisting only of one of the pre-defined nucleotide block sequences at each nucleotide bock position;
(e) using the percentage determined in claim 22(c) to determine a first cut-off for discarding polynucleotide sequences comprising more than the determined first cut-off number of nucleotide blocks in the sequenced identifier sequence that are not correctly sequenced as having one of the pre-selected nucleotide block sequences, and/or to determine a second cut-off for assigning sequenced polynucleotides comprising more than the determined second cut-off number of nucleotide blocks in the sequenced identifier sequence that are not correctly sequenced as having one of the pre-selected nucleotide blocks sequences into different groups instead of the same group; and
(f) collapsing the sequenced polynucleotides, or the remaining sequenced polynucleotides, of the library into groups based on sequence identity across the identifier sequences and using the first and/or second cut-offs determined in step (c).
